(19) United States
(12) Patent Application Publication
(10) Pub. No.: US 2003/0049841 A1
(43) Pub. Date: Mar. 13, 2003

(54) High throughput or capillary-based screening for a bioactivity or biomolecule

(76) Inventors: Jay M. Short, Rancho Santa Fe, CA (US); Martin Keller, San Diego, CA (US); William Michael Lafferty, Encinitas, CA (US)

Correspondence Address: Lisa A. Haile, Ph.D., Gray Cary Ware & Freidenrich LLP, Suite 1100, 4365 Executive Drive, San Diego, CA, 92121-2189, US

(21) Appl. No.: 09/975,036

(22) Filed: Oct. 10, 2001

Related U.S. Application Data
(63) Continuation-in-part of application No. 09/894,956, filed on Jun. 27, 2001.
Application No. 09/894,956 is a continuation-in-part of application No. 09/790,321, filed on Feb. 21, 2001.
Application No. 09/790,321 is a continuation-in-part of application No. 09/687,219, filed on Oct. 12, 2000.
Application No. 09/687,219 is a continuation-in-part of application No. 09/685,432, filed on Oct. 10, 2000.
Application No. 09/685,432 is a continuation-in-part of application No. 09/444,112, filed on Nov. 22, 1999.
Application No. 09/444,112 is a continuation-in-part of application No. 09/098,206, filed on Jun. 16, 1998, now Pat. No. 6,174,673.
Application No. 09/098,206 is a continuation-in-part of application No. 08/876,276, filed on Jun. 16, 1997.
(60) Provisional application No. 60/309,101, filed on Jul. 31, 2001.

(51) Int. Cl. C12N 15/02

(52) U.S. Cl. 435/449

(57) ABSTRACT

Provided is a method of screening or enriching a sample containing polynucleotides from a mixed population of organisms. The method includes creating a DNA library from a plurality of nucleic acid sequences of a mixed population of organisms and separating clones containing a polynucleotide sequence of interest on an analyzer detects a detectable molecule on a probe or bioactive substrate. The analyzer includes FACS devices, SQUID devices and MCS devices. The separated or enrich library can then be further process by activity based screening or sequence based screening. In addition, the enriched sequence can be compared to a database and to identify sequences in the database which have homology to a clone in the library thereby obtaining a nucleic acid profile of the mixed population of organisms.

### CROSS REFERENCE TO RELATED APPLICATIONS

This application claims priority to U.S. patent application Ser. No. 09/894,956, filed Jun. 27, 2001, which is a continuation-in-part of U.S. patent application Ser. No. 09/790,321, filed Feb. 21, 2001, which is a continuation-in-part of U.S. patent application Ser. No. 09/687,219, filed Oct. 12, 2000, which is a continuation-in-part of U.S. patent application Ser. No. 09/685,432, filed Oct. 10, 2000; which is a continuation-in-part of U.S. patent application Ser. No. 09/444,112, filed Nov. 22, 1999; which is a continuation-in-part of U.S. patent application Ser. No. 09/098,206, filed Jun. 16, 1998, now U.S. Pat. No. 6,174,673, which is a continuation-in-part of U.S. patent application Ser. No. 08/876,276, filed Jun. 16, 1997; this application also claims priority to U.S. patent application Ser. No. 09/738,871, filed Dec. 14, 2000, which is a continuation-in-part of U.S. patent application Ser. No. 09/685,432, filed Oct. 10, 2000, which is a continuation in part of U.S. patent application Ser. No. 09/444,112, filed Nov. 22, 1999; which is a continuation-in-part of U.S. patent application Ser. No. 09/098,206, filed Jun. 16, 1998, now U.S. Pat. No. 6,174,673, which is a continuation-in-part of U.S. patent application Ser. No. 08/876,276, filed Jun. 16, 1997; this application also claims priority to U.S. Provisional Application 60/309,101, the contents of which are all incorporated by reference in their entirety herein.

### FIELD OF THE INVENTION

The present invention relates generally to screening of mixed populations of organisms or nucleic acids and more specifically to the identification of bioactive molecules and bioactivities using screening techniques, including high throughput screening and capillary array platform for screening samples.

### BACKGROUND

There is a critical need in the chemical industry for efficient catalysts for the practical synthesis of optically pure materials; enzymes can provide the optimal solution. All classes of molecules and compounds that are utilized in both established and emerging chemical, pharmaceutical, textile, food and feed, detergent markets must meet stringent economical and environmental standards. The synthesis of polymers, pharmaceuticals, natural products and agrochemicals is often hampered by expensive processes which produce harmful byproducts and which suffer from low enantioselectivity (Faber, 1995; Tonkovich and Gerber, U.S. Dept of Energy study, 1995). Enzymes have a number of remarkable advantages which can overcome these problems in catalysis: they act on single functional groups, they distinguish between similar functional groups on a single molecule, and they distinguish between enantiomers. Moreover, they are biodegradable and function at very low mole fractions in reaction mixtures. Because of their chemo-, regio- and stereospecificity, enzymes present a unique opportunity to optimally achieve desired selective transformations. These are often extremely difficult to duplicate chemically, especially in single-step reactions. The elimination of the need for protection groups, selectivity, the ability to carry out multi-step transformations in a single reaction vessel, along with the concomitant reduction in environmental burden, has led to the increased demand for enzymes in chemical and pharmaceutical industries (Faber, 1995). Enzyme-based processes have been gradually replacing many conventional chemical-based methods (Wrotnowski, 1997). A current limitation to more widespread industrial use is primarily due to the relatively small number of commercially available enzymes. Only ˜300 enzymes (excluding DNA modifying enzymes) are at present commercially available from the >3000 non DNA-modifying enzyme activities thus far described.

The use of enzymes for technological applications also may require performance under demanding industrial conditions. This includes activities in environments or on substrates for which the currently known arsenal of enzymes was not evolutionarily selected. Enzymes have evolved by selective pressure to perform very specific biological functions within the milieu of a living organism, under conditions of mild temperature, pH and salt concentration. For the most part, the non-DNA modifying enzyme activities thus far described (Enzyme Nomenclature, 1992) have been isolated from mesophilic organisms, which represent a very small fraction of the available phylogenetic diversity (Amann et al., 1995). The dynamic field of biocatalysis takes on a new dimension with the help of enzymes isolated from microorganisms that thrive in extreme environments. Such enzymes must function at temperatures above 100° C. in terrestrial hot springs and deep sea thermal vents, at temperatures below 0° C. in arctic waters, in the saturated salt environment of the Dead Sea, at pH values around 0 in coal deposits and geothermal sulfur-rich springs, or at pH values greater than 11 in sewage sludge (Adams and Kelly, 1995). The enzymes may also be obtained from: geothermal and hydrothermal fields, acidic soils, sulfotara and boiling mud pots, pools, hot-springs and geysers where the enzymes are neutral to alkaline, marine actinomycetes, metazoan, endo and ectosymbionts, tropical soil, temperate soil, arid soil, compost piles, manure piles, marine sediments, freshwater sediments, water concentrates, hypersaline and super-cooled sea ice, arctic tundra, Sargosso sea, open ocean pelagic, marine snow, microbial mats (such as whale falls, springs and hydrothermal vents), insect and nematode gut microbial communities, plant endophytes, epiphytic water samples, industrial sites and ex situ enrichments. Additionally, the enzymes may be isolated from eukaryotes, prokaryotes, myxobacteria (epothilone), air, water, sediment, soil or rock. Enzymes obtained from these extremophilic organisms open a new field in biocatalysis.

For example, several esterases and lipases cloned and expressed from extremophilic organisms are remarkably robust, showing high activity throughout a wide range of temperatures and pHs. The fingerprints of several of these esterases show a diverse substrate spectrum, in addition to differences in the optimum reaction temperature. Certain esterases recognize only short chain substrates while others only acts on long chain substrates in addition to a huge difference in the optimal reaction temperature. These results suggest that more diverse enzymes fulfilling the need for new biocatalysts can be found by screening biodiversity. Substrates upon which enzymes act are herein defined as bioactive substrates.

Furthermore, virtually all of the enzymes known so far have come from cultured organisms, mostly bacteria and more recently archaea (Enzyme Nomenclature, 1992). Traditional enzyme discovery programs rely solely on cultured microorganisms for their screening programs and are thus only accessing a small fraction of natural diversity. Several recent studies have estimated that only a small percentage, conservatively less than 1%, of organisms present in the natural environment have been cultured (see Table I, Amann et al., 1995, Barns et. al 1994, Torvsik, 1990). For example, Norman Pace's laboratory recently reported intensive untapped diversity in water and sediment samples from the “Obsidian Pool” in Yellowstone National Park, a spring which has been studied since the early 1960's by microbiologists (Barns, 1994). Amplification and cloning of 16S rRNA encoding sequences revealed mostly unique sequences with little or no representation of the organisms which had previously been cultured from this pool. This suggests substantial diversity of archaea with so far unknown morphological, physiological and biochemical features which may be useful in industrial processes. David Ward's laboratory in Bozmen, Mont. has performed similar studies on the cyanobacterial mat of Octopus Spring in Yellowstone Park and came to the same conclusion, namely, tremendous uncultured diversity exists (Bateson et al., 1989). Giovannoni et al. (1990) reported similar results using bacterioplankton collected in the Sargasso Sea while Torsvik et al. (1990) have shown by DNA reassociation kinetics that there is considerable diversity in soil samples. Hence, this vast majority of microorganisms represents an untapped resource for the discovery of novel biocatalysts. In order to access this potential catalytic diversity, recombinant screening approaches are required.

The discovery of novel bioactive molecules other than enzymes is also afforded by the present invention. For instance, antibiotics, antivirals, antitumor agents and regulatory proteins can be discovered utilizing the present invention.

Bacteria and many eukaryotes have a coordinated mechanism for regulating genes whose products are involved in related processes. The genes are clustered, in structures referred to as “gene clusters,” on a single chromosome and are transcribed together under the control of a single regulatory sequence, including a single promoter which initiates transcription of the entire cluster. The gene cluster, the promoter, and additional sequences that function in regulation altogether are referred to as an “operon” and can include up to 30 or more genes, usually from 2 to 6 genes. Thus, a gene cluster is a group of adjacent genes that are either identical or related, usually as to their function.

Some gene families consist of one or more identical members. Clustering is a prerequisite for maintaining identity between genes, although clustered genes are not necessarily identical. Gene clusters range from extremes where a duplication is generated of adjacent related genes to cases where hundreds of identical genes lie in a tandem array. Sometimes no significance is discernable in a repetition of a particular gene. A principal example of this is the expressed duplicate insulin genes in some species, whereas a single insulin gene is adequate in other mammalian species.

It is important to further research gene clusters and the extent to which the full length of the cluster is necessary for the expression of the proteins resulting therefrom. Gene clusters undergo continual reorganization and, thus, the ability to create heterogeneous libraries of gene clusters from, for example, bacterial or other prokaryote sources is valuable in determining sources of novel proteins, particularly including enzymes such as, for example, the polyketide synthases that are responsible for the synthesis of polyketides having a vast array of useful activities. As indicated, other types of proteins and molecules that are the product(s) of gene clusters are also contemplated, including, for example, antibiotics, antivirals, antitumor agents and regulatory proteins, such as insulin.

Polyketides are molecules which are an extremely rich source of bioactivities, including antibiotics (such as tetracyclines and erythromycin), anti-cancer agents (daunomycin), immunosuppressants (FK506 and rapamycin), and veterinary products (monensin). Many polyketides (produced by polyketide synthases) are valuable as therapeutic agents. Polyketide synthases are multifunctional enzymes that catalyze the biosynthesis of a huge variety of carbon chains differing in length and patterns of functionality and cyclization. Polyketide synthase genes fall into gene clusters and at least one type (designated type I) of polyketide synthases have large size genes and encoded enzymes, complicating genetic manipulation and in vitro studies of these genes/proteins. The method(s) of the present invention facilitate the rapid discovery of these gene clusters in gene expression libraries.

Gene libraries of microorganisms have been prepared for the purpose of identifying genes involved in biosynthetic pathways that produce medicinally-active metabolites and specialty chemicals. These pathways require multiple proteins (specifically, enzymes), entailing greater complexity than the single proteins used as drug targets. For example, genes encoding pathways of bacterial polyketide synthases (PKSs) were identified by screening gene libraries of the organism (Malpartida et al. 1984, Nature 309:462; Donadio et al. 1991, Science 252:675-679). PKSs catalyze multiple steps of the biosynthesis of polyketides, an important class of therapeutic compounds, and control the structural diversity of the polyketides produced. A host-vector system in Streptomyces has been developed that allows directed mutation and expression of cloned PKS genes (McDaniel et al. 1993, Science 262:1546-1550; Kao et al. 1994, Science 265:509-512). This specific host-vector system has been used to develop more efficient ways of producing polyketides, and to rationally develop novel polyketides (Khosla et al., WO 95/08548).

Another example is the production of the textile dye, indigo, by fermentation in an E. coli host. Two operons containing the genes that encode the multienzyme biosynthetic pathway have been genetically manipulated to improve production of indigo by the foreign E. coli host. (Ensley et al. 1983, Science 222:167-169; Murdock et al. 1993, Bio/Technology 11:381-386). Overall, conventional studies of heterologous expression of genes encoding a metabolic pathway involve directed cloning, sequence analysis, designed mutations, and rearrangement of specific genes that encode proteins known to be involved in previously characterized metabolic pathways.

In view of numerous advances in the understanding of disease mechanisms and identification of drug targets, there is an increasing need for innovative strategies and methods for rapidly identifying lead compounds and channeling them toward clinical testing. The methods of the present invention facilitate the rapid discovery of genes, gene pathways and gene clusters, particularly polyketide synthase genes, polyketide synthase gene pathways and polyketides, from gene expression libraries.

Of particular interest are cellular “switches” known as receptors which interact with a variety of biomolecules, such as hormones, growth factors, and neurotransmitters, to mediate the transduction of an “external” cellular signaling event into an “internal” cellular signal. External signaling events include the binding of a ligand to the receptor, and internal events include the modulation of a pathway in the cytoplasm or nucleus involved in the growth, metabolism or apoptosis of the cell. Internal events also include the inhibition or activation of transcription of certain nucleic acid sequences, resulting in the increase or decrease in the production or presence of certain molecules (such as nucleic acid, proteins, and/or other molecules affected by this increase or decrease in transcription). Drugs to cure disease or alleviate its symptoms can activate or block any of these events to achieve a desired pharmaceutical effect.

Transduction can be accomplished by a transducing protein in the cell membrane which is activated upon an allosteric change the receptor may undergo upon binding to a specific biomolecule. The “active” transducing protein activates production of so-called “second messenger” molecules within the cell, which then activate certain regulatory proteins within the cell that regulate gene expression or alter some metabolic process. Variations on the theme of this “cascade” of events occur. For example, a receptor may act as its own transducing protein, or a transducing protein may act directly on an intracellular target without mediation by a second messenger.

Signal transduction is a fundamental area of inquiry in biology. For instance, ligand/receptor interactions and the receptor/effector coupling mediated by Guanine nucleotide-binding proteins (G-proteins) are of interest in the study of disease. A large number of G protein-linked receptors funnel extracellular signals as diverse as hormones, growth factors, neurotransmitters, primary sensory stimuli, and other signals through a set of G proteins to a small number of second-messenger systems. The G proteins act as molecular switches with an “on” and “off” state governed by a GTPase cycle. Mutations in G proteins may result in either constitutive activation or loss of expression mutations.

Many receptors convey messages through heterotrimeric G proteins, of which at least 17 distinct forms have been isolated. Additionally, there are several different G protein-dependent effectors. The signals transduced through the heterotrimeric G proteins in mammalian cells influence intracellular events through the action of effector molecules.

Given the variety of functions subserved by G protein-coupled signal transduction, it is not surprising that abnormalities in G protein-coupled pathways can lead to diseases with manifestations as dissimilar as blindness, hormone resistance, precocious puberty and neoplasia. G-protein-coupled receptors are extremely important to drug research efforts. It is estimated that up to 60% of today's prescription drugs work by somehow interacting with G protein-coupled receptors. However, these drugs were developed using classical medicinal chemistry and without a knowledge of the molecular mechanism of action. A more efficient drug discovery program could be deployed by targeting individual receptors and making use of information on gene sequence and biological function to develop effective therapeutics. The present invention allows one to, for example, study molecules which affect the interaction of G proteins with receptors, or of ligands with receptors.

Several groups have reported cells which express mammalian G proteins or subunits thereof, along with mammalian receptors which interact with these molecules. For example, WO92/05244 (Apr. 2, 1992) describes a transformed yeast cell which is incapable of producing a yeast G protein □ subunit, but which has been engineered to produce both a mammalian G protein □ subunit and a mammalian receptor which interacts with the subunit. The authors found that a modified version of a specific mammalian receptor integrated into the membrane of the cell, as shown by studies of the ability of isolated membranes to interact properly with various known agonists and antagonists of the receptor. Ligand binding resulted in G protein-mediated signal transduction.

Another group has described the functional expression of a mammalian adenylyl cyclase in yeast, and the use of the engineered yeast cells in identifying potential inhibitors or activators of the mammalian adenylyl cyclase (WO 95/30012). Adenylyl cyclase is among the best studied of the effector molecules which function in mammalian cells in response to activated G proteins. “Activators” of adenylyl cyclase cause the enzyme to become more active, elevating the cAMP signal of the yeast cell to a detectable degree. “Inhibitors” cause the cyclase to become less active, reducing the cAMP signal to a detectable degree. The method describes the use of the engineered yeast cells to screen for drugs which activate or inhibit adenylyl cyclase by their action on G protein-coupled receptors.

When attempting to identify genes encoding bioactivities of interest from complex mixed population nucleic acid libraries, the rate limiting steps in discovery occur at the both DNA cloning level and at the screening level. Screening of complex mixed population libraries which contain, for example, 100s of different organisms requires the analysis of several million clones to cover this genomic diversity. An extremely high-throughput screening method has been developed to handle the enormous numbers of clones present in these libraries.

In traditional flow cytometry, it is common to analyze very large numbers of eukaryotic cells in a short period of time. Newly developed flow cytometers can analyze and sort up to 20,000 cells per second. In a typical flow cytometer, individual particles pass through an illumination zone and appropriate detectors, gated electronically, measure the magnitude of a pulse representing the extent of light scattered. The magnitude of these pulses are sorted electronically into “bins” or “channels”, permitting the display of histograms of the number of cells possessing a certain quantitative property versus the channel number (Davey and Kell, 1996). It was recognized early on that the data accruing from flow cytometric measurements could be analyzed (electronically) rapidly enough that electronic cell-sorting procedures could be used to sort cells with desired properties into separate “buckets”, a procedure usually known as fluorescence-activated cell sorting (Davey and Kell, 1996).

Fluorescence-activated cell sorting has been primarily used in studies of human and animal cell lines and the control of cell culture processes. Fluorophore labeling of cells and measurement of the fluorescence can give quantitative data about specific target molecules or subcellular components and their distribution in the cell population. Flow cytometry can quantitate virtually any cell-associated property or cell organelle for which there is a fluorescent probe (or natural fluorescence). The parameters which can be measured have previously been of particular interest in animal cell culture.

Flow cytometry has also been used in cloning and selection of variants from existing cell clones. This selection, however, has required stains that diffuse through cells passively, rapidly and irreversibly, with no toxic effects or other influences on metabolic or physiological processes. Since, typically, flow sorting has been used to study animal cell culture performance, physiological state of cells, and the cell cycle, one goal of cell sorting has been to keep the cells viable during and after sorting.

There currently are no reports in the literature of screening and discovery of recombinant enzymes in E. coli expression libraries by fluorescence activated cell sorting of single cells. Furthermore there are no reports of recovering DNA encoding bioactivities screened by expression screening in E. coli using a FACS machine. The present invention provides these methods to allow the extremely rapid screening of viable or non-viable cells to recover desirable activities and the nucleic acid encoding those activities.

A limited number of papers describing various applications of flow cytometry in the field of microbiology and sorting of fluorescence activated microorganisms have, however, been published (Davey and Kell, 1996). Fluorescence and other forms of staining have been employed for microbial discrimination and identification, and in the analysis of the interaction of drugs and antibiotics with microbial cells. Flow cytometry has been used in aquatic biology, where autofluorescence of photosynthetic pigments are used in the identification of algae or DNA stains are used to quantify and count marine populations (Davey and Kell, 1996). Thus, Diaper and Edwards used flow cytometry to detect viable bacteria after staining with a range of fluorogenic esters including fluorescein diacetate (FDA) derivatives and CemChrome B, a proprietary stain sold commercially for the detection of viable bacteria in suspension (Diaper and Edwards, 1994). Labeled antibodies and oligonucleotide probes have also been used for these purposes.

Papers have also been published describing the application of flow cytometry to the detection of native and recombinant enzymatic activities in eukaryotes. Betz et al. studied native (non-recombinant) lipase production by the eukaryote, Rhizopus arrhizus with flow cytometry. They found that spore suspensions of the mold were heterogeneous as judged by light-scattering data obtained with excitation at 633 nm, and they sorted clones of the subpopulations into the wells of microtiter plates. After germination and growth, lipase production was automatically assayed (turbidimetrically) in the microtiter plates, and a representative set of the most active were reisolated, cultured, and assayed conventionally (Betz et al., 1984).

Scrienc et al. have reported a flow cytometric method for detecting cloned-galactosidase activity in the eukaryotic organism, S. cerevisiae. The ability of flow cytometry to make measurements on single cells means that individual cells with high levels of expression (e.g., due to gene amplification or higher plasmid copy number) could be detected. In the method reported, a non-fluorescent compound β-naphthol-β-galactopyranoside) is cleaved by β-galactosidase and the liberated naphthol is trapped to form an insoluble fluorescent product. The insolubility of the fluorescent product is of great importance here to prevent its diffusion from the cell. Such diffusion would not only lead to an underestimation of β-galactosidase activity in highly active cells but could also lead to an overestimation of enzyme activity in inactive cells or those with low activity, as they may take up the leaked fluorescent compound, thus reducing the apparent heterogeneity of the population.

One group has described the use of a FACS machine in an assay detecting fusion proteins expressed from a specialized transducing bacteriophage in the prokaryote Bacillus subtilis (Chung, et.al., J. of Bacteriology, Apr. 1994, p. 1977-1984; Chung, et.al., Biotechnology and Bioengineering, Vol. 47, pp. 234-242 (1995)). This group monitored the expression of a lacZ gene (encodes b-galactosidase) fused to the sporulation loci in subtilis (spo). The technique used to monitor b-galactosidase expression from spo-lacZ fusions in single cells involved taking samples from a sporulating culture, staining them with a commercially available fluorogenic substrate for b-galactosidase called C8-FDG, and quantitatively analyzing fluorescence in single cells by flow cytometry. In this study, the flow cytometer was used as a detector to screen for the presence of the spo gene during the development of the cells. The device was not used to screen and recover positive cells from a gene expression library or nucleic acid for the purpose of discovery.

Another group has utilized flow cytometry to distinguish between the developmental stages of the delta-proteobacteria Myxococcus xanthus (F. Russo-Marie, et.al., PNAS, Vol. 90, pp.8194-8198, September 1993). As in the previously described study, this study employed the capabilities of the FACS machine to detect and distinguish genotypically identical cells in different development regulatory states. The screening of an enzymatic activity was used in this study as an indirect measure of developmental changes.

The lacZ gene from E. coli is often used as a reporter gene in studies of gene expression regulation, such as those to determine promoter efficiency, the effects of trans-acting factors, and the effects of other regulatory elements in bacterial, yeast, and animal cells. Using a chromogenic substrate, such as ONPG (o-nitrophenyl-(-D-galactopyranoside), one can measure expression of -galactosidase in cell cultures; but it is not possible to monitor expression in individual cells and to analyze the heterogeneity of expression in cell populations. The use of fluorogenic substrates, however, makes it possible to determine β-galactosidase activity in a large number of individual cells by means of flow cytometry. This type of determination can be more informative with regard to the physiology of the cells, since gene expression can be correlated with the stage in the mitotic cycle or the viability under certain conditions. In 1994, Plovins et al., reported the use of fluorescein-Di-β-D-galactopyranoside (FDG) and C12-FDG as substrates for β-galactosidase detection in animal, bacterial, and yeast cells. This study compared the two molecules as substrates for β-galactosidase, and concluded that FDG is a better substrate for β-galactosidase detection by flow cytometry in bacterial cells. The screening performed in this study was for the comparison of the two substrates. The detection capabilities of a FACS machine were employed to perform the study on viable bacterial cells.

Cells with chromogenic or fluorogenic substrates yield colored and fluorescent products, respectively. Previously, it had been thought that the flow cytometry-fluorescence activated cell sorter approaches could be of benefit only for the analysis of cells that contain intracellularly, or are normally physically associated with, the enzymatic activity of small molecule of interest. On this basis, one could only use fluorogenic reagents which could penetrate the cell and which are thus potentially cytotoxic. To avoid clumping of heterogeneous cells, it is desirable in flow cytometry to analyze only individual cells, and this could limit the sensitivity and therefore the concentration of target molecules that can be sensed. Weaver and his colleagues at MIT and others have developed the use of gel microdroplets containing (physically) single cells which can take up nutrients, secret products, and grow to form colonies. The diffusional properties of gel microdroplets may be made such that sufficient extracellular product remains associated with each individual gel microdroplet, so as to permit flow cytometric analysis and cell sorting on the basis of concentration of secreted molecule within each microdroplet. Beads have also been used to isolate mutants growing at different rates, and to analyze antibody secretion by hybridoma cells and the nutrient sensitivity of hybridoma cells. The gel microdroplet method has also been applied to the rapid analysis of mycobacterial growth and its inhibition by antibiotics.

The gel microdroplet technology has had significance in amplifying the signals available in flow cytometric analysis, and in permitting the screening of microbial strains in strain improvement programs for biotechnology. Wittrup et al., (Biotechnolo.Bioeng. (1993) 42:351-356) developed a microencapsulation selection method which allows the rapid and quantitative screening of >106 yeast cells for enhanced secretion of Aspergillus awamori glucoamylase. The method provides a 400-fold single-pass enrichment for high-secretion mutants.

Gel microdroplet or other related technologies can be used in the present invention to localize as well as amplify signals in the high throughput screening of recombinant libraries. Cell viability during the screening is not an issue or concern since nucleic acid can be recovered from the microdroplet.

Different types of encapsulation strategies and compounds or polymers can be used with the present invention. For instance, high temperature agaroses can be employed for making microdroplets stable at high temperatures, allowing stable encapsulation of cells subsequent to heat kill steps utilized to remove all background activities when screening for thermostable bioactivities.

There are several hurdles which must be overcome when attempting to detect and sort E. coli expressing recombinant enzymes, and recover encoding nucleic acids. FACS systems have typically been based on eukaryotic separations and have not been refined to accurately sort single E. coli cells; the low forward and sideward scatter of small particles like E. coli, reduces the ability of accurate sorting; enzyme substrates typically used in automated screening approaches, such as umbelifferyl based substrates, diffuse out of E. coli at rates which interfere with quantitation. Further, recovery of very small amounts of DNA from sorted organisms can be problematic. The methods of the present invention address and overcome these hurdles with the novel screening approaches described herein.

There has been a dramatic increase in the need for bioactive compounds with novel activities. This demand has arisen largely from changes in worldwide demographics coupled with the clear and increasing trend in the number of pathogenic organisms that are resistant to currently available antibiotics as well as the need for new industrial processes for synthesis of compounds. For example, while there has been a surge in demand for antibacterial drugs in emerging nations with young populations, countries with aging populations, such as the U.S., require a growing repertoire of drugs against cancer, diabetes, arthritis and other debilitating conditions. The death rate from infectious diseases has increased 58% between 1980 and 1992 and it has been estimated that the emergence of antibiotic resistant microbes has added in excess of $30 billion annually to the cost of health care in the U.S. alone. (Adams et al., Chemical and Engineering News, 1995; Amann et al., Microbiological Reviews, 59, 1995). As a response to this trend, pharmaceutical companies have significantly increased their screening of microbial diversity for compounds with unique activities or specificities.

The majority of bioactive compounds currently in use are derived from soil microorganisms. Many microbes inhabiting soils and other complex ecological communities produce a variety of compounds that increase their ability to survive and proliferate. These compounds are generally thought to be nonessential for growth of the organism and are synthesized with the aid of genes involved in intermediary metabolism. Such secondary metabolites that influence the growth or survival of other organisms are known as “bioactive” compounds and serve as key components of the chemical defense arsenal of both micro- and macroorganisms. Humans have exploited these compounds for use as antibiotics, antiinfectives and other bioactive compounds with activity against a broad range of prokaryotic and eukaryotic pathogens (Barnes et al., Proc.Nat. Acad. Sci. U.S.A., 91, 1994).

The approach currently used to screen microbes for new bioactive compounds has been largely unchanged since the inception of the field. New isolates of bacteria, particularly gram positive strains from soil environments, are collected and their metabolites tested for pharmacological activity.

There is still tremendous biodiversity that remains untapped as the source of lead compounds. However, the currently available methods for screening and producing lead compounds cannot be applied efficiently to these under-explored resources. For instance, it is estimated that at least 99% of marine bacteria species do not survive on laboratory media, and commercially available fermentation equipment is not optimal for use in the conditions under which these species will grow, hence these organisms are difficult or impossible to culture for screening or re-supply. Recollection, growth, strain improvement, media improvement and scale-up production of the drug-producing organisms often pose problems for synthesis and development of lead compounds. Furthermore, the need for the interaction of specific organisms to synthesize some compounds makes their use in discovery extremely difficult. New methods to harness the genetic resources and chemical diversity of these untapped sources of compounds for use in drug discovery are very valuable.

A central core of modern biology is that genetic information resides in a nucleic acid genome, and that the information embodied in such a genome (i.e., the genotype) directs cell function. This occurs through the expression of various genes in the genome of an organism and regulation of the expression of such genes. The expression of genes in a cell or organism defines the cell or organism's physical characteristics (i.e., its phenotype). This is accomplished through the translation of genes into proteins. Determining the biological activity of a protein obtained from an environmental sample can provide valuable information about the role of proteins in the environments. In addition, such information can help in the development of biologics, diagnostics, therapeutics, and compositions for industrial applications.

Accordingly, the present invention provides methods and compositions to access this untapped biodiversity and to rapidly screen for polynucleotides, proteins and small molecules of interest utilizing high throughput screening of multiple samples. These biomolecules can be derived from cultured or uncultured samples of organisms. In one embodiment, the methods of the present invention provides a method for high throughput cultivation of unculturable microorganisms.

In the United States, cancer is the second leading cause of disease-related deaths, second only to cardiovascular disease and it is projected to become the leading cause of death within a few years. The most common curative therapies for cancers found at an early stage include surgery and radiation (1). These methods are not nearly as successful in the more advanced stages of cancer. Current chemotherapeutic agents have been useful but are limited in their effectiveness. Significant results are obtained with chemotherapy in a small range of cancers including childhood cancers and certain adult malignancies such as lymphoma and leukemia (2). Despite these positive results, most chemotherapeutic treatments are not curative and serve primarily as palliatives (1). Thus, it is clear that current medical science still has a long way to go before providing long-term survival to patients and curability of most cancers. However, basic research over the past 20 years has provided a vast amount of scientific information defining key players in the progression of cancers. Understanding the disease processes at the molecular level provides the means to determine optimal molecular targets and presumably selectively kill cancerous tissues. Some of the key areas that have been identified in the progression of tumors include proliferative signal transduction, aberrant cell-cycle regulation, apoptosis, telomere biology, genetic instability and angiogenesis (3). This basic research is now beginning to pay off as progress towards more effective treatments is beginning to emerge (4,5). New chemotherapeutic agents directed against these identified areas are in Phase I-III clinical trials with some of the most promising agents active against tyrosine kinases involved in signal transduction. Small molecule inhibitors of Bcr-abl, protein kinase C, VEGF receptors, and EGF receptors, to name a few, are all in clinical trials (4). Some specific examples include the EGF receptor inhibitors, ZD1839 and CP358774, which are in Phase II trials and appear to be well tolerated by patients with positive signs of clinical activity (6). Even with this progress, the complexities of tumorigenesis necessitate not only the ongoing discovery and development of novel therapeutic agents but also the basic research to elucidate the underlying mechanisms of the disease. Presently, there are at least 50 known cancer related targets and it has been speculated that there may be up to several hundred new targets discovered (2). To make use of this influx of information, novel methods for the ultra high throughput screening of potential anti-cancer drugs must be developed. Recent technological developments in molecular biology, automation, miniaturization, and information technology have facilitated the high throughput screening of novel compounds from a variety of sources. However, despite the increased throughput, there is some disappointment in the industry regarding the number of novel drugs that have resulted from these efforts (7). One of the significant challenges is to find sufficient numbers of compounds with the structural diversity necessary to increase the chances of finding activity at the molecular target. Currently, screened compounds come from chemical and combinatorial libraries, historical compound collections and natural product libraries (8). Of these, one of the richest sources of drugs has been from natural product libraries. Cragg et al (9) reported that over 60% of the approved anticancer drugs and pre-NDA candidates between 1984 and 1995 were from natural sources or derived from natural products. In fact, it is estimated that 39% of all 520 new approved drugs during this time period were from or derived from natural products with 80% of anti-infectives coming from nature. Typically, natural products are small molecules that have a much greater structural diversity than most combinatorial approaches. Small molecules in general are favored by the pharmaceutical industry because they are more “drug-like” in nature with the ability to penetrate tumors, be absorbed, and metabolized easily. However, natural products have their disadvantages, largely due to the reproducibility of the source, the labor-intensive extraction process, the abundance of the supply, and the concerns over rights to biodiversity (8).

The therapeutic agents from natural sources have been primarily of plant and microbial origins. Of these, the greatest biodiversity exists in the microorganisms that populate virtually every corner of the earth. The approach currently used to screen microbes for new bioactive compounds has changed little over the last 50 years. Microbiologists collect samples from the environment, isolate a pure culture, grow up sufficient material, extract the culture, and test their metabolites for pharmacological activity. Variations of these natural products can then be generated through mutagenesis of the producing organism or through chemical or biochemical modification of the original backbone molecules. Natural products are typically made by multi-enzyme systems in which each enzyme carries out one of the many transformations required to make the final small molecule products, an example being antibiotics. These bioactive molecules are derived from the organism's ability to produce secondary metabolites in response to the specific needs and challenges of their local environments. The genes encoding these enzymes are often clustered into so-called “biosynthetic operons” which contain the blueprint for building a natural product (10). This blueprint for production of a small bioactive molecule is typically more than 25,000 nucleotides and can be greater than 100,000 nucleotides. There are many examples of entire pathways encoding for the production of such small molecules as oxytetracycline, jadomycin, daunorubicin, to name just a few, that have been cloned as contiguous pieces of DNA from a producing organism (11). Some of these pathways (e.g. actinorhodin, tetracenomycin, puromycin, nikkomycin) have been transferred to other microbial hosts and the small molecule heterologously expressed (11).

A more recent approach has been to use recombinant techniques to synthesize hybrid antibiotic pathways by combining gene subunits from previously characterized pathways. This approach, called “combinatorial biosynthesis” has been focused primarily on the polyketide antibiotics and has resulted in a number of compounds which have displayed activity (12,13). In one such approach using the erythronolide biosynthetic operon, enzymatic domains have been added to (14) and repositioned within the operon (15), thereby reprogramming polyketide biosynthesis. However, compounds with novel antibiotic activities have not yet been reported: an observation that maybe be due to the fact that the pathway subunits are derived from those encoding previously characterized compounds. What has not been accounted for in previous attempts to discover novel bioactive compounds is the relatively recent observation that only a small fraction of microbes in natural environments can be grown under laboratory conditions. Estimates are that far less than 1% of all prokaryotes are capable of being grown in pure culture in the laboratory. This implies a need for culture-independent methods for bioactive compound discovery.

Culture-independent approaches to directly clone genes encoding both target enzymes and other bioactive molecules from environmental samples are based on the construction of libraries which represent the collective genomes of naturally occurring organisms, archived in cloning vectors that can be propagated in E. coli, Streptomyces, or other suitable hosts. Because the cloned DNA is initially extracted directly from environmental samples containing a mixed population of organisms, the representation of the libraries is not limited to the small fraction of prokaryotes that can be grown in pure culture, nor is it biased towards a few rapidly growing species. Samples can be obtained from virtually all ecosystems represented on earth, including such extreme environments as geothermal and hydrothermal vents, acidic soils and boiling mud pots, contaminated industrial sites, marine symbionts, etc.

Screening of complex mixed population libraries containing, for example, 100 different organisms requires the analysis of tens of millions of clones to cover the genomic diversity. An extremely high throughput screening method must be implemented to handle the enormous numbers of clones present in these libraries. In the pharmaceutical industry today, high throughput screening typically has throughput rates on the order of 10,000 compounds per assay per day with some laboratories working at 100,000 assays per day. Most of the development in the industry has centered around the miniaturization and automation of these screens to higher density, smaller volume plate formats. However, this strategy could be reaching the practical limits of conventional liquid-dispensing technology and current microplate fabrication processes, as well as the limits in controlling evaporation in open systems with very small well volumes.

Current platforms for screening micro-scale particles of interest include plates that are formed with small wells, or through-holes. The wells or through-holes are used to hold a sample to be analyzed. The sample typically contains the particles of interest. When wells are used, complex and inefficient sample delivery and extraction systems must be used in order to deposit the sample into the wells on the plate, and remove the sample from the wells for further analysis. Wells-based platforms have a bottom, for which gravity is primarily used for suspending the sample on the plate to develop the particulate or incubate cells of interest.

Another type of platform uses through-holes, which are typically machined into a plate by one of a number of well-known methods. Through-holes rely on capillary forces for introducing the sample to the plate, and utilize surface tension for suspending the sample in the through-holes. However, typical through-hole-based devices are limited to relatively small aspect ratios, or the ratio of length to internal diameter of the hole. A small aspect ratio yields greater evaporative loss of a liquid contained in the hole, and such evaporation is difficult to control. Through-holes are also limited in their functionality. For example, the process of forming through-holes in a plate usually does not allow for the use of various materials to line the inside of the holes, or to clad the outside of the holes.

### SUMMARY OF THE INVENTION

The present invention comprises methods for high throughput screening for biomolecules of interest. In the present invention, nucleic acids or nucleic acid libraries derived from mixed populations of nucleic acids and/or organisms are screened very rapidly for bioactivities of interest utilizing liquid phase screening methods. These libraries can represent the genomes of multiple organisms, species or subspecies. In one aspect, the libraries are screened via hybridization methods, such as “biopanning”, or by activity based screening methods. High throughput screening can be performed by utilizing single cell screening systems, such as fluorescence activated cell sorting (FACS) or by capillary array-based systems.

Accordingly, in one embodiment, the present invention provides a process for identifying clones having a specified activity of interest, which process comprises (i) generating one or more gene libraries derived from nucleic acid isolated from a mixed population of organisms; and (ii) screening said libraries utilizing a high throughput cell analyzer, e.g., a fluorescence activated cell sorter or a non-optical cell sorter, to identify said clones.

More particularly, the invention provides a process for identifying clones having a specified activity of interest by (i) generating one or more libraries, e.g., expression libraries, made to contain nucleic acid directly or indirectly isolated from a mixed population of organisms; (ii) exposing said libraries to a particular substrate or substrates of interest; and (iii) screening said exposed libraries utilizing a high throughput cell analyzer, e.g., a fluorescence activated cell sorter or a non-optical cell sorter, to identify clones which react with the substrate or substrates.

In another aspect, the invention also provides a process for identifying clones having a specified activity of interest by (i) generating one or more gene libraries derived from nucleic acid directly or indirectly isolated from a mixed population of organsims; and (ii) screening said exposed libraries utilizing an assay requiring a binding event or the covalent modification of a target, and a high throughput cell analyzer, e.g., a fluorescence activated cell sorter or non-optical cell sorter, to identify positive clones.

The invention further provides a method of screening for an agent that modulates the activity of a target protein or other cell component (e.g., nucleic acid), wherein the target and a selectable marker are expressed by a recombinant cell, by co-encapsulating the agent in a microenvironment with the recombinant cell expressing the target and detectable marker and detecting the effect of the agent on the activity of the target cell component.

In another embodiment, the invention provides a method for enriching for target DNA sequences containing at least a partial coding region for at least one specified activity in a DNA sample by co-encapsulating a mixture of target DNA obtained from a mixture of organisms with a mixture of DNA probes including a detectable marker and at least a portion of a DNA sequence encoding at least one enzyme having a specified enzyme activity and a detectable marker; incubating the co-encapsulated mixture under such conditions and for such time as to allow hybridization of complementary sequences and screening for the target DNA. Optionally the method further comprises transforming host cells with recovered target DNA to produce an expression library of a plurality of clones.

The invention further provides a method of screening for an agent that modulates the interaction of a first test protein linked to a DNA binding moiety and a second test protein linked to a transcriptional activation moiety by co-encapsulating the agent with the first test protein and second test protein in a suitable microenvironment and determining the ability of the agent to modulate the interaction of the first test protein linked to a DNA binding moiety with the second test protein covalently linked to a transcriptional activation moiety, wherein the agent enhances or inhibits the expression of a detectable protein.

In yet another aspect, the present invention provides a method for identifying a polynucleotide in a liquid phase, including contacting a plurality of polynucleotides derived from at least one organism, e.g., a mixed population of organisms, including microorganisms or plant tissue, with at least one nucleic acid probe under conditions that allow hybridization of the probe to the polynucleotides having complementary sequences, wherein the probe is labeled with a detectable molecule (e.g., a fluorescent, magnetic or other molecule). The detectable molecule changes, e.g., fluoresces, upon interaction of the probe to a target polynucleotide in the library. Clones from the library are then separated with an analyzer that detects the change in the detectable molecule, e.g., fluorescence, magnetic field or dielectric signature. The detectable molecule may also be a bioluminescent molecule, a chemiluminescent molecule, a colorimetric molecule, an electromagnetic molecule, an isotopic molecule, a thermal molecule or an enzymatic substrate. The separated clones can be contacted with a reporter system that identifies a polynucleotide encoding a polypeptide or a small molecule of interest, for example, and the clones capable of modulating expression or activity of the reporter system identified thereby identifying a polynucleotide of interest. The liquid phase of the embodiment includes in a solution (cell-free), in a cell, or in a non-solid phase.

In another embodiment, the invention provides a method for identifying a polynucleotide encoding a polypeptide of interest. The method includes co-encapsulating in a microenvironment a plurality of library clones containing DNA obtained from a mixed population of organisms with a mixture of oligonucleotide probes comprising a detectable marker and at least a portion of a polynucleotide sequence encoding a polypeptide of interest having a specified bioactivity. The encapsulated clones are incubated under such conditions and for such time as to allow interaction of complementary sequences and clones containing a complement to the oligonucleotide probe encoding the polypeptide of interest identified by separating clones with a fluorescent analyzer or non-optical analyzer that detects the detectable marker.

In yet another embodiment, the invention provides a method for high throughput screening of a polynucleotide library for a polynucleotide of interest that encodes a molecule of interest. The method includes contacting a library containing a plurality of clones comprising polynucleotides derived from a mixed population of organisms with a plurality of oligonucleotide probes labeled with a detectable molecule wherein said detectable molecule becomes detectable upon interaction of the probe to a target polynucleotide in the library; separating clones with an analyzer that detects the detectable marker; contacting the separated clones with a reporter system that identifies a polynucleotide encoding the molecule of interest; and identifying clones capable of modulating expression or activity of the reporter system thereby identifying a polynucleotide of interest.

In another embodiment, the invention provides a method of screening for a polynucleotide encoding an activity of interest. The method includes (a) obtaining polynucleotides from a sample containing a mixed population of organisms; (b) normalizing the polynucleotides obtained from the sample; (c) generating a library from the normalized polynucleotides; (d) contacting the library with a plurality of oligonucleotide probes comprising a detectable marker and at least a portion of a polynucleotide sequence encoding a polypeptide of interest having a specified activity to select library clones positive for a sequence of interest; (e) selecting clones with an analyzer (e.g. a fluorescent or non-optical analyzer) that detects the marker; (f) contacting the selected clones with a reporter system that identifies a polynucleotide encoding the activity of interest; and (g) identifying clones capable of modulating expression or activity of the reporter system thereby identifying a polynucleotide of interest; wherein the positive clones contain a polynucleotide sequence encoding an activity of interest which is capable of catalyzing the bioactive substrate.

In yet another embodiment, the present invention provides a method for screening polynucleotides, comprising contacting a library of polynucleotides derived from a mixed population of organism with a probe oligonucleotide labeled with a detectable molecule, which is detectable upon binding of the probe to a target polynucleotide of the library, to select library polynucleotides positive for a sequence of interest; separating library members that are positive for the sequence of interest with an analyzer that detects the molecule; expressing the selected polynucleotides to obtain polypeptides; contacting the polypeptides with a reporter system; and identifying polynucleotides encoding polypeptides capable of modulating expression or activity of the reporter system.

In another embodiment, the invention provides a method for obtaining an organism from a mixed population of organisms in a sample. The method includes encapsulating in a microenvironment at least one organism from the sample; incubating the encapsulated organism under such conditions and for such a time to allow the at least one microorganism to grow or proliferate; and sorting the encapsulated organism by flow cytometry to obtain an organism from the sample.

In another emodiment, the invention provides a method for identifying a polynucleotide in a liquid phase comprising:

a) contacting a plurality of polynucleotides derived from at least one organism with at least one nucleic acid probe under conditions that allow hybridization of the probe to the polynucleotides having complementary sequences, wherein the probe is labeled with a detectable molecule; and

b) identifying a polynucleotide of interest with an analyzer that detects the detectable molecule.

According to another embodiment of the invention, a sample screening apparatus includes a plurality of capillaries formed into an array of adjacent capillaries, wherein each capillary comprises at least one wall defining a lumen for retaining a sample. The apparatus further includes interstitial material disposed between adjacent capillaries in the array, and one or more reference indicia formed within of the interstitial material.

According to another embodiment of the invention, a capillary for screening a sample, wherein the capillary is adapted for being bound in an array of capillaries, includes a first wall defining a lumen for retaining the sample, and a second wall formed of a filtering material, for filtering excitation energy provided to the lumen to excite the sample.

According to yet another embodiment of the invention, a method for incubating a bioactivity or biomolecule of interest includes the steps of introducing a first component into at least a portion of a capillary of a capillary array, wherein each capillary of the capillary array comprises at least one wall defining a lumen for retaining the first component, and introducing an air bubble into the capillary behind the first component. The method further includes the step of introducing a second component into the capillary, wherein the second component is separated from the first component by the air bubble.

In yet another embodiment of the invention, a method of incubating a sample of interest includes introducing a first liquid labeled with a detectable particle into a capillary of a capillary array, wherein each capillary of the capillary array comprises at least one wall defining a lumen for retaining the first liquid and the detectable particle, and wherein the at least one wall is coated with a binding material for binding the detectable particle to the at least one wall. The method further includes removing the first liquid from the capillary tube, wherein the bound detectable particle is maintained within the capillary, and introducing a second liquid into the capillary tube.

Another embodiment of the invention includes a recovery apparatus for a sample screening system, wherein the system includes a plurality of capillaries formed into an array. The recovery apparatus includes a recovery tool adapted to contact at least one capillary of the capillary array and recover a sample from the at least one capillary. The recovery apparatus further includes an ejector, connected with the recovery tool, for ejecting the recovered sample from the recovery tool.

### BRIEF DESCRIPTION OF THE FIGURES

FIG. 1 illustrates the protocol used in the cell sorting method of the invention to screen for a polynucleotide of interest, in this case using a (library excised into E. coli). The clones of interest are isolated by sorting.

FIG. 2 shows a microtiter plate where clones or cells are sorted in accordance with the invention. Typically one cell or cells grown within a microdroplet are dispersed per well and grown up as clones.

FIG. 3 depicts a co-encapsulation assay. Cells containing library clones are coencapsulated with a substrate or labeled oligonucleotide. Encapsulation can occur in a variety of means, including GMDs, liposomes, and ghost cells. Cells are screened via high throughput screening on a fluorescence analyzer.

FIG. 4 depicts a side scatter versus forward scatter graph of FACS sorted gel-microdroplets (GMDs) containing a species of Streptomyces which forms unicells. Empty gel-microdroplets are distinguished from free cells and debris, also.

FIG. 5 is a depiction of a FACS/Biopanning method described herein and described in Example 3, below.

FIG. 6A shows an example of dimensions of a capillary array of the invention.

FIG. 6B illustrates an array of capillary arrays.

FIG. 7 shows a top cross-sectional view of a capillary array.

FIG. 8 is a schematic depicting the excitation of and emission from a sample within the capillary lumen according to one embodiment of the invention.

FIG. 9 is a schematic depicting the filtering of excitation and emission light to and from a sample within the capillary lumen according to an alternative embodiment of the invention.

FIG. 10 illustrates an embodiment of the invention in which a capillary array is wicked by contacting a sample containing cells, and humidified in a humidified incubator followed by imaging and recovery of cells in the capillary array.

FIG. 11 illustrates a method for incubating a sample in a capillary tube by an evaporative and capillary wicking cycle.

FIG. 12A shows a portion of a surface of a capillary array on which condensation has formed.

FIG. 12B shows the portion of the surface of the capillary array, depicted in FIG. 12A, in which the surface is coated with a hydrophobic layer to inhibit condensation near an end of individual capillaries.

FIGS. 13A-C depict a method of retaining at least two components within a capillary.

FIG. 14A depicts capillary tubes containing paramagnetic beads and cells.

FIG. 14B depicts the use of the paramagnetic beads to stir a sample in a capillary tube.

FIG. 15 depicts an excitation apparatus for a detection system according to an embodiment of the invention.

FIG. 16 illustrates a system for screening samples using a capillary array according to an embodiment of the invention.

FIG. 17A illustrates one example of a recovery technique useful for recovering a sample from a capillary array. In this depiction a needle is contacted with a capillary containing a sample to be obtained. A vacuum is created to evacuate the sample from the capillary tube and onto a filter.

FIG. 17B illustrates one sample recovery method in which the recovery device has an outer diameter greater than the inner diameter of the capillary from which a sample is being recovered.

FIG. 17C illustrates another sample recovery method in which the recovery device has an outer diameter approximately equal to or less than the inner diameter of the capillary.

FIG. 17D shows the further processing of the sample once evacuated from the capillary.

FIG. 18 is a schematic showing high throughput enrichment of low copy gene targets.

FIG. 19 is a schematic of FACS-Biopanning using high throughput culturing. Polyketide synthase sequences from environmental samples are shown in the alignment.

FIG. 20 shows whole cell hybridization for biopanning.

FIG. 21 is a schematic showing co-encapsulation of a eukaryotic cell and a bacterial cell.

FIG. 22 shows a whole cell hybridization schematic for biopanning and FACS sorting.

FIG. 23 shows a schematic of T7 RNA Polymerase Expression system.

### DETAILED DESCRIPTION OF THE INVENTION

The present invention provides a method for rapid sorting and screening of libraries derived from a mixed population of organisms from, for example, an environmental sample or an uncultivated population of organisms. In one embodiment, gene libraries are generated, clones are either exposed to a substrate or substrate(s) of interest, or hybridized to a fluorescence labeled probe having a sequence corresponding to a sequence of interest and positive clones are identified and isolated via fluorescence activated cell sorting. Cells can be viable or non-viable during the process or at the end of the process, as nucleic acids encoding a positive activity can be isolated and cloned utilizing techniques well known in the art.

This invention differs from fluorescence activated cell sorting, as normally performed, in several aspects. Previously, FACS machines have been employed in studies focused on the analyses of eukaryotic and prokaryotic cell lines and cell culture processes. FACS has also been utilized to monitor production of foreign proteins in both eukaryotes and prokaryotes to study, for example, differential gene expression. The detection and counting capabilities of the FACS system have been applied in these examples. However, FACS has never previously been employed in a discovery process to screen for and recover bioactivities in prokaryotes. In addition, non-optical methods have not been used to identify or discover novel bioactivities or biomolecules. Furthermore, the present invention does not require cells to survive, as do previously described technologies, since the desired nucleic acid (recombinant clones) can be obtained from alive or dead cells. For example, the cells only need to be viable long enough to contain, carry or synthesize a complementary nucleic acid sequence to be detected, and can thereafter be either viable or non-viable cells so long as the complementary sequence remains intact. The present invention also solves problems that would have been associated with detection and sorting of E. coli expressing recombinant enzymes, and recovering encoding nucleic acids. The invention includes within its embodiments apparatus capable of detecting a molecule or marker that is indicative of a bioactivity or biomolecule of interest, including optical and non-optical apparatus. In one embodiment, the present invention includes within its embodiments any apparatus capable of detecting fluorescent wavelengths associated with biological material, such apparatuses are defined herein as fluorescent analyzers (one example of which is a FACS apparatus).

The use of a culture-independent approach to directly clone genes encoding novel enzymes from, for example, an environmental sample containing a mixed population of organisms allows one to access untapped resources of biodiversity. In one embodiment, the invention is based on the construction of “mixed population libraries” which represent the collective genomes of naturally occurring organisms archived in cloning vectors that can be propagated in suitable prokaryotic hosts. Because the cloned DNA is initially extracted directly from environmental samples, the libraries are not limited to the small fraction of prokaryotes that can be grown in pure culture. Additionally, a normalization of the DNA present in these samples could allow more equal representation of the DNA from all of the species present in the original sample. This can increase the efficiency of finding interesting genes from minor constituents of the sample which may be under-represented by several orders of magnitude compared to the dominant species.

Prior to the present invention, the evaluation of complex mixed population expression libraries was rate limiting. The present invention allows the rapid screening of complex mixed population libraries, containing, for example, genes from thousands of different organisms. The benefits of the present invention can be seen, for example, in screening a complex mixed population sample. Screening of a complex sample previously required one to use labor intensive methods to screen several million clones to cover the genomic biodiversity. The invention represents an extremely high-throughput screening method which allows one to assess this enormous number of clones. The method disclosed herein allows the screening anywhere from about 30 million to about 200 million clones per hour for a desired nucleic acid sequence or biological activity. This allows the thorough screening of mixed population libraries for clones expressing novel biomolecules.

The invention provides methods and composition whereby one can screen, sort or identify a polynucleotide sequence, polypeptide, or molecule of interest from a mixed population of organisms (e.g., organisms present in a mixed population sample) based on polynucleotide sequences present in the sample. Thus, the invention provides methods and compositions useful in screening organisms for a desired biological activity or biological sequence and to assist in obtaining sequences of interest that can further be used in directed evolution, molecular biology, biotechnology and industrial applications. By screening and identifying the nucleic acid sequences present in the sample, the invention increases the repertoire of available sequences that can be used for the development of diagnostics, therapeutics or molecules for industrial applications. Accordingly, the methods of the invention can identify novel nucleic acid sequences encoding proteins or polypeptides having a desired biological activity.

In one embodiment, the invention provides a method for high throughput culturing of organisms. In one aspect, the organisms are a mixed population of organisms. In another aspect, the organisms include host cells of a library containing nucleic acids. For example, such libraries include nucleic acid obtained from various isolates of organisms, which are then pooled; nucleic acid obtained from isolate libraries, which are then pooled; or nucleic acids derived directly from a mixed population of organisms. Generally, a sample containing the organisms is mixed with a composition that can form a microenvironment, as described herein, e.g., a gel microdroplet or a liposome. In one aspect, as illustrated in Example 8 a mixed population of microorganisms is mixed with the encapsulation material in such a way that preferably fewer than 5 microorganisms are encapsulated. Preferably, only one microorganism is encapsulated in each microenvironment system.

Once encapsulated, the cells are cultured in a manner which allows growth of the organisms, e.g., host cells of a library. For example, Example 8 provides growth of the encapsulated organisms in a chromatography column which allows a flow of growth medium providing nutrients for growth and for removal of waste products from cells. Over a period of time (20 minutes to several weeks or months), a clonal population of the preferably one organism grows within the microenvironment.

After a desired period of time, microenvironments, e.g., gel microdroplets, can be sorted to eliminate “empty” microenvironments and to sort for the occupied microenvironments. The nucleic acid from organisms in the sorted microenvironments can be studied directly, for example, by treating with a PCR mixture and amplified immediately after sorting. In one Example described herein, 16S rRNA genes from individual cells were studied and organisms assessed for phylogenetic diversity from the samples.

In another aspect, the high throughput culturing methods of the invention allow culturing of organisms and enrichment of low copy gene targets. For example, a library of nucleic acid obtained from various isolates of organisms, which are then pooled; nucleic acid obtained from isolate libraries, which are then pooled; or nucleic acids derived directly from a mixed population of organisms, for example, are encapsulated, e.g., in a gel microdroplet or other microenvironment, and grown under conditions which allow clonal expansion of each organism in the microenvironment. In one aspect, the cells of the clonal population are lysed and treated with proteinases to yield nucleic acid (see Figure X) (e.g., the microcolonies are deproteinized by incubating gel microdroplets in lysis solution containing proteinase K at 37 degrees C. for 30 minutes). In order to denature and neutralize nucleic acid entrapped in the microenvironments, they are denatured with alkaline denaturing solution (0.5M NaOH) and neutralized (e.g., with Tris pH8). In one particular example, nucleic acid entrapped in the microenvironment is hybridized with Digoxiginin (DIG)-labeled oligonucleotides (30-50 nt) in Dig Easy Hyb (available from Roche) overnight at 37 degrees C., followed by washing with 0.3×SSC and 0.1×SSC at 38-50 degrees C. to achieve desired stringency. One of skill in the art will appreciate that this is merely an example and not meant to limit the invention in any way. For example, other labels commonly used in the art, e.g., fluorescent labels such as GFP or chemiluminescent labels, can be utilized in the invention methods.

The nucleic acid is hybridized with a probe which is preferably labeled. A signal can be amplified with a secondary label (e.g., fluorescent) and the nucleic acid sorted for fluorescent microenvironments, e.g., gel microdroplets. Nucleic acid that is fluorescent can be isolated and further studied or cloned into a host cell for further manipulation. In one particular example, signals are amplified with Tyramide Signal Amplification (TSA) kit from Molecular Probe. TSA is an enzyme-mediated signal amplification method that utilizes horseradish peroxidase (HRP) to depose fluorogenic tyramide molecules and generate high-density labeling of a target nucleic acid sequence in situ. The signal amplification is conferred by the turnover of multiple tyramide substrates per HRP molecule, and increases in signal strength of over 1,000-fold have been reported. The procedure involves incubating GMDs with anti-DIG conjugated horseradish peroxidase (anti-DIG-HRP) (Roche, Ind.) for 3 hours at room temperature. Then the tyramide substrate solution will be added and incubated for 30 minutes at room temperature.

In one aspect, this high throughput culturing method followed by sorting (e.g., FACS) screening (e.g., biopanning), allows for identification of gene targets. It may be desirable to screen for nucleic acids encoding virtually any protein or any bioactivity and to compare such nucleic acids among various species of organisms in a sample (e.g., study polyketide sequences from a mixed population). In another aspect, nucleic acid derived from high throughput culturing of organisms can be obtained for further study or for generation of a library. Such nucleic acid can be pooled and a library created, or alternatively, individual libraries from clonal populations of organisms can be generated and then nucleic acid pooled from those libraries to generate a more complex library. The libraries generated as described herein can be utilized for the discovery of biomolecules (e.g., nucleic acid or bioactivities) or for evolving nucleic acid molecules identified by the high throughput culturing methods described in the present invention invention. Such evolution methods are known in the art or described herein, such as, shuffling, cassette mutagenesis, recursive ensemble mutagenesis, sexual PCR, directed evolution, exonuclease-mediated reassembly, codon site-saturation mutagenesis, amino acid site-saturation mutagenesis, gene site saturation mutagenesis, introduction of mutations by non-stochastic polynucleotide reassembly methods, synthetic ligation polynucleotide reassembly, gene reassembly, oligonucleotide-directed saturation mutagenesis, in vivo reassortment of polynucleotide sequences having partial homology, naturally occurring recombination processes which reduce sequence complexity, and any combination thereof.

Flow cytometry has been used in cloning and selection of variants from existing cell clones. This selection, however, has required stains that diffuse through cells passively, rapidly and irreversibly, with no toxic effects or other influences on metabolic or physiological processes. Since, typically, flow sorting has been used to study animal cell culture performance, physiological state of cells, and the cell cycle, one goal of cell sorting has been to keep the cells viable during and after sorting.

There currently are no reports in the literature of screening and discovery of polynucleotide sequence in libraries by cell sorting based on fluorescence (e.g. fluorescent activated cell sorting), or non-optical markers (e.g., magnetic fields and the like). Furthermore there are no reports of recovering DNA encoding bioactivities screened by FACS or non-optical techniques and additionally screening for a bioactivity of interest. The present invention provides these methods to allow the extremely rapid screening of viable or non-viable cells to recover desirable activities and the nucleic acid encoding those activities.

Fluorescence and other forms of staining have been employed for microbial discrimination and identification, and in the analysis of the interaction of drugs and antibiotics with microbial cells. Flow cytometry has been used in aquatic biology, where autofluorescence of photosynthetic pigments are used in the identification of algae or DNA stains are used to quantify and count marine populations (Davey and Kell, 1996). Diaper and Edwards used flow cytometry to detect viable bacteria after staining with a range of fluorogenic esters including fluorescein diacetate (FDA) derivatives and CemChrome B, a stain sold commercially for the detection of viable bacteria in suspension (Diaper and Edwards, 1994). Labeled antibodies and oligonucleotide probes can also been used for these purposes.

Papers have been published describing the application of flow cytometry to the detection of native and recombinant enzymatic activities in eukaryotes. Betz et al. studied native (non-recombinant) lipase production by the eukaryote, Rhizopus arrhizus with flow cytometry. They found that spore suspensions of the mold were heterogeneous as judged by light-scattering data obtained with excitation at 633 nm, and they sorted clones of the subpopulations into the wells of microtiter plates. After germination and growth, lipase production was automatically assayed (turbidimetrically) in the microtiter plates, and a representative set of the most active were reisolated, cultured, and assayed conventionally (Betz et al., 1984). The ability of flow cytometry to make measurements on single cells means that individual cells with high levels of expression (e.g., due to gene amplification or higher plasmid copy number) could be detected.

Cells with chromogenic or fluorogenic substrates yield colored and fluorescent products, respectively. Previously, it had been thought that the flow cytometry-fluorescence activated cell sorter approaches could be of benefit only for the analysis of cells that contain intracellularly, or are normally physically associated with, the enzymatic activity of a molecule of interest. On this basis, one could only use fluorogenic reagents which could penetrate the cell and which are thus potentially cytotoxic. In addition, gel microdroplets (GMDs) can be used during FACS sorting and culturing. The use of GMDs containing (physically) single cells which can take up nutrients, secrete products, and grow to form colonies is useful in the present invention. The diffusional properties of GMDs may be made such that sufficient extracellular product remains associated with each individual GMD, so as to permit flow cytometric analysis and cell sorting on the basis of concentration of secreted molecule within each microdroplet. Beads have also been used to isolate mutants growing at different rates, and to analyze antibody secretion by hybridoma cells and the nutrient sensitivity of hybridoma cells.

The GMD technology has had significance in amplifying the signals available in flow cytometric analysis, and in permitting the screening and sorting of microbial strains in strain improvement and isolation programs. GMD or other related technologies can be used in the present invention to localize, sort as well as amplify signals in the high throughput screening of recombinant libraries. Cell viability during the screening is not an issue or concern since nucleic acid can be recovered from the microdroplet.

Different types of encapsulation strategies and compounds or polymers can be used with the present invention. For instance, high temperature agaroses can be employed for making microdroplets stable at high temperatures, allowing stable encapsulation of cells subsequent to heat-kill steps utilized to remove all background activities when screening for thermostable bioactivities. Encapsulation can be in beads, high temperature agaroses, gel microdroplets, cells, such as ghost red blood cells or macrophages, liposomes, or any other means of encapsulating and localizing molecules.

For example, methods of preparing liposomes have been described (i.e., U.S. Pat. Nos. 5,653,996, 5,393,530 and 5,651,981), as well as the use of liposomes to encapsulate a variety of molecules U.S. Pat. Nos. 5,595,756, 5,605,703, 5,627,159, 5,652,225, 5,567,433, 4,235,871, 5,227,170). Entrapment of proteins, viruses, bacteria and DNA in erythrocytes during endocytosis has been described, as well (Journal of Applied Biochemistry 4, 418-435 (1982)). Erythrocytes employed as carriers in vitro or in vivo for substances entrapped during hypo-osmotic lysis or dielectric breakdown of the membrane have also been described (reviewed in Ihler, G. M. (1983) J. Pharm. Ther). These techniques are useful in the present invention to encapsulate samples for screening.

“Microenvironment”, as used herein, is any molecular structure which provides an appropriate environment for facilitating the interactions necessary for the method of the invention. An environment suitable for facilitating molecular interactions include, for example, gel microdroplets, ghost cells, macrophages or liposomes. Liposomes can be prepared from a variety of lipids including phospholipids, glycolipids, steroids, long-chain alkyl esters; e.g., alkyl phosphates, fatty acid esters; e.g., lecithin, fatty amines and the like. A mixture of fatty material may be employed such a combination of neutral steroid, a charge amphiphile and a phospholipid. Illustrative examples of phospholipids include lecithin, sphingomyelin and dipalmitoylphos-phatidylcholine. Representative steroids include cholesterol, cholestanol and lanosterol. Representative charged amphiphilic compounds generally contain from 12-30 carbon atoms. Mono- or dialkyl phosphate esters, or alkyl amines; e.g., dicetyl phosphate, stearyl amine, hexadecyl amine, dilauryl phosphate, and the like.

The invention methods include a system and method for holding and screening samples. According to one embodiment of the invention, a sample screening apparatus includes a plurality of capillaries formed into an array of adjacent capillaries, wherein each capillary comprises at least one wall defining a lumen for retaining a sample. The apparatus further includes interstitial material disposed between adjacent capillaries in the array, and one or more reference indicia formed within of the interstitial material. (see co-pending applications Ser. No. 09/687,219 and 09/894,956, herein incorporated by reference in their entirety).

According to another embodiment of the invention, a capillary for screening a sample, wherein the capillary is adapted for being bound in an array of capillaries, includes a first wall defining a lumen for retaining the sample, and a second wall formed of a filtering material, for filtering excitation energy provided to the lumen to excite the sample.

According to yet another embodiment of the invention, a method for incubating a bioactivity or biomolecule of interest includes the steps of introducing a first component into at least a portion of a capillary of a capillary array, wherein each capillary of the capillary array comprises at least one wall defining a lumen for retaining the first component, and introducing an air bubble into the capillary behind the first component. The method further includes the step of introducing a second component into the capillary, wherein the second component is separated from the first component by the air bubble.

In yet another embodiment of the invention, a method of incubating a sample of interest includes introducing a first liquid labeled with a detectable particle into a capillary of a capillary array, wherein each capillary of the capillary array comprises at least one wall defining a lumen for retaining the first liquid and the detectable particle, and wherein the at least one wall is coated with a binding material for binding the detectable particle to the at least one wall. The method further includes removing the first liquid from the capillary tube, wherein the bound detectable particle is maintained within the capillary, and introducing a second liquid into the capillary tube.

Another embodiment of the invention includes a recovery apparatus for a sample screening system, wherein the system includes a plurality of capillaries formed into an array. The recovery apparatus includes a recovery tool adapted to contact at least one capillary of the capillary array and recover a sample from the at least one capillary. The recovery apparatus further includes an ejector, connected with the recovery tool, for ejecting the recovered sample from the recovery tool.

As used herein and in the appended claims, the singular forms “a,” “and,” and “the” include plural referents unless the context clearly dictates otherwise. Thus, for example, reference to “a clone” includes a plurality of clones and reference to “the nucleic acid sequence” generally includes reference to one or more nucleic acid sequences and equivalents thereof known to those skilled in the art, and so forth.

Unless defined otherwise, all technical and scientific terms used herein have the same meaning as commonly understood to one of ordinary skill in the art to which the invention belongs. Although any methods, devices and materials similar or equivalent to those described herein can be used in the practice or testing of the invention, the preferred methods, devices and materials are now described.

All publications mentioned herein are incorporated herein by reference in full for the purpose of describing and disclosing the databases, proteins, and methodologies, which are described in the publications which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

An “amino acid” is a molecule having the structure wherein a central carbon atom (the β-carbon atom) is linked to a hydrogen atom, a carboxylic acid group (the carbon atom of which is referred to herein as a “carboxyl carbon atom”), an amino group (the nitrogen atom of which is referred to herein as an “amino nitrogen atom”), and a side chain group, R. When incorporated into a peptide, polypeptide, or protein, an amino acid loses one or more atoms of its amino acid carboxylic groups in the dehydration reaction that links one amino acid to another. As a result, when incorporated into a protein, an amino acid is referred to as an “amino acid residue.”

“Protein” or “polypeptide” refers to any polymer of two or more individual amino acids (whether or not naturally occurring) linked via a peptide bond, and occurs when the carboxyl carbon atom of the carboxylic acid group bonded to the β-carbon of one amino acid (or amino acid residue) becomes covalently bound to the amino nitrogen atom of amino group bonded to the p-carbon of an adjacent amino acid. The term “protein” is understood to include the terms “polypeptide” and “peptide” (which, at times may be used interchangeably herein) within its meaning. In addition, proteins comprising multiple polypeptide subunits (e.g., DNA polymerase II, RNA polymerase II) or other components (for example, an RNA molecule, as occurs in telomerase) will also be understood to be included within the meaning of “protein” as used herein. Similarly, fragments of proteins and polypeptides are also within the scope of the invention and may be referred to herein as “proteins.” A particular amino acid sequence of a given protein (i.e., the polypeptide's “primary structure,” when written from the amino-terminus to carboxy-terminus) is determined by the nucleotide sequence of the coding portion of a MRNA, which is in turn specified by genetic information, typically genomic DNA (including organelle DNA, e.g., mitochondrial or chloroplast DNA). Thus, determining the sequence of a gene assists in predicting the primary sequence of a corresponding polypeptide and more particular the role or activity of the polypeptide or proteins encoded by that gene or polynucleotide sequence.

The term “isolated” means altered “by the hand of man” from its natural state; i.e., if it occurs in nature, it has been changed or removed from its original environment, or both. For example, a naturally occurring polynucleotide or a polypeptide naturally present in a living animal, a biological sample or an environmental sample in its natural state is not “isolated”, but the same polynucleotide or polypeptide separated from the coexisting materials of its natural state is “isolated”, as the term is employed herein. Such polynucleotides, when introduced into host cells in culture or in whole organisms, still would be isolated, as the term is used herein, because they would not be in their naturally occurring form or environment. Similarly, the polynucleotides and polypeptides may occur in a composition, such as a media formulation (solutions for introduction of polynucleotides or polypeptides, for example, into cells or compositions or solutions for chemical or enzymatic reactions).

“Polynucleotide” or “nucleic acid sequence” refers to a polymeric form of nucleotides. In some instances a polynucleotide refers to a sequence that is not immediately contiguous with either of the coding sequences with which it is immediately contiguous (one on the 5′ end and one on the 3′ end) in the naturally occurring genome of the organism from which it is derived. The term therefore includes, for example, a recombinant DNA which is incorporated into a vector; into an autonomously replicating plasmid or virus; or into the genomic DNA of a prokaryote or eukaryote, or which exists as a separate molecule (e.g., a cDNA) independent of other sequences. The nucleotides of the invention can be ribonucleotides, deoxyribonucleotides, or modified forms of either nucleotide. A polynucleotides as used herein refers to, among others, single-and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions.

In addition, polynucleotide as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The term polynucleotide encompasses genomic DNA or RNA (depending upon the organism, i.e., RNA genome of viruses), as well as mRNA encoded by the genomic DNA, and cDNA.

As mentioned above, there is currently a need in the biotechnology and chemical industry for molecules that can optimally carry out biological or chemical processes (e.g., enzymes). Identifying novel enzymes in a mixed population environmental sample is one solution to this problem. By rapidly identifying polypeptides having an activity of interest and polynucleotides encoding the polypeptide of interest the invention provides methods, compositions and sources for the development of biologics, diagnostics, therapeutics, and compositions for industrial applications.

All classes of molecules and compounds that are utilized in both established and emerging chemical, pharmaceutical, textile, food and feed, detergent markets must meet economical and environmental standards. The synthesis of polymers, pharmaceuticals, natural products and agrochemicals is often hampered by expensive processes which produce harmful byproducts and which suffer from poor or inefficient catalysis. Enzymes, for example, have a number of remarkable advantages which can overcome these problems in catalysis: they act on single functional groups, they distinguish between similar functional groups on a single molecule, and they distinguish between enantiomers. Moreover, they are biodegradable and function at very low mole fractions in reaction mixtures. Because of their chemo-, regio- and stereospecificity, enzymes present a unique opportunity to optimally achieve desired selective transformations. These are often extremely difficult to duplicate chemically, especially in single-step reactions. The elimination of the need for protection groups, selectivity, the ability to carry out multi-step transformations in a single reaction vessel, along with the concomitant reduction in environmental burden, has led to the increased demand for enzymes in chemical and pharmaceutical industries. Enzyme-based processes have been gradually replacing many conventional chemical-based methods. A current limitation to more widespread industrial use is primarily due to the relatively small number of commercially available enzymes. Only ˜300 enzymes (excluding DNA modifying enzymes) are at present commercially available from the >3000 non DNA-modifying enzyme activities thus far described.

The use of enzymes for technological applications also may require performance under demanding industrial conditions. This includes activities in environments or on substrates for which the currently known arsenal of enzymes was not evolutionarily selected. However, the natural environment provides extreme conditions including, for example, extremes in temperature and pH. A number of organisms have adapted to these conditions due in part to selection for polypeptides than can withstand these extremes.

Enzymes have evolved by selective pressure to perform very specific biological functions within the milieu of a living organism, under conditions of temperature, pH and salt concentration. For the most part, the non-DNA modifying enzyme activities thus far described have been isolated from mesophilic organisms, which represent a very small fraction of the available phylogenetic diversity. The dynamic field of biocatalysis takes on a new dimension with the help of enzymes isolated from microorganisms that thrive in extreme environments. For example, such enzymes must function at temperatures above 100° C. in terrestrial hot springs and deep sea thermal vents, at temperatures below 0° C. in arctic waters, in the saturated salt environment of the Dead Sea, at pH values around 0 in coal deposits and geothermal sulfur-rich springs, or at pH values greater than 11 in sewage sludge. Environmental samples obtained, for example, from extreme conditions containing organisms, polynucleotides or polypeptides (e.g., enzymes) open a new field in biocatalysis. By rapidly screening for polynucleotides encoding polypeptides of interest, the invention provides not only a source of materials for the development of biologics, therapeutics, and enzymes for industrial applications, but also provides a new materials for further processing by, for example, directed evolution and mutagenesis to develop molecules or polypeptides modified for particular activity or conditions.

In addition to the need for new enzymes for industrial use, there has been a dramatic increase in the need for bioactive compounds with novel activities. This demand has arisen largely from changes in worldwide demographics coupled with the clear and increasing trend in the number of pathogenic organisms that are resistant to currently available antibiotics. For example, while there has been a surge in demand for antibacterial drugs in emerging nations with young populations, countries with aging populations, such as the U.S., require a growing repertoire of drugs against cancer, diabetes, arthritis and other debilitating conditions. The death rate from infectious diseases has increased 58% between 1980 and 1992 and it has been estimated that the emergence of antibiotic resistant microbes has added in excess of $30 billion annually to the cost of health care in the U.S. alone. (Adams et al., Chemical and Engineering News, 1995; Amann et al., Microbiological Reviews, 59, 1995). As a response to this trend pharmaceutical companies have significantly increased their screening of microbial diversity for compounds with unique activities or specificity. Accordingly, the invention can be used to obtain and identify polynucleotides and related sequence specific information from, for example, infectious microorganisms present in the environment such as, for example, in the gut of various macroorganisms.

In another embodiment, the methods and compositions of the invention provide for the identification of lead drug compounds present in an environmental sample. The methods of the invention provide the ability to mine the environment for novel drugs or identify related drugs contained in different microorganisms. There are several common sources of lead compounds (drug candidates), including natural product collections, synthetic chemical collections, and synthetic combinatorial chemical libraries, such as nucleotides, peptides, or other polymeric molecules that have been identified or developed as a result of environmental mining. Each of these sources has advantages and disadvantages. The success of programs to screen these candidates depends largely on the number of compounds entering the programs, and pharmaceutical companies have to date screened hundred of thousands of synthetic and natural compounds in search of lead compounds. Unfortunately, the ratio of novel to previously-discovered compounds has diminished with time. The discovery rate of novel lead compounds has not kept pace with demand despite the best efforts of pharmaceutical companies. There exists a strong need for accessing new sources of potential drug candidates. Accordingly, the invention provides a rapid and efficient method to identify and characterize environmental samples that may contain novel drug compounds.

The majority of bioactive compounds currently in use are derived from soil microorganisms. Many microbes inhabiting soils and other complex ecological communities produce a variety of compounds that increase their ability to survive and proliferate. These compounds are generally thought to be nonessential for growth of the organism and are synthesized with the aid of genes involved in intermediary metabolism hence their name—“secondary metabolites”. Secondary metabolites are generally the products of complex biosynthetic pathways and are usually derived from common cellular precursors. Secondary metabolites that influence the growth or survival of other organisms are known as “bioactive” compounds and serve as key components of the chemical defense arsenal of both micro- and macro-organisms. Humans have exploited these compounds for use as antibiotics, antiinfectives and other bioactive compounds with activity against a broad range of prokaryotic and eukaryotic pathogens. Approximately 6,000 bioactive compounds of microbial origin have been characterized, with more than 60% produced by the gram positive soil bacteria of the genus Streptomyces. (Barnes et al., Proc.Nat. Acad. Sci. U.S.A., 91, 1994). Of these, at least 70 are currently used for biomedical and agricultural applications. The largest class of bioactive compounds, the polyketides, include a broad range of antibiotics, immunosuppressants and anticancer agents which together account for sales of over $5 billion per year.

Despite the seemingly large number of available bioactive compounds, it is clear that one of the greatest challenges facing modem biomedical science is the proliferation of antibiotic resistant pathogens. Because of their short generation time and ability to readily exchange genetic information, pathogenic microbes have rapidly evolved and disseminated resistance mechanisms against virtually all classes of antibiotic compounds. For example, there are virulent strains of the human pathogens Staphylococcus and Streptococcus that can now be treated with but a single antibiotic, vancomycin, and resistance to this compound will require only the transfer of a single gene, vanA, from resistant Enterococcus species for this to occur. (Bateson et al., System. Appl. Microbiol, 12, 1989). When this crucial need for novel antibacterial compounds is superimposed on the growing demand for enzyme inhibitors, immunosuppressants and anti-cancer agents it becomes readily apparent why pharmaceutical companies have stepped up their screening of microbial samples for bioactive compounds.

The invention provides methods of identifying a nucleic acid sequence encoding a polypeptide having either known or unknown function. For example, much of the diversity in microbial genomes results from the rearrangement of gene clusters in the genome of microorganisms. These gene clusters can be present across species or phylogenetically related with other organisms.

For example, bacteria and many eukaryotes have a coordinated mechanism for regulating genes whose products are involved in related processes. The genes are clustered, in structures referred to as “gene clusters,” on a single chromosome and are transcribed together under the control of a single regulatory sequence, including a single promoter which initiates transcription of the entire cluster. The gene cluster, the promoter, and additional sequences that function in regulation altogether are referred to as an “operon” and can include up to 20 or more genes, usually from 2 to 6 genes. Thus, a gene cluster is a group of adjacent genes that are either identical or related, usually as to their function. Gene clusters are generally 15 kb to greater than 120 kb in length.

Some gene families consist of identical members. Clustering is a prerequisite for maintaining identity between genes, although clustered genes are not necessarily identical. Gene clusters range from extremes where a duplication is generated to adjacent related genes to cases where hundreds of identical genes lie in a tandem array. Sometimes no significance is discernable in a repetition of a particular gene. A principal example of this is the expressed duplicate insulin genes in some species, whereas a single insulin gene is adequate in other mammalian species.

Further, gene clusters undergo continual reorganization and, thus, the ability to create heterogeneous libraries of gene clusters from, for example, bacterial or other prokaryote sources is valuable in determining sources of novel proteins, particularly including enzymes such as, for example, the polyketide synthases that are responsible for the synthesis of polyketides having a vast array of useful activities. Other types of proteins that are the product(s) of gene clusters are also contemplated, including, for example, antibiotics, antivirals, antitumor agents and regulatory proteins, such as insulin.

As an example, polyketide synthases enzymes fall in a gene cluster. Polyketides are molecules which are an extremely rich source of bioactivities, including antibiotics (such as tetracyclines and erythromycin), anti-cancer agents (daunomycin), immunosuppressants (FK506 and rapamycin), and veterinary products (monensin). Many polyketides (produced by polyketide synthases) are valuable as therapeutic agents. Polyketide synthases are multifunctional enzymes that catalyze the biosynthesis of a huge variety of carbon chains differing in length and patterns of functionality and cyclization. Polyketide synthase genes fall into gene clusters and at least one type (designated type I) of polyketide synthases have large size genes and enzymes, complicating genetic manipulation and in vitro studies of these genes/proteins.

The ability to select and combine desired components from a library of polyketides and postpolyketide biosynthesis genes for generation of novel polyketides for study is appealing. The method(s) of the present invention make it possible to, and facilitate the cloning of, novel polyketide synthases, since one can generate gene banks with clones containing large inserts (especially when using the f-factor based vectors), which facilitates cloning of gene clusters.

Other biosynthetic genes include NRPS, glycosyl transferases and p450s.

For example, a gene cluster can be ligated into a vector containing an expression regulatory sequences which can control and regulate the production of a detectable protein or protein-related array activity from the ligated gene clusters. Use of vectors which have an exceptionally large capacity for exogenous nucleic acid introduction are particularly appropriate for use with such gene clusters and are described by way of example herein to include artificial chromosome vectors, cosmids, and the f-factor (or fertility factor) of E. coli. For example, the f-factor of E. coli is a plasmid which affects high-frequency transfer of itself during conjugation and is ideal to achieve and stably propagate large nucleic acid fragments, such as gene clusters from samples of mixed populations of organisms.

The nucleic acid isolated or derived from these samples (e.g., a mixed population of microorganisms) can preferably be inserted into a vector or a plasmid prior to screening of the polynucleotides. Such vectors or plasmids are typically those containing expression regulatory sequences, including promoters, enhancers and the like.

Accordingly, the invention provides novel systems to clone and screen mixed populations of organisms present, for example, in an environmental samples, for polynucleotides of interest, enzymatic activities and bioactivities of interest in vitro. The method(s) of the invention allow the cloning and discovery of novel bioactive molecules in vitro, and in particular novel bioactive molecules derived from uncultivated or cultivated samples. Large size gene clusters, genes and gene fragments can be cloned, sequenced and screened using the method(s) of the invention. Unlike previous strategies, the method(s) of the invention allow one to clone, screen and identify polynucleotides and the polypeptides encoded by these polynucleotides in vitro from a wide range of mixed population samples.

The invention allows one to screen for and identify polynucleotide sequences from complex mixed population samples. DNA libraries obtained from these samples can be created from cell free samples, so long as the sample contains nucleic acid sequences, or from samples containing cellular organisms or viral particles. The organisms from which the libraries may be prepared include prokaryotic microorganisms, such as Eubacteria and Archaebacteria, lower eukaryotic microorganisms such as fungi, algae and protozoa, as well as plants, plant spores and pollen. The organisms may be cultured organisms or uncultured organisms obtained from mixed population environmental samples and includes extremophiles, such as thermophiles, hyperthermophiles, psychrophiles and psychrotrophs.

Sources of nucleic acids used to construct a DNA library can be obtained from mixed population samples, such as, but not limited to, microbial samples obtained from Arctic and Antarctic ice, water or permafrost sources, materials of volcanic origin, materials from soil or plant sources in tropical areas, droppings from various organisms including mammals, invertebrates, as well as dead and decaying matter etc. Thus, for example, nucleic acids may be recovered from either a cultured or non-cultured organism and used to produce an appropriate DNA library (e.g., a recombinant expression library) for subsequent determination of the identity of the particular polynucleotide sequence or screening for bioactivity.

The following outlines a general procedure for producing libraries from both culturable and non-culturable organisms as well as mixed population of organisms, which libraries can be probed, sequenced or screened to select therefrom nucleic acid sequences having an identified, desired or predicted biological activity (e.g., an enzymatic activity or a small molecule).

As used herein a mixed population sample is any sample containing organisms or polynucleotides or a combination thereof, which can be obtained from any number of sources (as described above), including, for example, insect feces, soil, water, etc. Any source of nucleic acids in purified or non-purified form can be utilized as starting material. Thus, the nucleic acids may be obtained from any source which is contaminated by an organism or from any sample containing cells. The mixed population sample can be an extract from any bodily sample such as blood, urine, spinal fluid, tissue, vaginal swab, stool, amniotic fluid or buccal mouthwash from any mammalian organism. For non-mammalian (e.g., invertebrates) organisms the sample can be a tissue sample, salivary sample, fecal material or material in the digestive tract of the organism. An environmental sample also includes samples obtained from extreme environments including, for example, hot sulfur pools, volcanic vents, and frozen tundra. In addition, the sample can come from a variety of sources. For example, in horticulture and agricultural testing the sample can be a plant, fertilizer, soil, liquid or other horticultural or agricultural product; in food testing the sample can be fresh food or processed food (for example infant formula, seafood, fresh produce and packaged food); and in environmental testing the sample can be liquid, soil, sewage treatment, sludge and any other sample in the environment which is considered or suspected of containing an organism or polynucleotides.

When the sample is a mixture of material (e.g., a mixed population of organisms), for example, blood, soil and sludge, it can be treated with an appropriate reagent which is effective to open the cells and expose or separate the strands of nucleic acids. Mixed populations can comprise pools of cultured organisms or samples. For example, samples of organisms can be cultured prior to analysis in order to purify a particular population and thus obtaining a purer sample. Organisms, such as actinomycetes or myxobacteria, known to produce bioacitivities of interest can be enriched for, via culturing. Culturing of organisms in the sample can include culturing the organisms in microdroplets and separating the cultured microdroplets with a cell sorter into individual wells of a multi-well tissue culture plate from which further processing may be performed.

Accordingly, the sample comprises nucleic acids from, for example, a diverse and mixed population of organisms (e.g., microorganisms present in the gut of an insect). Nucleic acids are isolated from the sample using any number of methods for DNA and RNA isolation. Such nucleic acid isolation methods are commonly performed in the art. Where the nucleic acid is RNA, the RNA can be reversed transcribed to DNA using primers known in the art. Where the DNA is genomic DNA, the DNA can be sheared using, for example, a 25 gauge needle.

The nucleic acids are then cloned into avector. Cloning techniques are known in the art or can be developed by one skilled in the art, without undue experimentation. Vectors used in the present invention include: plasmids, phages, cosmids, phagemids, viruses (e.g., retroviruses, parainfluenzavirus, herpesviruses, reoviruses, paramyxoviruses, and the like), artificial chromosomes, or selected portions thereof (e.g., coat protein, spike glycoprotein, capsid protein). For example, cosmids and phagemids are typically used where the specific nucleic acid sequence to be analyzed or modified is large because these vectors are able to stably propagate large polynucleotides.

The vector containing the cloned DNA sequence can then be amplified by plating (i.e., clonal amplification) or transfecting a suitable host cell with the vector (e.g., a phage on an E. coli host). Alternatively (or subsequently to amplification), the cloned DNA sequence is used to prepare a library for screening by transforming a suitable organism. Hosts, known in the art are transformed by artificial introduction of the vectors containing the target nucleic acid by inoculation under conditions conducive for such transformation. One could transform with double stranded circular or linear nucleic acid or there may also be instances where one would transform with single stranded circular or linear nucleic acid sequences. By transform or transformation is meant a permanent or transient genetic change induced in a cell following incorporation of new DNA (i.e., DNA exogenous to the cell). Where the cell is a mammalian cell, a permanent genetic change is generally achieved by introduction of the DNA into the genome of the cell. A transformed cell or host cell generally refers to a cell (e.g., prokaryotic or eukaryotic) into which (or into an ancestor of which) has been introduced, by means of recombinant DNA techniques, a DNA molecule not normally present in the host organism.

A particularly type of vector for use in the invention contains an f-factor origin replication. The f-factor (or fertility factor) in E. coli is a plasmid which effects high frequency transfer of itself during conjugation and less frequent transfer of the bacterial chromosome itself. In a particular embodiment cloning vectors referred to as “fosmids” or bacterial artificial chromosome (BAC) vectors are used. These are derived from E. coli f-factor which is able to stably integrate large segments of DNA. When integrated with DNA from a mixed uncultured mixed population sample, this makes it possible to achieve large genomic fragments in the form of a stable “mixed population nucleic acid library.”

The nucleic acids derived from a mixed population or sample may be inserted into the vector by a variety of procedures. In general, the nucleic acid sequence is inserted into an appropriate restriction endonuclease site(s) by procedures known in the art. Such procedures and others are deemed to be within the scope of those skilled in the art. A typical cloning scenario may have the DNA “blunted” with an appropriate nuclease (e.g., Mung Bean Nuclease), methylated with, for example, EcoR I Methylase and ligated to EcoR I linkers. The linkers are then digested with an EcoR I Restriction Endonuclease and the DNA size fractionated (e.g., using a sucrose gradient). The resulting size fractionated DNA is then ligated into a suitable vector for sequencing, screening or expression (e.g., a lambda vector and packaged using an in vitro lambda packaging extract).

Transformation of a host cell with recombinant DNA may be carried out by conventional techniques as are well known to those skilled in the art. Where the host is prokaryotic, such as E. coli, competent cells which are capable of DNA uptake can be prepared from cells harvested after exponential growth phase and subsequently treated by the CaCl₂ method by procedures well known in the art. Alternatively, MgCl₂ or RbCl can be used. Transformation can also be performed after forming a protoplast of the host cell or by electroporation. Transformation of Pseudomonas fluorescens and yeast host cells can be achieved by electroporation, using techniques described herein.

When the host is a eukaryote, methods of transfection or transformation with DNA include conjugation, calcium phosphate co-precipitates, conventional mechanical procedures such as microinjection, electroporation, insertion of a plasmid encased in liposomes, or virus vectors, as well as others known in the art, may be used. Eukaryotic cells can also be cotransfected with a second foreign DNA molecule encoding a selectable marker, such as the herpes simplex thymidine kinase gene. Another method is to use a eukaryotic viral vector, such as simian virus 40 (SV40) or bovine papilloma virus, to transiently infect or transform eukaryotic cells and express the protein. (Eukaryotic Viral Vectors, Cold Spring Harbor Laboratory, Gluzman ed., 1982). The eukaryotic cell may be a yeast cell (e.g., Saccharomyces cerevisiae), an insect cell (e.g., Drosophila sp.) or may be a mammalian cell, including a human cell.

Eukaryotic systems, and mammalian expression systems, allow for post-translational modifications of expressed mammalian proteins to occur. Eukaryotic cells which possess the cellular machinery for processing of the primary transcript, glycosylation, phosphorylation, and, advantageously secretion of the gene product should be used. Such host cell lines may include, but are not limited to, CHO, VERO, BHK, HeLa, COS, MDCK, Jurkat, HEK-293, and W138.

After the gene libraries have been generated one can perform “biopanning” of the libraries prior to expression screening. The “biopanning” procedure refers to a process for identifying clones having a specified biological activity by screening for sequence homology in the library of clones, using at least one probe DNA comprising at least a portion of a DNA sequence encoding a polypeptide having the specified biological activity; and detecting interactions with the probe DNA to a substantially complementary sequence in a clone. Clones (either viable or non-viable) are then separated by an analyzer (e.g., a FACS apparatus or an apparatus that detects non-optical markers).

The probe DNA used to probe for the target DNA of interest contained in clones prepared from polynucleotides in a mixed population of organisms can be a full-length coding region sequence or a partial coding region sequence of DNA for a known bioactivity. The sequence of the probe can be generated by synthetic or recombinant means and can be based upon computer based sequencing programs or biological sequences present in a clone. The DNA library can be probed using mixtures of probes comprising at least a portion of the DNA sequence encoding a known bioactivity having a desired activity. These probes or probe libraries are preferably single-stranded. The probes that are particularly suitable are those derived from DNA encoding bioactivities having an activity similar or identical to the specified bioactivity which is to be screened.

In another embodiment, a nucleic acid library from a mixed population of organisms is screened for a sequence of interest by transfecting a host cell containing the library with at least one labeled nucleic acid sequence which is all or a portion of a DNA sequence encoding a bioactivity having a desirable activity and separating the library clones containing the desirable sequence by optical- or non-optical-based analysis.

In another embodiment, in vivo biopanning may be performed utilizing a FACS-based machine. Complex gene libraries are constructed with vectors which contain elements which stabilize transcribed RNA. For example, the inclusion of sequences which result in secondary structures such as hairpins which are designed to flank the transcribed regions of the RNA would serve to enhance their stability, thus increasing their half life within the cell. The probe molecules used in the biopanning process consist of oligonucleotides labeled with reporter molecules that only fluoresce upon binding of the probe to a target molecule. Various dyes or stains well known in the art, for example those described in “Practical Flow Cytometry”, 1995 Wiley-Liss, Inc., Howard M. Shapiro, M.D., can be used to intercalate or associate with nucleic acid in order to “label” the oligonucleotides. These probes are introduced into the recombinant cells of the library using one of several transformation methods. The probe molecules interact or hybridize to the transcribed target mRNA or DNA resulting in DNA/RNA heteroduplex molecules or DNA/DNA duplex molecules. Binding of the probe to a target will yield a fluorescent signal which is detected and sorted by the FACS machine during the screening process.

The probe DNA should be at least about 10 bases and preferably at least 15 bases. Desirable size ranges for probe DNA are at least about 15 bases to about 100 bases, at least about 100 bases to about 500 bases, at least about 500 bases to about 1,000 bases, at least about 1,000 bases to about 5,000 bases and at least about 5,000 bases to about 10,000 bases. In one embodiment, an entire coding region of one part of a pathway may be employed as a probe. Where the probe is hybridized to the target DNA in an in vitro system, conditions for the hybridization in which target DNA is selectively isolated by the use of at least one DNA probe will be designed to provide a hybridization stringency of at least about 50% sequence identity, more particularly a stringency providing for a sequence identity of at least about 70%. Hybridization techniques for probing a microbial DNA library to isolate target DNA of potential interest are well known in the art and any of those which are described in the literature are suitable for use herein. Prior to fluorescence sorting the clones may be viable or non-viable. For example, in one embodiment, the cells are fixed with paraformaldehyde prior to sorting.

Once viable or non-viable clones containing a sequence substantially complementary to the probe DNA are separated by a fluorescence analyzer, polynucleotides present in the separated clones may be further manipulated. In some instances, it may be desirable to perform an amplification of the target DNA that has been isolated. In this embodiment, the target DNA is separated from the probe DNA after isolation. In one embodiment, the clone can be grown to expand the clonal population. Alternatively, the host cell is lysed and the target DNA amplified. It is then amplified before being used to transform a new host (e.g., subcloning). Long PCR (Barnes, W M, Proc. Natl. Acad. Sci, USA, Mar. 15, 1994) can be used to amplify large DNA fragments (e.g., 35 kb). Numerous amplification methodologies are now well known in the art.

Where the target DNA is identified in vitro, the selected DNA is then used for preparing a library for further processing and screening by transforming a suitable organism. Hosts, particularly those specifically identified herein as preferred, are transformed by artificial introduction of a vector containing a target DNA by inoculation under conditions conducive for such transformation.

The resultant libraries (enriched for a polynucleotide of interest) can then be screened for clones which display an activity of interest. Clones can be shuttled in alternative hosts for expression of active compounds, or screened using methods described herein.

Having prepared a multiplicity of clones from DNA selectively isolated via hybridization technologies described herein, such clones are screened for a specific activity to identify clones having a specified characteristic.

The screening for activity may be effected on individual expression clones or may be initially effected on a mixture of expression clones to ascertain whether or not the mixture has one or more specified activities. If the mixture has a specified activity, then the individual clones may be rescreened for such activity or for a more specific activity.

Prior to, subsequent to or as an alternative to the in vivo biopanning described above is an encapsulation techniques such as GMDs, which may be employed to localize at least one clone in one location for growth or screening by a fluorescent analyzer (e.g. FACS). The separated at least one clone contained in the GMD may then be cultured to expand the number of clones or screened on a FACS machine to identify clones containing a sequence of interest as described above, which can then be broken out into individual clones to be screened again on a FACS machine to identify positive individual clones. Screening in this manner using a FACS machine is described in patent application Ser. No. 08/876,276, filed Jun. 16, 1997, herein incorporated by reference. Thus, for example, if a clone has a desirable activity, then the individual clones may be recovered and rescreened utilizing a FACS machine to determine which of such clones has the specified desirable activity.

Further, it is possible to combine some or all of the above embodiments such that a normalization step is performed prior to generation of the expression library, the expression library is then generated, the expression library so generated is then biopanned, and the biopanned expression library is then screened using a high throughput cell sorting and screening instrument. Thus there are a variety of options, including: (i) generating the library and then screening it; (ii) normalize the target DNA, generate the expression library and screen it; (iii) normalize, generate the library, biopan and screen; or (iv) generate, biopan and screen the library.

The library may, for example, be screened for a specified enzyme activity. For example, the enzyme activity screened for may be one or more of the six IUB classes; oxidoreductases, transferases, hydrolases, lyases, isomerases and ligases. The recombinant enzymes which are determined to be positive for one or more of the IUB classes may then be rescreened for a more specific enzyme activity.

Alternatively, the library may be screened for a more specialized enzyme activity. For example, instead of generically screening for hydrolase activity, the library may be screened for a more specialized activity, i.e. the type of bond on which the hydrolase acts. Thus, for example, the library may be screened to ascertain those hydrolases which act on one or more specified chemical functionalities, such as: (a) amide (peptide bonds), i.e. proteases; (b) ester bonds, i.e. esterases and lipases; (c) acetals, i.e., glycosidases etc.

As described with respect to one of the above aspects, the invention provides a process for activity screening of clones containing selected DNA derived from a mixed population of organisms or more than one organism.

Biopanning polynucleotides from a mixed population of organisms by separating the clones or polynucleotides positive for sequence of interest with a fluorescent analyzer that detects fluorescence, to select polynucleotides or clones containing polynucleotides positive for a sequence of interest, and screening the selected clones or polynucleotides for specified bioactivity. In one embodiment, the polynucleotides are contained in clones having been prepared by recovering DNA of a microorganism, which DNA is selected by hybridization to at least one DNA sequence which is all or a portion of a DNA sequence encoding a bioactivity having a desirable activity.

In another embodiment, a DNA library derived from a microorganism is subjected to a selection procedure to select therefrom DNA which hybridizes to one or more probe DNA sequences which is all or a portion of a DNA sequence encoding an activity having a desirable activity by:

(a) contacting a DNA library with a fluorescent labeled DNA probe under conditions permissive of hybridization so as to produce a double-stranded complex of probe and members of the DNA library.

The present invention offers the ability to screen for many types of bioactivities. For instance, the ability to select and combine desired components from a library of polyketides and postpolyketide biosynthesis genes for generation of novel polyketides for study is appealing. The method(s) of the present invention make it possible to and facilitate the cloning of novel polyketide synthase genes and/or gene pathways, and other relevant pathways or genes encoding commercially relevant secondary metabolites, since one can generate gene banks with clones containing large inserts (especially when using vectors which can accept large inserts, such as the f-factor based vectors), which facilitates cloning of gene clusters.

The biopanning approach described above can be used to create libraries enriched with clones carrying sequences substantially homologous to a given probe sequence. Using this approach libraries containing clones with inserts of up to 40 kbp or larger can be enriched approximately 1,000 fold after each round of panning. This enables one to reduce the number of clones to be screened after 1 round of biopanning enrichment. This approach can be applied to create libraries enriched for clones carrying sequence of interest related to a bioactivity of interest, for example, polyketide sequences.

Hybridization screening using high density filters or biopanning has proven an efficient approach to detect homologues of pathways containing genes of interest to discover novel bioactive molecules that may have no known counterparts. Once a polynucleotide of interest is enriched in a library of clones it may be desirable to screen for an activity. For example, it may be desirable to screen for the expression of small molecule ring structures or “backbones”. Because the genes encoding these polycyclic structures can often be expressed in E. coli, the small molecule backbone can be manufactured, even if in an inactive form. Bioactivity is conferred upon transferring the molecule or pathway to an appropriate host that expresses the requisite glycosylation and methylation genes that can modify or “decorate” the structure to its active form. Thus, even if inactive ring compounds, recombinantly expressed in E. coli are detected to identify clones which are then shuttled to a metabolically rich host, such as Streptomyces (e.g., Streptomyces diversae or venezuelae) for subsequent production of the bioactive molecule. It should be understood that E. coli can produce active small molecules and in certain instances it may be desirable to shuttle clones to a metabolically rich host for “decoration” of the structure, but not required. The use of high throughput robotic systems allows the screening of hundreds of thousands of clones in multiplexed arrays in microtiter dishes.

One approach to detect and enrich for clones carrying these structures is to use FACS screening, a procedure described and exemplified in U.S. Ser. No. 08/876,276, filed Jun. 16, 1997. Polycyclic ring compounds typically have characteristic fluorescent spectra when excited by ultraviolet light. Thus, clones expressing these structures can be distinguished from background using a sufficiently sensitive detection method. High throughput FACS screening can be utilized to screen for small molecule backbones in, for example, E. coli libraries. Commercially available FACS machines are capable of screening up to 100,000 clones per second for UV active molecules. These clones can be sorted for further FACS screening or the resident plasmids can be extracted and shuttled to Streptomyces for activity screening.

In another embodiment, a bioactivity or biomolecule or compound is detected by using various electromagnetic detection devices, including, for example, optical, magnetic and thermal detection associated with a flow cytometer.

Flow cytometer typically use an optical method of detection (fluorescence, scatter, and the like) to discriminate individual cells or particles from within a large population. There are several non-optical technologies that could be used alone or in conjunction with the optical methods to enable new discrimination/screening paradigms.

Magnetic field sensing is one such techniques that can be used as an alternative or in conjunction with, for example, fluorescence based methods. Hall-Effect Sensors are one example of sensors that can be employed. Superconducting Quantum Interference Devices (“SQUIDS”) are the most sensitive sensors for magnetic flux and magnetic fields, so far developed. A standardized criteria for the sensitivity of a SQUID is its energy resolution. This is defined as the smallest change in energy that the SQUID can detect in one second (or in a bandwidth of 1 Hz). Typical values are 10-33 J/Hz. The utility of SQUIDS can be found in the presence of magnetosomes in certain types of bacterial that contain chains of permanent single magnetic domain particles of magnetite (FE₃O₄) of gregite (Fe₃S₄). The magnetic field (or residual magnetic field) of a cell that contains a magnetosome is detected by positioning a SQUID in close proximity to the flow stream of a flow cytometer. Using this method cells or cells containing, for example, magnetic probes can be isolated based on their magnetic properties. As another example, changes in the synthetic pathway of magnetosome containing bacteria can be measured using a similar technique. Such techniques can be used to identify agents which modulate the synthetic pathway of magnetosomes.

Measuring dynamic charge properties is another techniques that can be used as an alternative or in conjunction with, for example, fluorescence based methods. Multipole Coupling Spectroscopy (“MCS”) directly measures the dynamic charge properties of systems without the need for labeling. Structural changes that occur when molecules interact result in representative changes in charge distribution, and these produce a dielectric based spectra or “signature” that reveals the affinity, specificity and functionality of each interaction. Similar changes in charge distribution occur in cellular systems. By observing the changes in these signatures, the dynamics of molecular pathways and cellular function can be resolved in their native conditions. MCS utilizes a small microwave (500 MHz to 50 GHz) transceiver that could be positioned in close proximity to the flow stream of a flow cytometer. Because of the short measurement times (e.g., microseconds) required, a complete MCS signature for each cell within the stream of a flow cytometer can be generated and analyzed. Certain cells can then be sorted and/or isolated based on either spectral features that are known a priori or based on some statistical variation from a general population. Examples of uses for this technique include selection of expression mutants, small molecule pre-screening, and the like.

In one screening approach, biomolecules from candidate clones can be tested for bioactivity by susceptibility screening against test organisms such as Staphylococcus aureus, Micrococcus luteus, E. coli, or Saccharomyces cervisiae. FACS screening can be used in this approach by co-encapsulating clones with the test organism.

An alternative to the above-mentioned screening methods provided by the present invention is an approach termed “mixed extract” screening. The “mixed extract” screening approach takes advantage of the fact that the accessory genes needed to confer activity upon the polycyclic backbones are expressed in metabolically rich hosts, such as Streptomyces, and that the enzymes can be extracted and combined with the backbones extracted from E. coli clones to produce the bioactive compound in vitro. Enzyme extract preparations from metabolically rich hosts, such as Streptomyces strains, at various growth stages are combined with pools of organic extracts from E. coli libraries and then evaluated for bioactivity. Another approach to detect activity in the E. coli clones is to screen for genes that can convert bioactive compounds to different forms. For example, a recombinant enzyme was recently discovered that can convert the low value daunomycin to the higher value doxorubicin. Similar enzyme pathways are being sought to convert penicillins to cephalosporins.

Screening may be carried out to detect a specified enzyme activity by procedures known in the art. For example, enzyme activity may be screened for one or more of the six IUB classes; oxidoreductases, transferases, hydrolases, lyases, isomerases and ligases. The recombinant enzymes which are determined to be positive for one or more of the IUB classes may then be rescreened for a more specific enzyme activity. Alternatively, the library may be screened for a more specialized enzyme activity. For example, instead of generically screening for hydrolase activity, the library may be screened for a more specialized activity, i.e. the type of bond on which the hydrolase acts. Thus, for example, the library may be screened to ascertain those hydrolases which act on one or more specified chemical functionalities, such as: (a) amide (peptide bonds), i.e. proteases; (b) ester bonds, i.e. esterases and lipases; (c) acetals, i.e., glycosidases.

FACS screening can also be used to detect expression of UV fluorescent molecules in any host, including metabolically rich hosts, such as Streptomyces. For example, recombinant oxytetracylin retains its diagnostic red fluorescence when produced heterologously in S. lividans TK24. Pathway clones, which can be sorted by FACS, can thus be screened for polycyclic molecules in a high throughput fashion.

Recombinant bioactive compounds can also be screened in vivo using “two-hybrid” systems, which can detect enhancers and inhibitors of protein-protein or other interactions such as those between transcription factors and their activators, or receptors and their cognate targets. In this embodiment, both the small molecule pathway and the reporter construct are co-expressed. Clones altered in reporter expression can then be sorted by FACS and the pathway clone isolated for characterization.

As indicated, common approaches to drug discovery involve screening assays in which disease targets (macromolecules implicated in causing a disease) are exposed to potential drug candidates which are tested for therapeutic activity. In other approaches, whole cells or organisms that are representative of the causative agent of the disease, such as bacteria or tumor cell lines, are exposed to the potential candidates for screening purposes. Any of these approaches can be employed with the present invention.

The present invention also allows for the transfer of cloned pathways derived from uncultivated samples into metabolically rich hosts for heterologous expression and downstream screening for bioactive compounds of interest using a variety of screening approaches briefly described above.

Recovering Desirable Bioactivities

After viable or non-viable cells, each containing a different expression clone from the gene library are screened, and positive clones are recovered, DNA can be isolated from positive clones utilizing techniques well known in the art. The DNA can then be amplified either in vivo or in vitro by utilizing any of the various amplification techniques known in the art. In vivo amplification would include transformation of the clone(s) or subclone(s) into a viable host, followed by growth of the host. In vitro amplification can be performed using techniques such as the polymerase chain reaction. Once amplified the identified sequences can be “evolved” or sequenced.

Evolution

One advantage afforded by present invention is the ability to manipulate the identified polynucleotides to generate and select for encoded variants with altered activity or specificity.

Clones found to have the bioactivity for which the screen was performed can be subjected to directed mutagenesis to develop new bioactivities with desired properties or to develop modified bioactivities with particularly desired properties that are absent or less pronounced in the wild-type activity, such as stability to heat or organic solvents. Any of the known techniques for directed mutagenesis are applicable to the invention. For example, particularly preferred mutagenesis techniques for use in accordance with the invention include those described below.

Alternatively, it may be desirable to variegate a polynucleotide sequence obtained, identified or cloned as described herein. Such variegation can modify the polynucleotide sequence in order to modify (e.g., increase or decrease) the encoded polypeptide's activity, specificity, affinity, function, etc. Such evolution methods are known in the art or described herein, such as, shuffling, cassette mutagenesis, recursive ensemble mutagenesis, sexual PCR, directed evolution, exonuclease-mediated reassembly, codon site-saturation mutagenesis, amino acid site-saturation mutagenesis, gene site saturation mutagenesis, introduction of mutations by non-stochastic polynucleotide reassembly methods, synthetic ligation polynucleotide reassembly, gene reassembly, oligonucleotide-directed saturation mutagenesis, in vivo reassortment of polynucleotide sequences having partial homology, naturally occurring recombination processes which reduce sequence complexity, and any combination thereof.

The clones enriched for a desired polynucleotide sequence, which are identified as described above, may be sequenced to identify the DNA sequence(s) present in the clone, which sequence information can be used to screen a database for similar sequences or functional characteristics. Thus, in accordance with the present invention it is possible to isolate and identify: (i) DNA having a sequence of interest (e.g., a sequence encoding an enzyme having a specified enzyme activity), (ii) associate the sequence with known or unknown sequence in a database (e.g., database sequence associated with an enzyme having an activity (including the amino acid sequence thereof)), and (iii) produce recombinant enzymes having such activity.

Sequencing may be performed by high through-put sequencing techniques. The exact method of sequencing is not a limiting factor of the invention. Any method useful in identifying the sequence of a particular cloned DNA sequence can be used. In general, sequencing is an adaptation of the natural process of DNA replication. Therefore, a template (e.g., the vector) and primer sequences are used. One general template preparation and sequencing protocol begins with automated picking of bacterial colonies, each of which contains a separate DNA clone which will function as a template for the sequencing reaction. The selected clones are placed into media, and grown overnight. The DNA templates are then purified from the cells and suspended in water. After DNA quantification, high-throughput sequencing is performed using a sequencers, such as Applied Biosystems, Inc., Prism 377 DNA Sequencers. The resulting sequence data can then be used in additional methods, including to search a database or databases.

Database Searches and Alignment Algorithms

A number of source databases are available that contain either a nucleic acid sequence and/or a deduced amino acid sequence for use with the invention in identifying or determining the activity encoded by a particular polynucleotide sequence. All or a representative portion of the sequences (e.g., about 100 individual clones) to be tested are used to search a sequence database (e.g., GenBank, PFAM or ProDom), either simultaneously or individually. A number of different methods of performing such sequence searches are known in the art. The databases can be specific for a particular organism or a collection of organisms. For example, there are databases for the C. elegans, Arabadopsis. sp., M. genitalium, M. jannaschii, E. coli, H. influenzae, S. cerevisiae and others. The sequence data of the clone is then aligned to the sequences in the database or databases using algorithms designed to measure homology between two or more sequences.

Such sequence alignment methods include, for example, BLAST (Altschul et al., 1990), BLITZ (MPsrch) (Sturrock & Collins, 1993), and FASTA (Person & Lipman, 1988). The probe sequence (e.g., the sequence data from the clone) can be any length, and will be recognized as homologous based upon a threshold homology value. The threshold value may be predetermined, although this is not required. The threshold value can be based upon the particular polynucleotide length. To align sequences a number of different procedures can be used. Typically, Smith-Waterman or Needleman-Wunsch algorithms are used. However, as discussed faster procedures such as BLAST, FASTA, PSI-BLAST can be used.

For example, optimal alignment of sequences for aligning a comparison window may be conducted by the local homology algorithm of Smith (Smith and Waterman, Adv Appl Math, 1981; Smith and Waterman, J Teor Biol, 1981; Smith and Waterman, J Mol Biol, 1981; Smith et al, J Mol Evol, 1981), by the homology alignment algorithm of Needleman (Needleman and Wuncsch, 1970), by the search of similarity method of Pearson (Pearson and Lipman, 1988), by computerized implementations of these algorithms (GAP, BESTFIT, FASTA, and TFASTA in the Wisconsin Genetics Software Package Release 7.0, Genetics Computer Group, 575 Science Dr., Madison, Wis., or the Sequence Analysis Software Package of the Genetics Computer Group, University of Wisconsin, Madison, Wis.), or by inspection, and the best alignment (i.e., resulting in the highest percentage of homology over the comparison window) generated by the various methods is selected. The similarity of the two sequence (i.e., the probe sequence and the database sequence) can then be predicted.

Such software matches similar sequences by assigning degrees of homology to various deletions, substitutions and other modifications. The terms “homology” and “identity” in the context of two or more nucleic acids or polypeptide sequences, refer to two or more sequences or subsequences that are the same or have a specified percentage of amino acid residues or nucleotides that are the same when compared and aligned for maximum correspondence over a comparison window or designated region as measured using any number of sequence comparison algorithms or by manual alignment and visual inspection.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned.

One example of a useful algorithm is BLAST and BLAST 2.0 algorithms, which are described in Altschul et al., Nuc. Acids Res. 25:3389-3402 (1977) and Altschul et al., J. Mol. Biol. 215:403410 (1990), respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0). The BLAST algorithm parameters W, T, and X determine the sensitivity and speed of the alignment. The BLASTN program (for nucleotide sequences) uses as defaults a wordlength (W) of 11, an expectation (E) of 10, M=5, N=4 and a comparison of both strands.

The BLAST algorithm also performs a statistical analysis of the similarity between two sequences (see, e.g., Karlin & Altschul, Proc. Natl. Acad. Sci. USA 90:5873 (1993)). One measure of similarity provided by BLAST algorithm is the smallest sum probability (P(N)), which provides an indication of the probability by which a match between two nucleotide sequences would occur by chance. For example, a nucleic acid is considered similar to a references sequence if the smallest sum probability in a comparison of the test nucleic acid to the reference nucleic acid is less than about 0.2, more preferably less than about 0.01, and most preferably less than about 0.001.

Sequence homology means that two polynucleotide sequences are homolgous (i.e., on a nucleotide-by-nucleotide basis) over the window of comparison. A percentage of sequence identity or homology is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence homology. This substantial homology denotes a characteristic of a polynucleotide sequence, wherein the polynucleotide comprises a sequence having at least 60 percent sequence homology, typically at least 70 percent homology, often 80 to 90 percent sequence homology, and most commonly at least 99 percent sequence homology as compared to a reference sequence of a comparison window of at least 25-50 nucleotides, wherein the percentage of sequence homology is calculated by comparing the reference sequence to the polynucleotide sequence which may include deletions or additions which total 20 percent or less of the reference sequence over the window of comparison.

Sequences having sufficient homology can the be further identified by any annotations contained in the database, including, for example, species and activity information. Accordingly, in a typical mixed population sample, a plurality of nucleic acid sequences will be obtained, cloned, sequenced and corresponding homologous sequences from a database identified. This information provides a profile of the polynucleotides present in the sample, including one or more features associated with the polynucleotide including the organism and activity associated with that sequence or any polypeptide encoded by that sequence based on the database information. As used herein “fingerprint” or “profile” refers to the fact that each sample will have associated with it a set of polynucleotides characteristic of the sample and the environment from which it was derived. Such a profile can include the amount and type of sequences present in the sample, as well as information regarding the potential activities encoded by the polynucleotides and the organisms from which polynucleotides were derived. This unique pattern is each sample's profile or fingerprint.

In some instances it may be desirable to express a particular cloned polynucleotide sequence once its identity or activity is determined or an suggested identity or activity is associated with the polynucleotide. In such instances the desired clone, if not already cloned into an expression vector, is ligated downstream of a regulatory control element (e.g., a promoter or enhancer) and cloned into a suitable host cell. Expression vectors are commercially available along with corresponding host cells for use in the invention.

As representative examples of expression vectors which may be used there may be mentioned viral particles, baculovirus, phage, plasmids, phagemids, cosmids, fosmids, bacterial artificial chromosomes, viral nucleic acid (e.g., vaccinia, adenovirus, foul pox virus, pseudorabies and derivatives of SV40), P1-based artificial chromosomes, yeast plasmids, yeast artificial chromosomes, and any other vectors specific for specific hosts of interest (such as bacillus, aspergillus, yeast, etc.) Thus, for example, the DNA may be included in any one of a variety of expression vectors for expressing a polypeptide. Such vectors include chromosomal, nonchromosomal and synthetic DNA sequences. Large numbers of suitable vectors are known to those of skill in the art, and are commercially available. The following vectors are provided by way of example; ZAP Express, Lambda ZAP®-CMV, Lambda ZAP® II, Lambda gt10, Lambda gtl 11, pMyr, pSos, pCMV-Script, pCMV-Script XR, pBK Phagemid, pBK-CMV, pBK-RSV, pBluescript II Phagemid, pBluescript II KS +, pBluescript II SK +, pBluescript II SK −, Lambda FIX II, Lambda DASH II, Lambda EMBL3 and EMBL4, EMBL3, EMBL4, SuperCos I and pWE15, pWE15, SuperCos I, pPCR-Script Amp, pPCR-Script Cam, pCMV-Script, pBC KS +, pBC KS −, pBC SK +, pBC SK −, psiX174, pNH8A, pNH16a, pNH18A, pNH46A (Stratagene); PT7BLUE, pSTBlue, pCITE, pET, ptriEx, pForce (Novagen); pIND-E, pIND Vector, pIND/Hygro, pIND(SP1)/Hygro, pIND/GFP, pIND(SP1)/GFP, pIND/V5-His and pIND(SP1)/V5-His Tag, pIND TOPO TA, pShooter™ Targeting Vectors, pTracer™ GFP Reporter Vectors, pcDNA® Vector Collection, EBV Vectors, Voyager™ VP22 Vectors, pVAX1-DNA vaccine vector, pcDNA4/His-Max, pBC1 Mouse Milk System (Invitrogen); pQE70, pQE60, pQE-9, pQE-16, pQE-30/pQE-80, pQE 31/pQE 81, pQE-32/pQE 82, pQE-40, pQE-100 Double Tag (Qiagen); pTRC99a, pKK223-3, pKK233-3, pDR540, pRIT5, pWLNEO, pSV2CAT, pOG44, pXT1, pSG (Stratagene), pSVK3, pBPV, pMSG, pSVL (Pharmacia).However, any other plasmid or vector may be used as long as they are replicable and viable in the host.

The nucleic acid sequence in the expression vector is operatively linked to an appropriate expression control sequence(s) (promoter) to direct mRNA synthesis. Particular named bacterial promoters include lacI, lacZ, T3, T7, gpt, lambda PR, PL, SP6, trp, lacUV5, PBAD, araBAD, araB, trc, proU, p-D-HSP, HSP, GAL4 UAS/E1b, TK, GAL1, CMV/TetO₂ Hybrid, EF-1a CMV, EF-1a CMV, EF-1a CMV, EF, EF-1a, ubiquitin C, rsv-ltr, rsv, b-lactamase, nmtl, and gal10. Eukaryotic promoters include CMV immediate early, HSV thymidine kinase, early and late SV40, LTRs from retrovirus, and mouse metallothionein-I. Selection of the appropriate vector and promoter is well within the level of ordinary skill in the art. The expression vector also contains a ribosome binding site for translation initiation and a transcription terminator. The vector may also include appropriate sequences for amplifying expression. Promoter regions can be selected from any desired gene using CAT (chloramphenicol transferase) vectors or other vectors with selectable markers.

In addition, the expression vectors preferably contain one or more selectable marker genes to provide a phenotypic trait for selection of transformed host cells such as dihydrofolate reductase or neomycin resistance for eukaryotic cell culture, or such as tetracycline or ampicillin resistance in E. coli.

The nucleic acid sequence(s) selected, cloned and sequenced as hereinabove described can additionally be introduced into a suitable host to prepare a library which is screened for the desired enzyme activity. The selected nucleic acid is preferably already in a vector which includes appropriate control sequences whereby a selected nucleic acid encoding an enzyme may be expressed, for detection of the desired activity. The host cell can be a higher eukaryotic cell, such as a mammalian cell, or a lower eukaryotic cell, such as a yeast cell, or the host cell can be a prokaryotic cell, such as a bacterial cell. The selection of an appropriate host is deemed to be within the scope of those skilled in the art from the teachings herein.

In some instances it may be desirable to perform an amplification of the nucleic acid sequence present in a sample or a particular clone that has been isolated. In this embodiment the nucleic acid sequence is amplified by PCR reaction or similar reaction known to those of skill in the art. Commercially available amplification kits are available to carry out such amplification reactions.

In addition, it is important to recognize that the alignment algorithms and searchable database can be implemented in computer hardware, software or a combination thereof. Accordingly, the isolation, processing and identification of nucleic acid sequences and the corresponding polypeptides encoded by those sequence can be implemented in and automated system.

Capillary-Based Screening

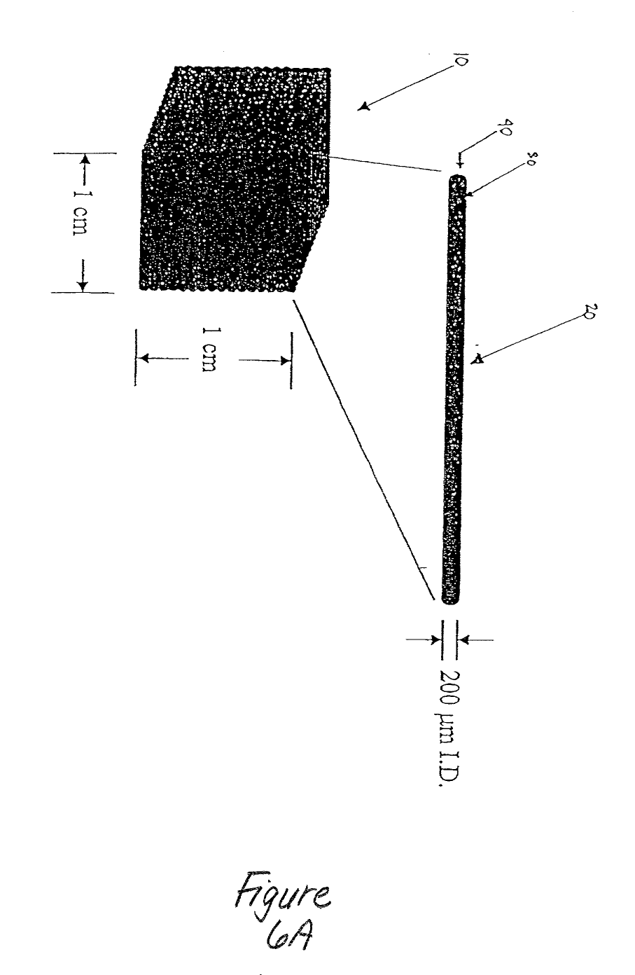

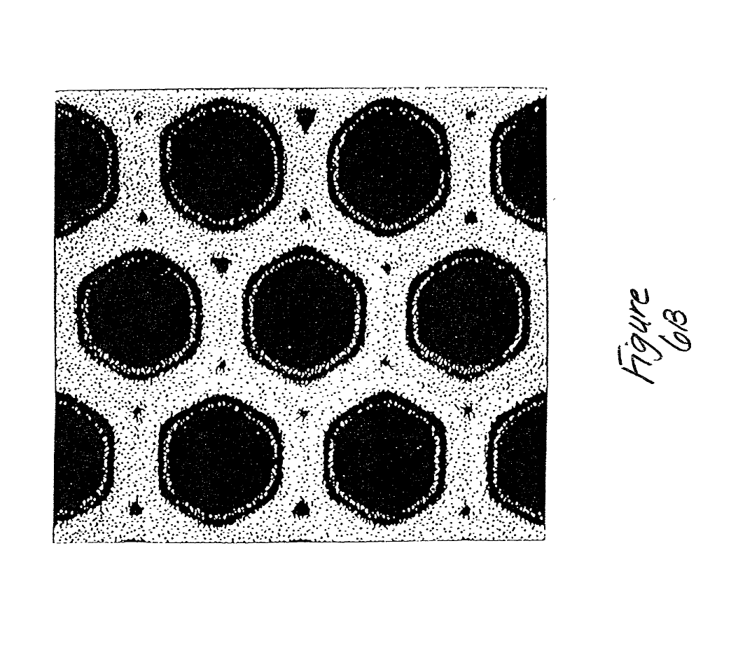

FIG. 6A shows a capillary array (10) which includes a plurality of individual capillaries (20) having at least one outer wall (30) defining a lumen (40). The outer wall (30) of the capillary (20) can be one or more walls fused together. Similarly, the wall can define a lumen (40) that is cylindrical, square, hexagonal or any other geometric shape so long as the walls form a lumen for retention of a liquid or sample. The capillaries (20) of the capillary array (10) are held together in close proximity to form a planar structure. The capillaries (20) can be bound together, by being fused (e.g., where the capillaries are made of glass), glued, bonded, or clamped side-by-side. The capillary array (10) can be formed of any number of individual capillaries (20). In an embodiment, the capillary array includes 100 to 4,000,000 capillaries (20). In one embodiment, the capillary array includes 100 to 500,000,000 capillaries (20). In one embodiment, the capillary array includes 100,000 capillaries (20). In one specific embodiment, the capillary array (10) can be formed to conform to a microtiter plate footprint, i.e. 127.76 mm by 85.47 mm, with tolerances. The capillary array (10) can have a density of 500 to more than 1,000 capillaries (20) per cm2, or about 5 capillaries per mm2. For example, a microtiter plate size array of 3 um capillaries would have about 500 million capillaries.

The capillaries (20) are preferably formed with an aspect ratio of 50:1. In one embodiment, each capillary (20) has a length of approximately 10 mm, and an internal diameter of the lumen (40) of approximately 200 μm. However, other aspect ratios are possible, and range from 10:1 to well over 1000:1. Accordingly, the thickness of the capillary array can vary from 0.5 mm to over 10 cm. Individual capillaries (20) have an inner diameter that ranges from 3-500 μm and 0-500 μm. A capillary (20) having an internal diameter of 200 μm and a length of 1 cm has a volume of approximately 0.3 μl. The length and width of each capillary (20) is based on a desired volume and other characteristics discussed in more detail below, such as evaporation rate of liquid from within the capillary, and the like. Capillaries of the invention may include a volume as low as 250 nanoliters/well.

In accordance with one embodiment of the invention, one or more particles are introduced into each capillary (20) for screening. Suitable particles include cells, cell clones, and other biological matter, chemical beads, or any other particulate matter. The capillaries (20) containing particles of interest can be introduced with various types of substances for causing an activity of interest. The introduced substance can include a liquid having a developer or nutrients, for example, which assists in cell growth and which results in the production of enzymes. Or, a chemical solution containing new particles can cause a combining event with other chemical beads already introduced into one or more capillaries (20). The particles and resulting activity of interest are screened and analyzed using the capillary array (10) according to the present invention. In one embodiment, the activity produces a change in properties of matter within the capillary (20), such as optical properties of the particles. Each capillary can act as a waveguide for guiding detectable light energy or property changes to an analyzer.

The capillaries (20) can be made according to various manufacturing techniques. In one particular embodiment, the capillaries (20) are manufactured using a hollow-drawn technique. A cylindrical, or other hollow shape, piece of glass is drawn out to continually longer lengths according to known techniques. The piece of glass is preferably formed of multiple layers. The drawn glass is then cut into portions of a specific length to form a relatively large capillary. The capillary portions are next bundled into an array of relatively large capillaries, and then drawn again to increasingly narrower diameters. During the drawing process, or when the capillaries are formed to a desired width, application of heat can fuse interstitial areas of adjacent capillaries together.

In an alternative embodiment, a glass etching process is used. Preferably, a solid tube of glass is drawn out to a particular width, cut into portions of a specific length, and drawn again. Then, each solid tube portion is center-etched with an acid or other etchant to form a hollow capillary. The tubes can be bound or fused together before or after the etch process.

A number of capillary arrays (10) can be connected together to form an array of arrays (12), as shown in FIG. 6B. The capillary arrays (10) can be glued together. Alternatively, the capillary arrays (10) can be fused together. According to this technique, the array of arrays (12) can have any desired size or footprint, formed of any number of high-precision capillary arrays (10).

A large number of materials can be suitably used to form a capillary array according to the invention and depending on the manufacturing technique used, including without limitation, glass, metal, semiconductors such as silicon, quartz, ceramics, or various polymers and plastics including, among others, polyethylene, polystyrene, and polypropylene. The internal walls of the capillary array, or portions thereof, may be coated or silanized to modify their surface properties. For example, the hydrophilicity or hydrophobicity may be altered to promote or reduce wicking or capillary action, respectively. The coating material includes, for example, ligands such as avidin, streptavidin, antibodies, antigens, and other molecules having specific binding affinity or which can withstand thermal or chemical sterilization.

While the above-described manufacturing techniques and materials yield high precision micro-sized capillaries and capillary arrays, the size, spacing and alignment of the capillaries within an array may be non-uniform. In some instances, it is desirable to have two capillary arrays make contact in as close alignment as possible, such as, for example, to transfer liquid from capillaries in a first capillary array to capillaries in a second capillary array. One capillary array according to the invention may be cut horizontally along its thickness, and separated to form two capillary arrays. The two resulting capillary arrays will each include at least one surface having capillary openings of substantially identical size, spacing and alignment, and suitable for contacting together for transferring liquid from one resulting capillary array to the other.

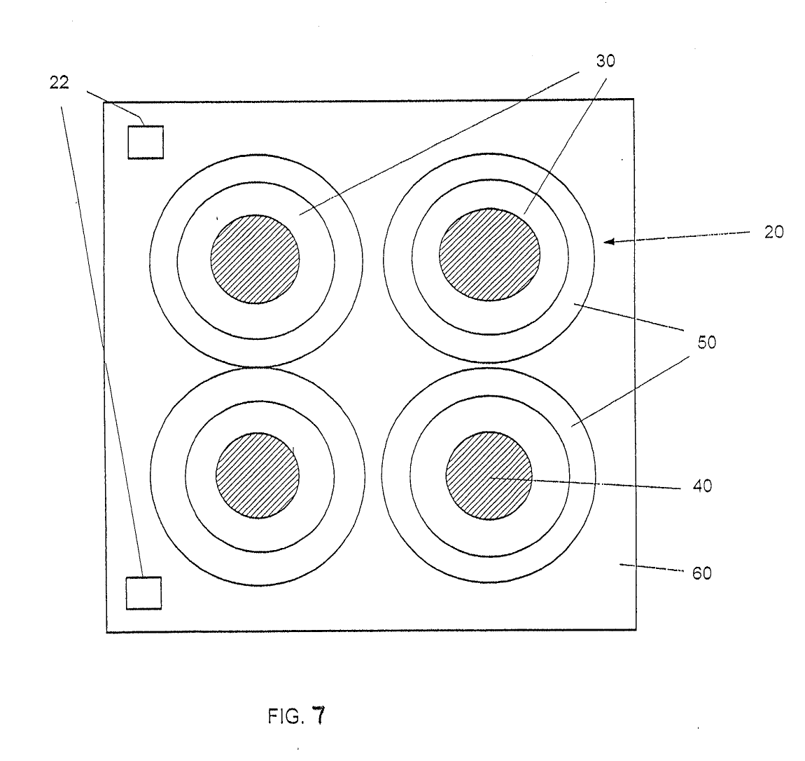

FIG. 7 shows a horizontal cross section of a portion of an array of capillaries (20). Capillary (20) is shown having a first cylindrical wall (30), a lumen (40), a second exterior wall (50), and interstitial material (60) separating the capillary tubes in the array (10). In this embodiment, the cylindrical wall (30) is comprised of a sleeve glass, while exterior wall (50) is comprised of an extra mural absorption (EMA) glass to minimize optical crosstalk among neighboring capillaries (20).

A capillary array may optionally include reference indicia (22) for providing a positional or alignment reference. The reference indicia (22) may be formed of a pad of glass extending from the surface of the capillary array, or embedded in the interstitial material (60). In one embodiment, the reference indicia (22) are provided at one or more corners of a microtiter plate formed by the capillary array. According to the embodiment, a corner of the plate or set of capillaries may be removed, and replaced with the reference indicia (22). The reference indicia (22) may also be formed at spaced intervals along a capillary array, to provide an indication of a subset of capillaries (20).

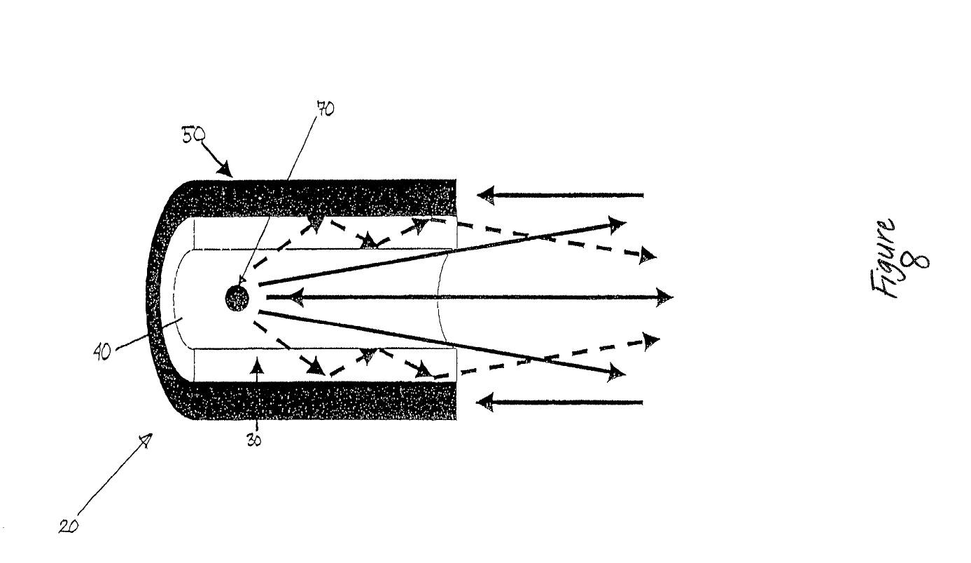

FIG. 8 depicts a vertical cross-section of a capillary of the invention. The capillary (20) includes a first wall (30) defining a lumen (40), and a second wall (50) surrounding the first wall (30). In one embodiment, the second wall (50) has a lower index of refraction than the first wall (30). In one embodiment, the first wall (30) is sleeve glass having a high index of refraction, forming a waveguide in which light from excited fluorophores travels. In the exemplary embodiment, the second wall (50) is black EMA glass, having a low index of refraction, forming a cladding around the first wall (30) against which light is refracted and directed along the first wall (30) for total internal reflection within the capillary (20). The second wall (50) can thus be made with any material that reduces the “cross-talk” or diffusion of light between adjacent capillaries. Alternatively, the inside surface of the first wall (30) can be coated with a reflective substance to form a mirror, or mirror-like structure, for specular reflection within the lumen (40).

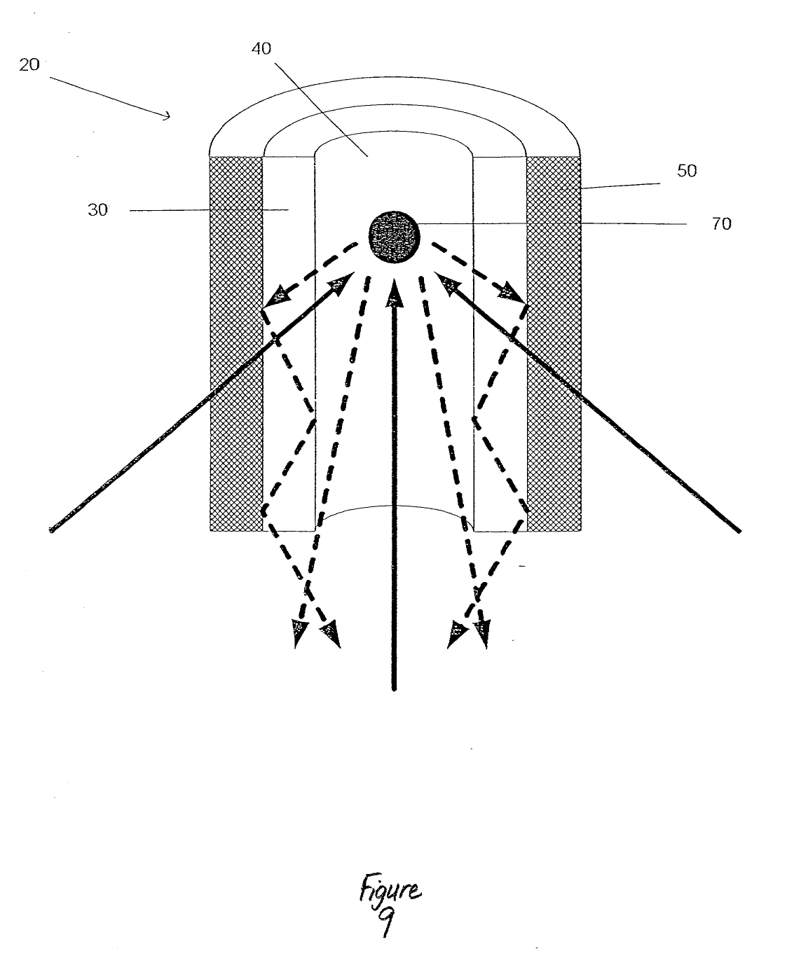

Many different materials can be used in forming the first and second walls, creating different indices of refraction for desired purposes. A filtering material can be formed around the lumen (40) to filter energy to and from the lumen (40) as depicted in FIG. 9. In one embodiment, the inner wall of the first wall (30) of each capillary of the array, or portion of the array, is coated with the filtering material. In another embodiment, the second wall (50) includes the filtering material. For instance, the second wall (50) can be formed of the filtering material, such as filter glass for example, or in one exemplary embodiment, the second wall (50) is EMA glass that is doped with an appropriate amount of filtering material. The filtering material can be formed of a color other than black and tuned for a desired excitation/emission filtering characteristic.

The filtering material allows transmission of excitation energy into the lumen (40), and blocks emission energy from the lumen (40) except through one or more openings at either end of the capillary (20). In FIG. 9, excitation energy is illustrated as a solid line, while emission energy is indicated by a broken line. When the second wall (50) is formed with a filtering material as shown in FIG. 9, certain wavelengths of light representing excitation energy are allowed through to the lumen (40), and other wavelengths of light representing emission energy are blocked from exiting, except as directed within and along the first wall (30). The entire capillary array, or a portion thereof, can be tuned to a specific individual wavelength or group of wavelengths, for filtering different bands of light in an excitation and detection process.

A particle (70) is depicted within the lumen (40). During use, an excitation light is directed into the lumen (40) contacting the particle (70) and exciting a reporter fluorescent material causing emission of light. The emitted light travels the length of the capillary until it reaches a detector. One advantage of an embodiment of the present invention, where the second wall (50) is black EMA glass, is that the emitted light cannot cross contaminate adjacent capillary tubes in a capillary array. In addition, the black EMA glass refracts and directs the emitted light towards either end of the capillary tube thus increasing the signal detected by an optical detector (e.g., a CCD camera and the like).

In a detection process using a capillary array of the invention, an optical detection system is aligned with the array, which is then scanned for one or more bright spots, representing either a fluorescence or luminescence associated with a “positive.” The term “positive” refers to the presence of an activity of interest. Again, the activity can be a chemical event, or a biological event.

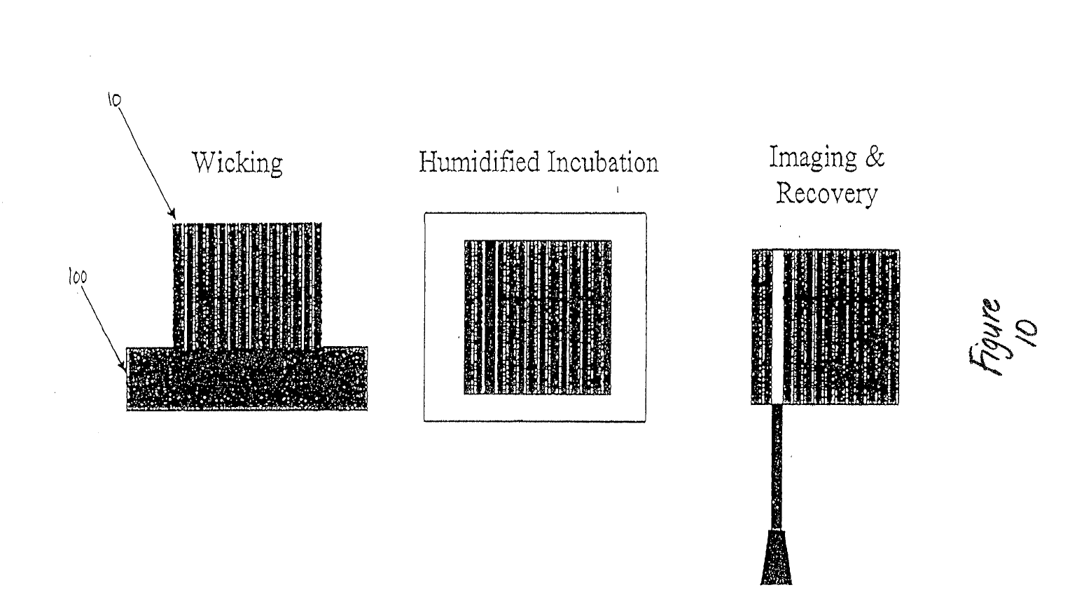

FIG. 10 depicts a general method of sample screening using a capillary array (10) according to the invention. In this depiction, capillary array (10) is immersed or contacted with a container (100) containing particles of interest. The particles can be cells, clones, molecules or compounds suspended in a liquid. The liquid is wicked into the capillary tubes by capillary action. The natural wicking that occurs as a result of capillary forces obviates the need for pumping equipment and liquid dispensers. A substrate for measuring biological activity (e.g., enzyme activity) can be contacted with the particles either before or after introduction of the particles into the capillaries in the capillary array. The substrate can include clones of a cell of interest, for example. The substrate can be introduced simultaneously into the capillaries by placing an open end of the capillaries in the container (100) containing a mixture of the particle-bearing liquid and the substrate. In some embodiments, it is a goal to achieve a certain concentration of particles of interest. A particular concentration of particles may also be achieved by dilution. FIGS. 13A-C show one such process, which is described below. Alternatively, the particle-bearing liquid may be wicked a portion of the way into the capillaries, and then the substrate is wicked into a remaining portion of the capillaries.

The mixture in the capillaries can then be incubated for producing a desired activity. The incubation can be for a specific period of time and at an appropriate temperature necessary for cell growth, for example, or to allow the substrate to permeabilize the cell membrane to produce an optically detectable signal, or for a period of time and at a temperature for optimum enzymatic activity. The incubation can be performed, for example, by placing the capillary array in a humidified incubator or in an apparatus containing a water source to ensure reduced evaporation within the capillary tubes. Evaporative loss may be reduced by increasing the relative humidity (e.g., by placing the capillary array in a humidified chamber). The evaporation rate can also be reduced by capping the capillaries with an oil, wax, membrane or the like. Alternatively, a high molecular weight fluid such as various alcohols, or molecules capable of forming a molecular monolayer, bilayers or other thin films (e.g., fatty acids), or various oils (e.g., mineral oil) can be used to reduce evaporation.

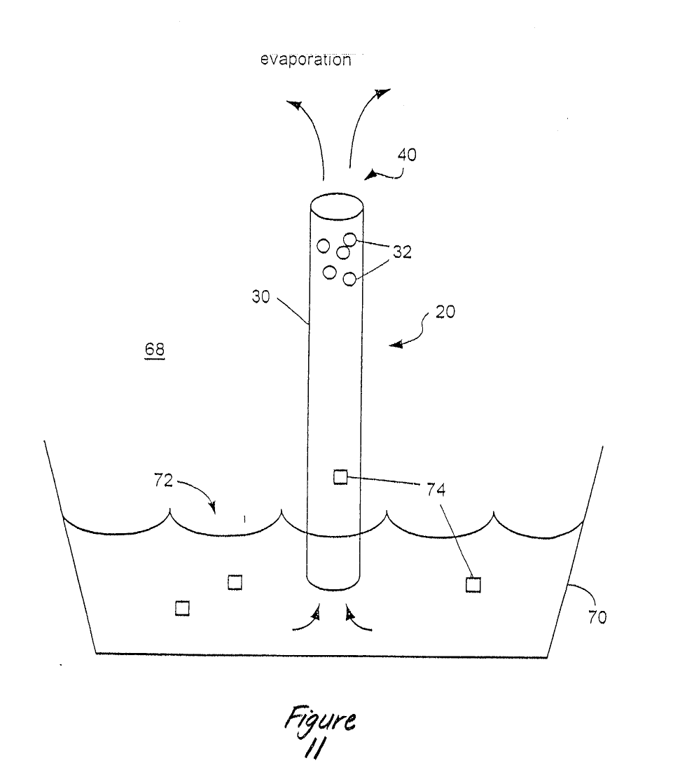

FIG. 11 illustrate a method for incubating a substrate solution containing cells of interest. While only a single capillary (20) is shown in FIG. 11 for simplicity, it should be understood that the incubation method applies to a capillary array having a plurality of capillaries (20). In accordance with one embodiment, a first fluid is wicked into the capillary (20) according to methods described above. The capillary (20) containing the substrate solution and cells (32) is then introduced to a fluid bath (70) containing a second liquid (72). The second liquid may or may not be the same as the first. For instance, the first liquid may contain particles (32) from which an activity is screened. The particles (32) are suspended in liquid within the lumen (40), and gradually migrate toward the top of the lumen (40) in the direction of the flow of liquid through the capillary (20) due to evaporation. The width of the lumen (40) at the open end of the capillary (20) is sized to provide a particular surface area of liquid at the top of the lumen (40), for controlling the amount and rate of evaporation of the liquid mixture. By controlling the environment (68) near the non-submersed end of the capillary (20), the first liquid from within the capillary (20) will evaporate, and will be replenished by the second liquid (72) from the fluid bath (70).

The amount of evaporation is balanced against possible diffusion of the contents of the capillary (20) into the liquid (72), and against possible mechanical mixing of the capillary contents with the liquid (72) due to vibration and pressure changes. The greater the width of the lumen (40), the larger the amount of mechanical mixing. Therefore, the temperature and humidity level in the surrounding environment may be adjusted to produce the desired evaporative cycle, and the lumen (40) width is sized to minimize mechanical mixing, in addition to produce a desired evaporation rate. The non-submersed open end of the capillary (20) may also be capped to create a vacuum force for holding the capillary contents within the capillary, and minimizing mechanical mixing and diffusion of the contents within the liquid (72). However when capped, the capillary (20) will not experience evaporation.

The liquid (72) can be supplemented with nutrients (74) to support a greater likelihood or rate of activity of the particles (32). For example, oxygen can be added to the liquid to nourish cells or to optimize the incubation environment of the cells. In another example, the liquid (72) can contain a substrate or a recombinant clone, or a developer for the particles (32). The cells can be optimally cultured by controlling the amount and rate of evaporation. For instance, by decreasing relative humidity of the environment (68), evaporation from the lumen (40) is increased, thereby increasing a rate of flow of liquid (72) through the capillary (20). Another advantage of this method is the ability to control conditions within the capillary (20) and the environment (68) that are not otherwise possible.

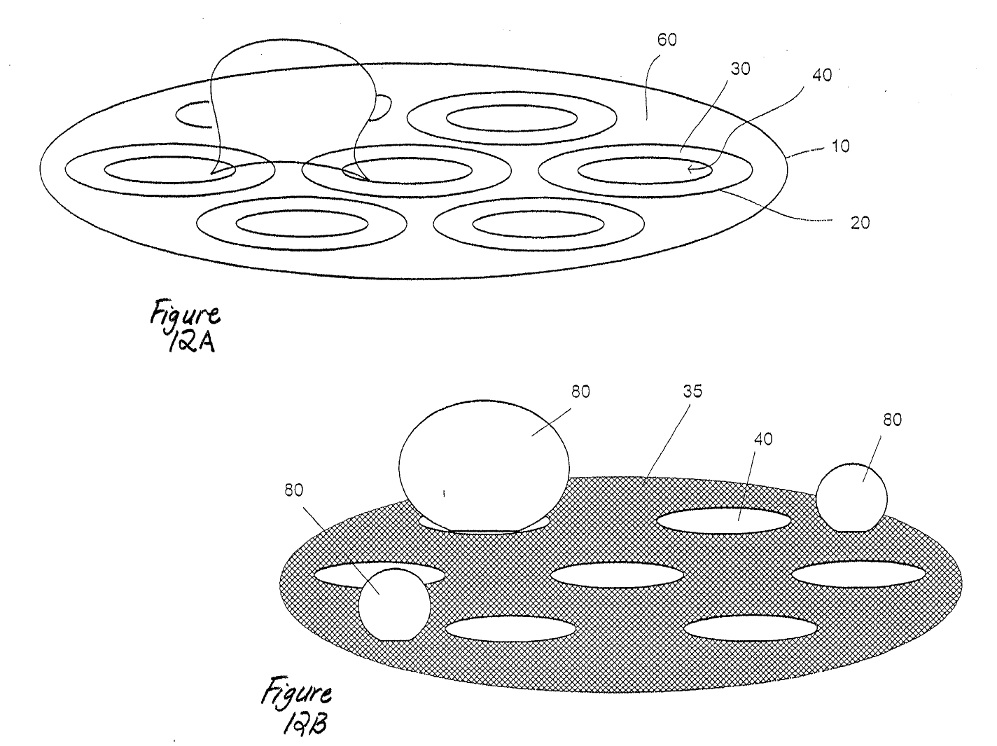

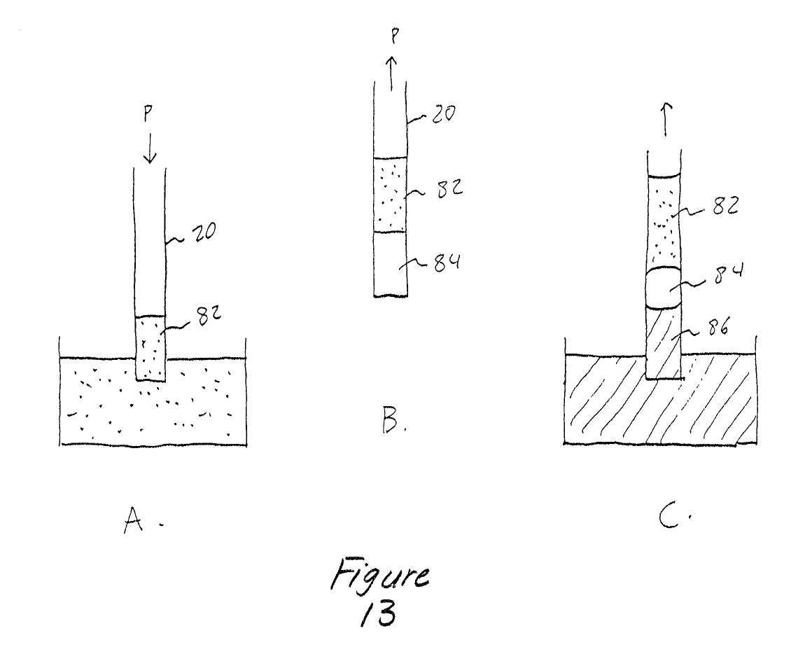

A relatively high humidity level of the environment will slow the rate of evaporation and keep more liquid within the capillary (20). If a temperature differential exists between a capillary array (10) and its environment, however, condensation can form on or near the ends of tightly-packed capillaries of the capillary array. FIG. 12A shows a portion of a capillary array (10) of the invention, to depict a situation in which a condensation bead (80) forms on the outer edge surface of several capillary walls (30), creating a potential conduit or bridge for “crosstalk” of matter between adjacent capillary tubes (20). The outer edge surface of the capillary walls (30) is preferably a planar surface. In an embodiment in which the wall (30) of the capillary (20) is glass, the outer edge surface of the capillary wall (30) can be polished glass.

In order to minimize the effects of such condensation, a hydrophobic coating (35) is provided over the outer edge surface of the capillary walls (30), as depicted in FIG. 12B. The coating (35) reduces the tendency for water or other liquid to accumulate near the outer edge surface of the capillary wall (30). Condensation will form either as smaller beads (82), be repelled from the surface of the capillary array, or form entirely over an opening to the lumen (40). In the latter case, the condensation bead (80) can form a cap to the capillary (20). In one embodiment, the hydrophobic coating (35) is TEFLON. In one configuration, the coating (35) covers only the outer edge surfaces of the capillary walls (30). In another configuration, the coating (35) can be formed over both the interstitial material (60) and the outer edge surfaces of the capillary walls (30). Another advantage of a hydrophobic coating (35) over the outer edge surface of the capillary tubes is during the initial wicking process, some fluidic material in the form of droplets will tend to stick to the surface in which the fluid is introduced. Therefore, the coating (35) minimizes extraneous fluid from forming on the surface of a capillary array (10), dispensing with a need to shake or knock the extraneous fluid from the surface.

In some instances, it is necessary to have more than one component in a capillary that are not premixed, and which can by later combined by dilution or mixing. FIGS. 13A-C show a dilution process that may be used to achieve a particular concentration of particles. In one embodiment employing dilution, a bolus of a first component (82) is wicked into a capillary (20) by capillary action until only a portion of the capillary (20) is filled. In one particular embodiment, pressure is applied at one end of the capillary (20) to prevent the first component from wicking into the entire capillary (20). The end (21) of the capillary may be completely or partially capped to provide the pressure.

An amount of air (84) is then introduced into the capillary adjacent the first component. The air (84) can be introduced by any number of processes. One such process includes moving the first component (82) in one direction within the capillary until a suitable amount of the air (84) is introduced behind the first component (82). Further movement of the first component (82) by a pulling and/or pushing pressure causes a piston-like action by the first component (82) on the air.

The capillary (20) or capillary array is then contacted to a second component (86). The second component (86) is preferably pulled into the capillary (20) by the piston-like action created by movement of the first component (82), until a suitable amount of the second component (86) is provided in the capillary, separated from the first component by the air (84). One of the first or second components may contain one or more particles of interest, and the other of the components may be a developer of the particles for causing an activity of interest. The capillary or capillary array can then be incubated for a period of time to allow the first and second components to reach an optimal temperature, or for a sufficient time to allow cell growth for example. The air-bubble separating the two components can be disrupted in order to allow mix the two components together and initialize the desired activity. Pressure can be applied to collapse the bubble. In one example, the mixture of the first and second components starts an enzymatic activity to achieve a multi-component assay.

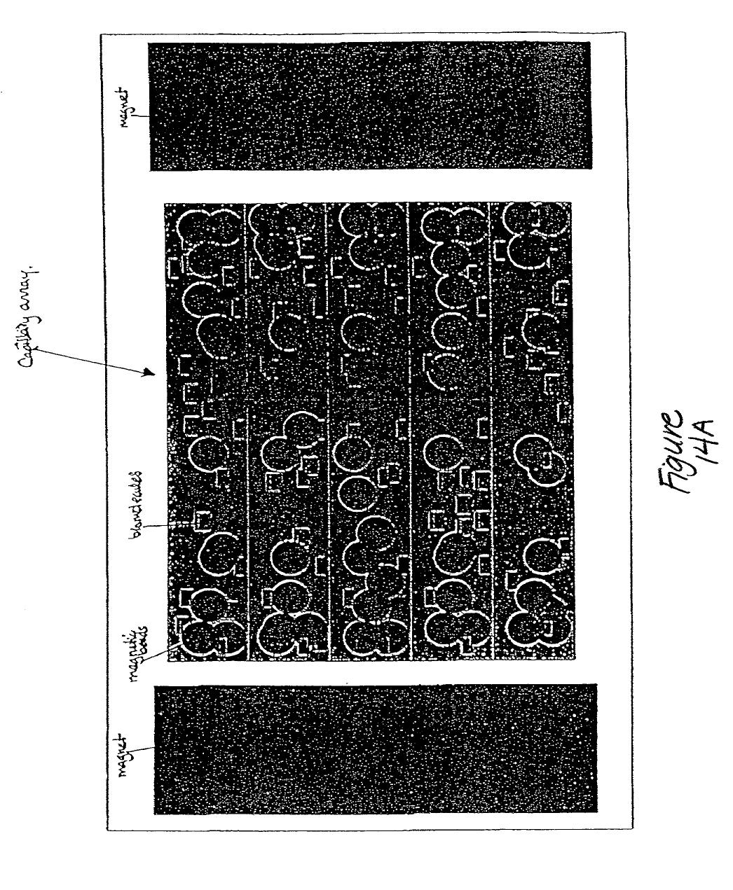

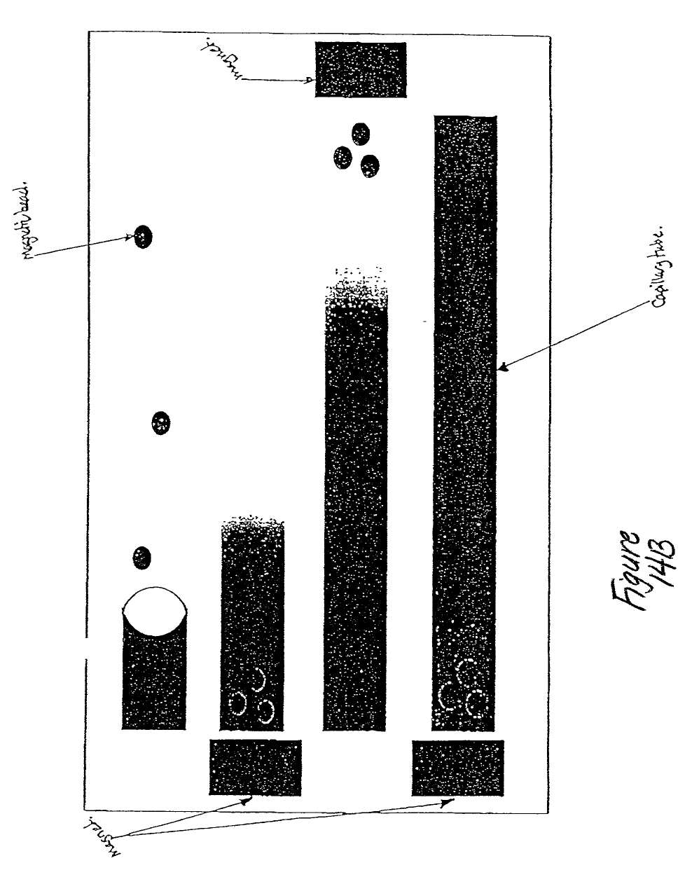

Paramagnetic beads contained within a capillary (20) can be used to disrupt the air bubble and/or mix the contents of the capillary (20) or capillary array (10). For example, FIG. 14A and 9B depict an embodiment of the invention in which paramagnetic beads are magnetically moved from one location to another location. The paramagnetic beads are attracted by magnetic fields applied in proximity to the capillary or capillary array. By alternating or adjusting the location of the magnetic field with respect to each capillary, the paramagnetic beads will move within each capillary to mix the liquid therein. Mixing the liquid can improve cell growth by increasing aeration of the cells. The method also improves consistency and detectability of the liquid sample among the capillaries.

In another embodiment, a method of forming a multi-component assay includes providing one or more capsules of a second component within a first component. The second component capsules can have an outer layer of a substance that melts or dissolves at a predetermined temperature, thereby releasing the second component into the first component and combining particles among the components. A thermally activated enzyme may be used to dissolve the outer layer substance. Alternatively, a “release on command” mechanism that is configured to release the second component upon a predetermined event or condition may also be used.

In another embodiment, recombinant clones containing a reporter construct or a substrate are wicked into the capillary tubes of the capillary array. In this embodiment, it is not necessary to add a substrate as the reporter construct or substrate contained in the clone can be readily detected using techniques known in the art. For example, a clone containing a reporter construct such as green fluorescent protein can be detected by exposing the clone or substrate within the clone to a wavelength of light that induces fluorescence. Such reporter constructs can be implemented to respond to various culture conditions or upon exposure to various physical stimuli (including light and heat). In addition, various compounds can be screened in a sample using similar techniques. For example, a compound detectably labeled with a florescent molecule can be readily detected within a capillary tube of a capillary array.

In yet another embodiment, instead of dilution, a fluorescence-activated cell sorter (FACS) is used to separate and isolate clones for delivery into the capillary array. In accordance with this embodiment, one or more clones per capillary tube can be precisely achieved. In yet another embodiment, cells within a capillary are subjected to a lysis process. A chemical is introduced within one of the components to cause a lysis process where the cells burst.

Some assays may require an exchange of media within the capillary. In a media exchange process, a first liquid containing the particles is wicked into a capillary. The first liquid is removed, and replaced with a second liquid while the particles remain suspended within the capillary. Addition of the second liquid to the capillary and contact with the particles can initialize an activity, such as an assay, for example. The media exchange process may include a mechanism by which the particles in the capillary are physically maintained in the capillary while the first liquid is removed. In one embodiment, the inner walls of the capillary array are coated with antibodies to which cells bind. Then, the first liquid is removed, while the cells remain bound to the antibodies, and the second liquid is wicked into the capillary. The second liquid could be adapted to cause the cells to unbind if desirable. In an alternative embodiment, one or more walls of the capillary can be magnetized. The particles are also magnetized and attracted to the walls. In still another embodiment, magnetized particles are attracted and held against one side of the capillary upon application of a magnetic field near that side.

The capillary array is analyzed for identification of capillaries having a detectable signal, such as an optical signal (e.g., fluorescence), by a detector capable of detecting a change in light production or light transmission, for example. Detection may be performed using an illumination source that provides fluorescence excitation to each of the capillaries in the array, and a photodetector that detects resulting emission from the fluorescence excitation. Suitable illumination sources include, without limitation, a laser, incandescent bulb, light emitting diode (LED), arc discharge, or photomultiplier tube. Suitable photodetectors include, without limitation, a photodiode array, a charge-coupled device (CCD), or charge injection device (CID).

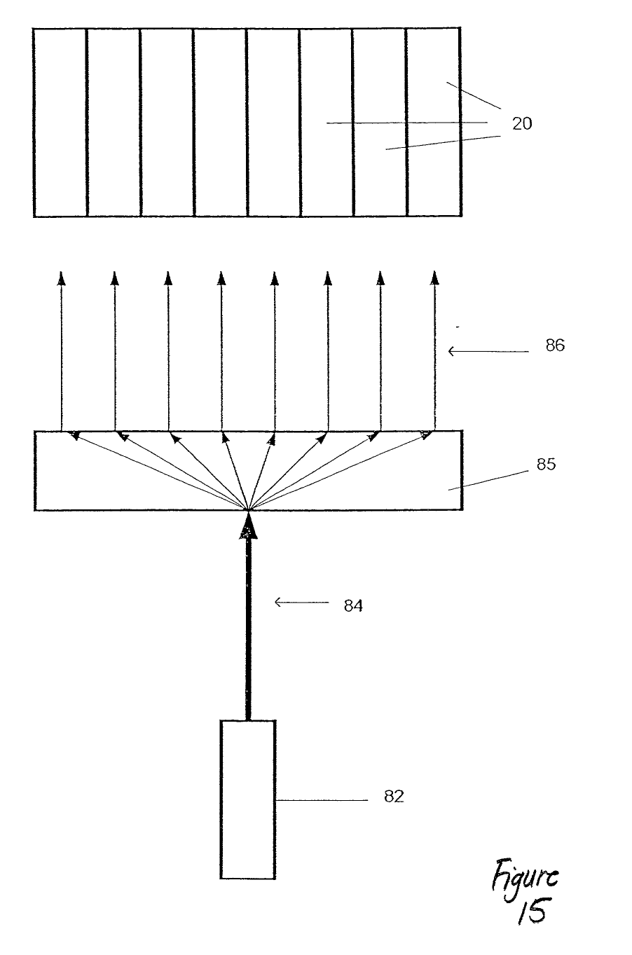

In one embodiment, shown with reference to FIG. 15, a detection system includes a laser source (82) that produces a laser beam (84). The laser beam (84) is directed into a beam expander (85) configured to produce a wider or less divergent beam (86) for exciting the array of capillaries (20). Suitable laser sources include argon or ion lasers. For this embodiment, a cooled CCD can be used.

The light generated by, for example, enzymatic activation of a fluorescent substrate is detected by an appropriate light detector or detectors positioned adjacent to the apparatus of the invention. The light detector may be, for example, film, a photomultiplier tube, photodiode, avalanche photo diode, CCD or other light detector or camera. The light detector may be a single detector to detect sequential emissions, such as a scanning laser. Or, the light detector may include a plurality of separate detectors to detect and spatially resolve simultaneous emissions at single or multiple wavelengths of emitted light. The light emitted and detected may be visible light or may be emitted as non-visible radiation such as infrared or ultraviolet radiation. A thermal detector may be used to detect an infrared emission. The detector or detectors may be stationary or movable.

Illumination can be channeled to particles of interest within the array by means of lenses, mirrors and fiber optic light guides or light conduits (single, multiple, fixed, or moveable) positioned on or adjacent to at least one surface of the capillary array. A detectable signal, such as emitted light or other radiation, may also be channeled to the detector or detectors by the use of such mechanisms.

The photodetector preferably comprises a CCD, CID or an array of photodiode elements. Detection of a position of one or more capillaries having an optical signal can then be determined from the optical input from each element. Alternatively, the array may be scanned by a scanning confocal or phase-contrast fluorescence microscope or the like, where the array is, for example, carried on a movable stage for movement in a X-Y plane as the capillaries in the array are successively aligned with the beam to determine the capillary array positions at which an optical signal is detected. A CCD camera or the like can be used in conjunction with the microscope. The detection system is preferably computer-automated for rapid screening and recovery. In a preferred embodiment, the system uses a telecentric lens for detection. The magnification of the lens can be adjusted to focus on a subset of capillaries in the capillary array. At one extreme, for instance, the detection system can have a 1:1 correlation of pixels to capillaries. Upon detecting a signal, the focus can be adjusted to determine other properties of the signal. Having more pixels per capillary allows for subsequent image processing of the signal.

Where a chromogenic substrate is used, the change in the absorbance spectrum can be measured, such as by using a spectrophotometer or the like. Such measurements are usually difficult when dealing with a low-volume liquid because the optical path length is short. However, the capillary approach of the present invention permits small volumes of liquid to have long optical path lengths (e.g., longitudinally along the capillary tube), thereby providing the ability to measure absorbance changes using conventional techniques.

A fluid within a capillary will usually form a meniscus at each end. Any light entering the capillary will be deflected toward the wall, except for paraxial rays, which enter the meniscus curvature at its center. The paraxial rays create a small bright spot in middle of capillary, representing the small amount of light that makes it through. Measurement of the bright spot provides an opportunity to measure how much light is being absorbed on its way through. In a preferred embodiment, a detection system includes the use of two different wavelengths. A ratio between a first and a second wavelength indicates how much light is absorbed in the capillary. Alternatively, two images of the capillary can be taken, and a difference between them can be used to ascertain a differential absorbance of a chemical within the capillary.

In absorbance detection, only light in the center of the lumen can travel through the capillary. However, if at least one meniscus is flattened, the optical efficiency is improved. The meniscus can be kept flat under a number of circumstances, such as during a continuous cycle of evaporation, discussed above with reference to FIG. 11. In that embodiment, the fluid bath can be contained in a clear, light-passing container, and the light source can be directed through the fluid bath into the capillary.

In another embodiment, bioactivity or a biomolecule or compound is detected by using various electromagnetic detection devices, including, for example, optical, magnetic and thermal detection. In yet another embodiment, radioactivity can be detected within a capillary tube using detection methods known in the art. The radiation can be detected at either end of the capillary tube.

Other detection modes include, without limitation, luminescence, fluorescence polarization, time-resolved fluorescence. Luminescence detection includes detecting emitted light that is produced by a chemical or physiological process associated with a sample molecule or cell. Fluorescence polarization detection includes excitation of the contents of the lumen with polarized light. Under such environment, a fluorophore emits polarized light for a particular molecule. However, the emitting molecule can be moving and changing its angle of orientation, and the polarized light emission could become random.

Time-resolved fluorescence includes reading the fluorescence at a predetermined time after excitation. For a relatively long-life fluorophore, the molecule is flashed with excitation energy, which produces emissions from the fluorophore as well as from other particles within the substrate. Emissions from the other particles causes background fluorescence. The background fluorescence normally has a short lifetime relative to the long-life emission from the fluorophore. The emission is read after excitation is complete, at a time when all background fluorescence usually has short lifetime, and during a time in which the long-life fluorophores continues to fluoresce. Time-resolved fluorescence are therefore a technique for suppressing background fluorescent activity.

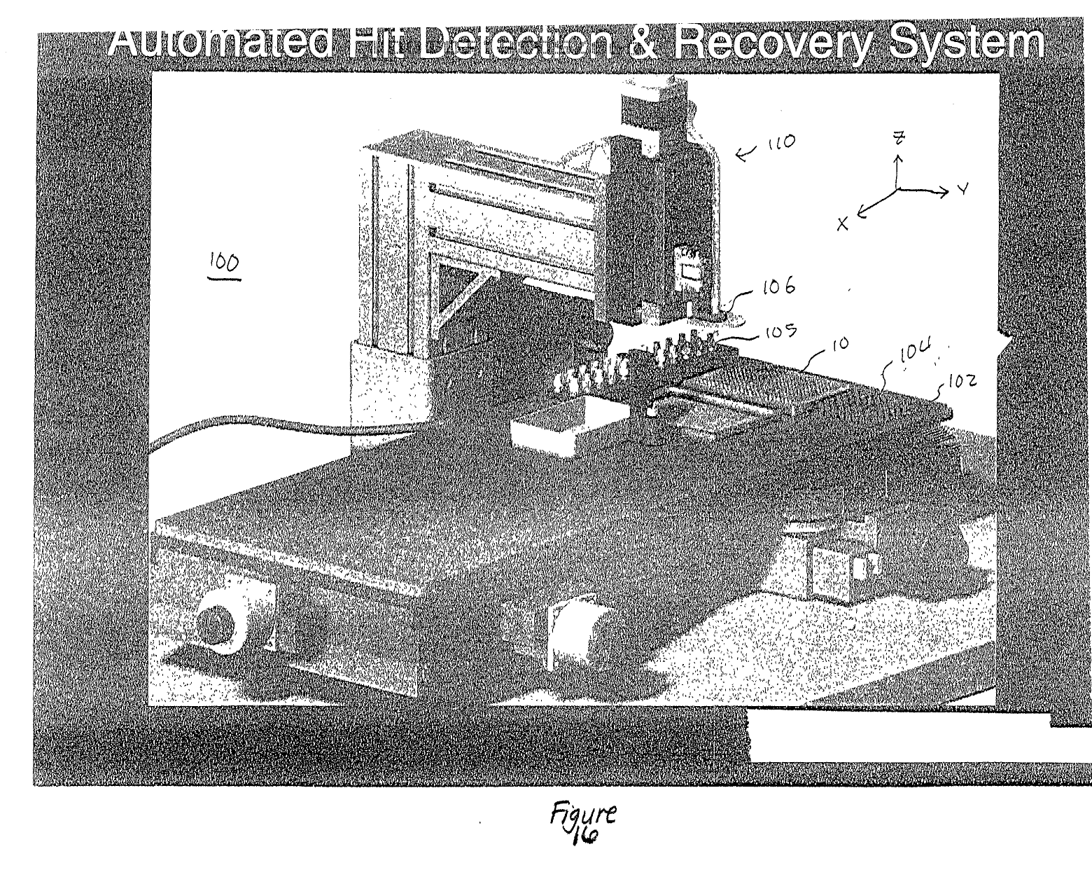

Recovery of putative hits (cells or clones producing a detectable or optical signal) can be facilitated by using position feedback from the detection system to automate positioning of a recovery device (e.g., a needle pipette tip or capillary tube). FIG. 16 shows an example of a recovery system (100) of the invention. In this example, a needle 105 is selected and connected to recovery mechanism (106). A support table (102) supports a capillary array (10) and a light source (104). The light source is used with a camera assembly (110) to find an X, Y and Z coordinate location of a needle (105) connected to the recovery mechanism (106). The support table is moved relative to the capillary array in the X and Y axes, in order to place the capillary array (10) underneath the needle (105), where the capillary array (10) contains a “hit.” According to various embodiments, each section of a recovery system can be moved or kept stationary.

The recovery mechanism (106) then provides a needle (105) to a capillary containing a “hit” by overlapping the tip of the needle (105) with the capillary containing the “hit,” in the Z direction, until the tip of the needle engages the capillary opening. In order to avoid damage to the capillary itself the needle may be attached to a spring or be of a material that flexes. Once in contact with the opening of the capillary the sample can be aspirated or expelled from the capillary. Alternatively, the capillary array may be moved relative to a stationary needle (105), or both moved.

In a specific exemplary embodiment of a recovery technique, a single camera is used for determining a location of a recovery tool, such as the tip of a needle, in the Z-plane. The Z-plane determination can be accomplished using an auto-focus algorithm, or proximity sensor used in conjunction with the camera. Once the proximity of the recovery tool in Z is known, an image processing function can be executed to determine a precise location of the recovery tool in X and Y. In one embodiment, the recovery tool is back-lit to aid the image processing. Once the X and Y coordinate locations are known, the capillary array can be moved in X and Y relative to the precise location of the recovery tool, which can be moved along the Z axis for coupling with a target capillary.

In an alternative specific embodiment of a recovery technique, two or more cameras are used for determining a location of the recovery tool. For instance, a first camera can determine X and Z coordinate locations of the recovery tool, such as the X, Z location of a needle tip. A second camera can determine Y and Z coordinate locations of the recovery tool. The two sets of coordinates can then be multiplexed for a complete X,Y,Z coordinate location. Next, the movement of the capillary array relative to the recovery tool can be executed substantially as above.

The sample can be expelled by, for example, injecting a blast of inert gas or fluid into the capillary and collecting the ejected sample in a collection device at the opposite end of the capillary. The diameter of the collection device can be larger than or equal to the diameter of the capillary. The collected sample can then be further processed by, for example, extracting polynucleotides, proteins or by growing the clone in culture.

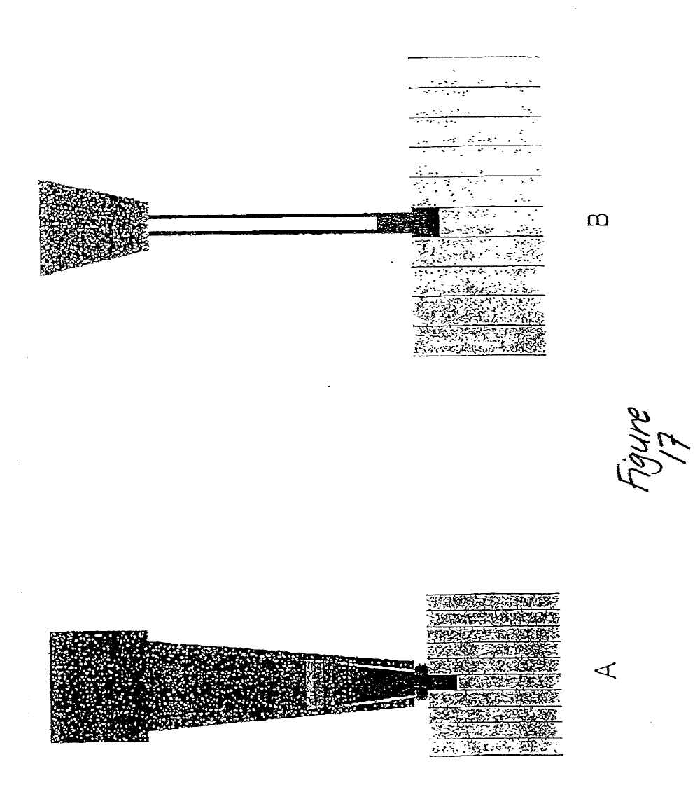

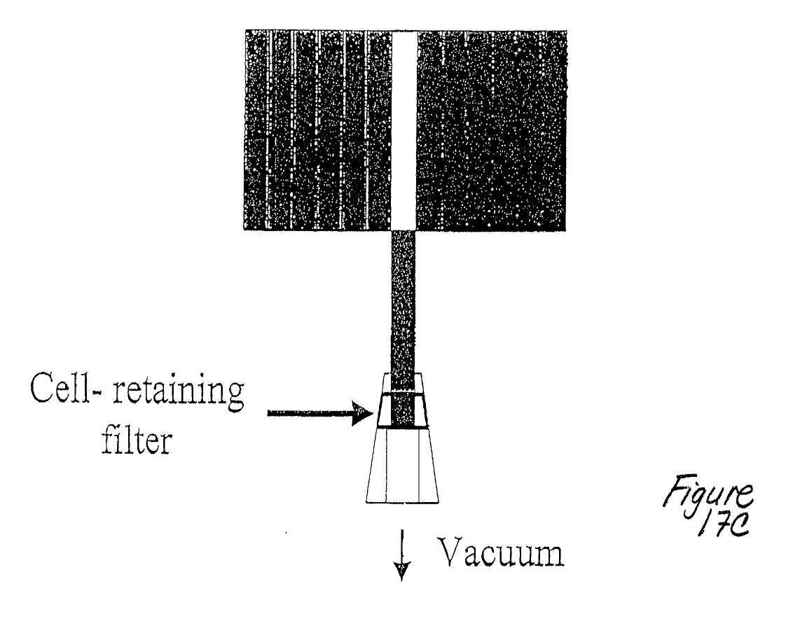

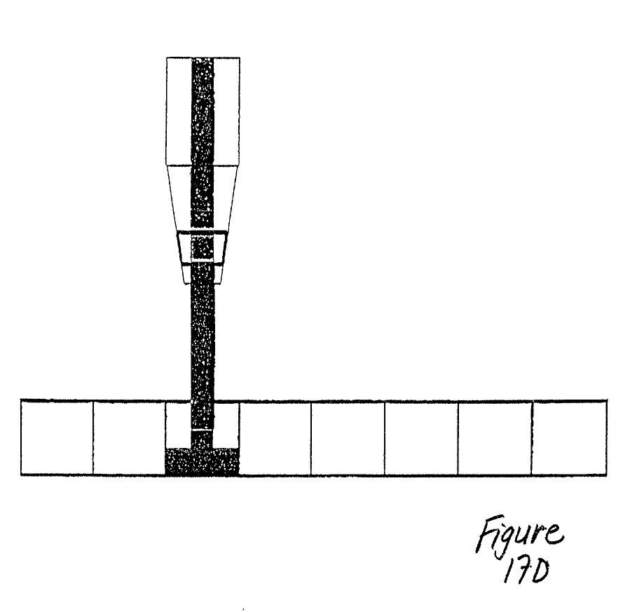

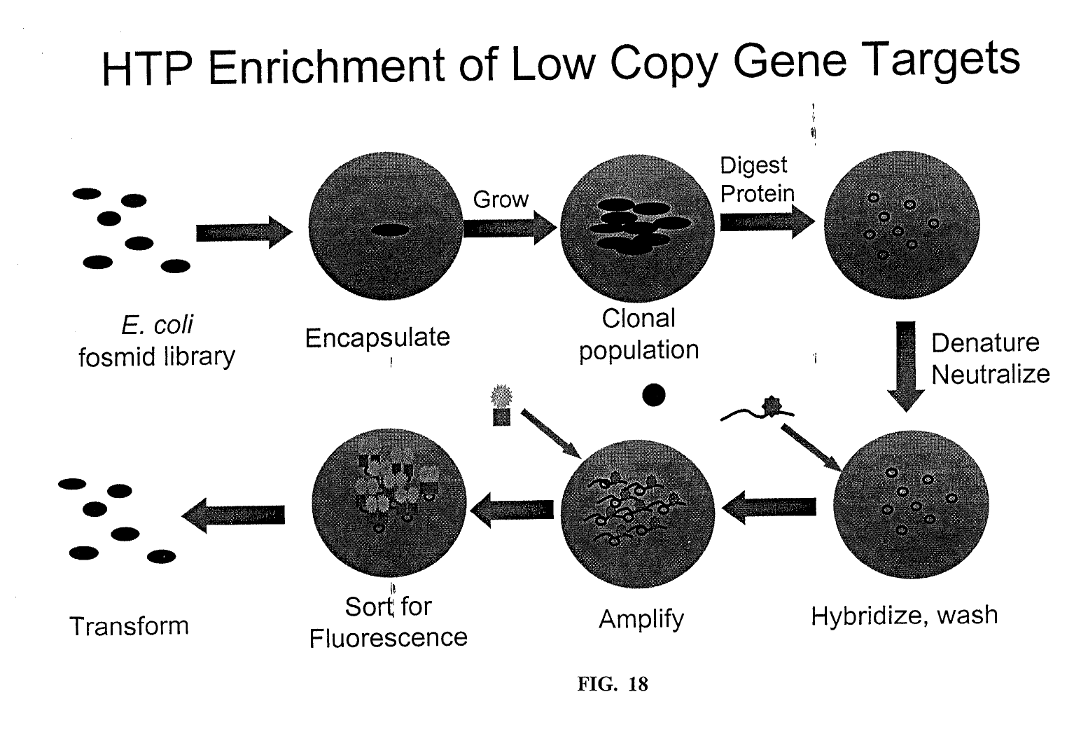

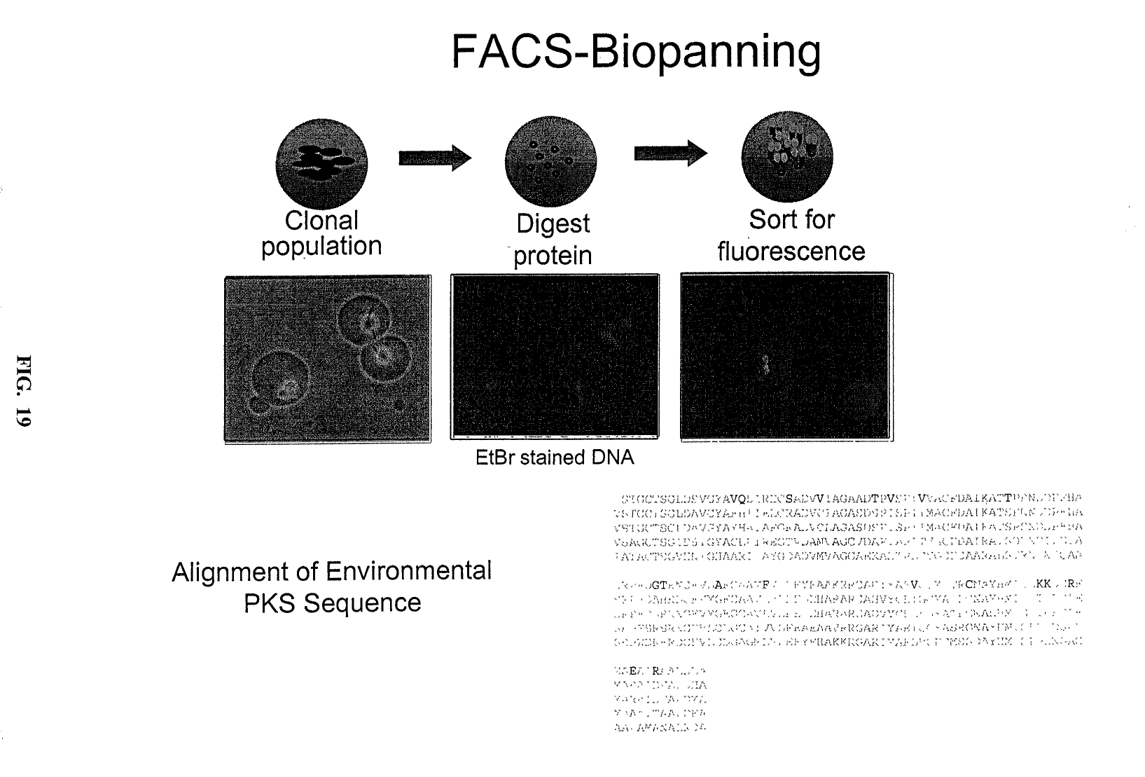

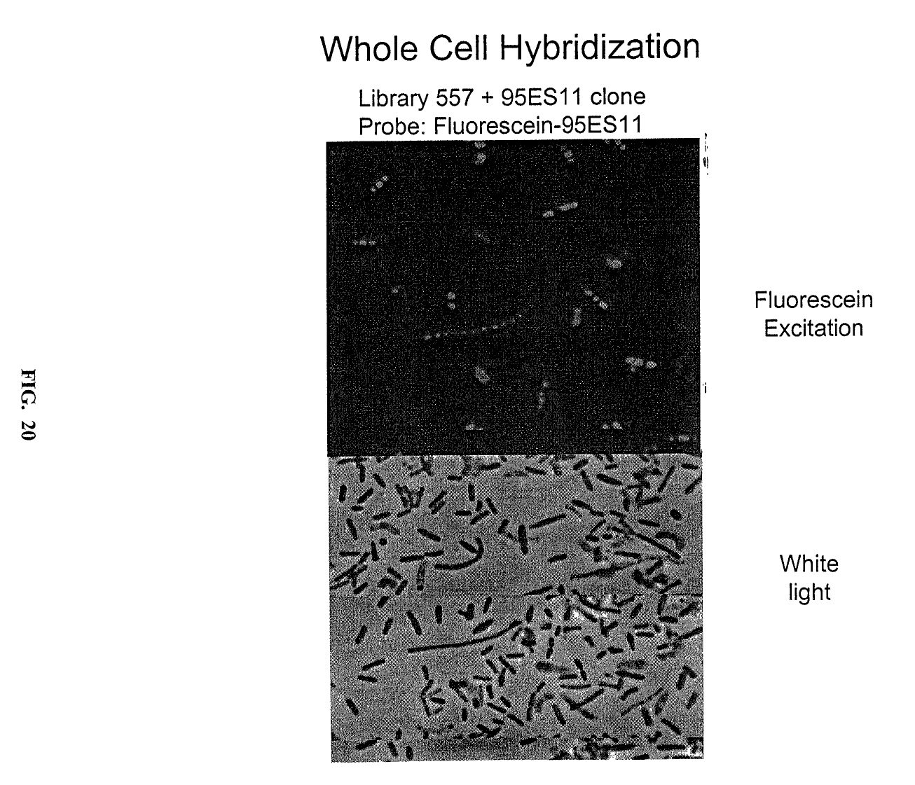

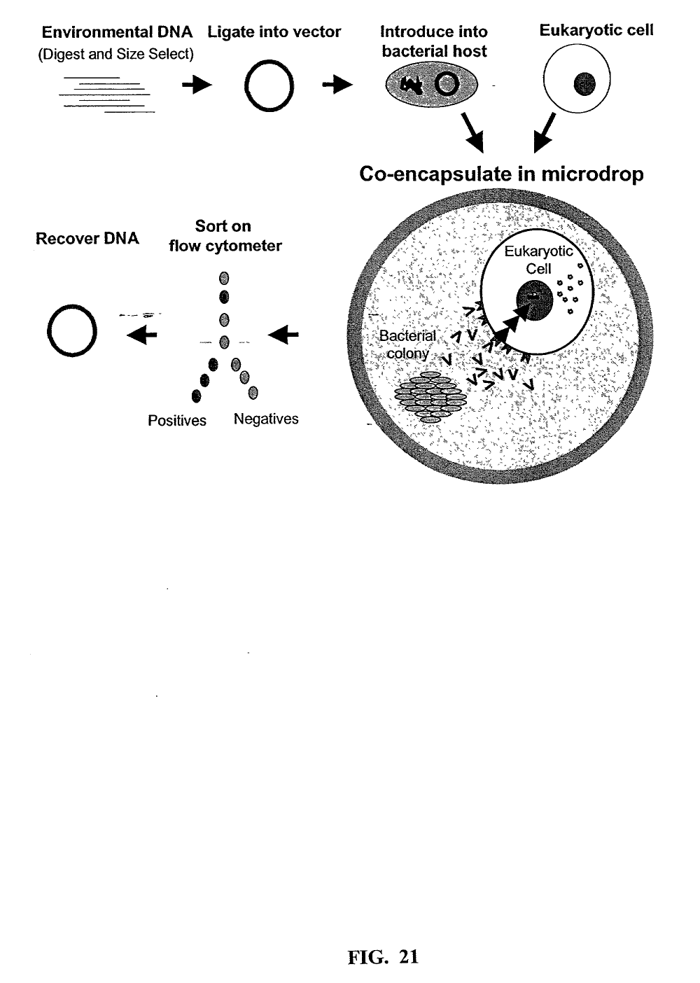

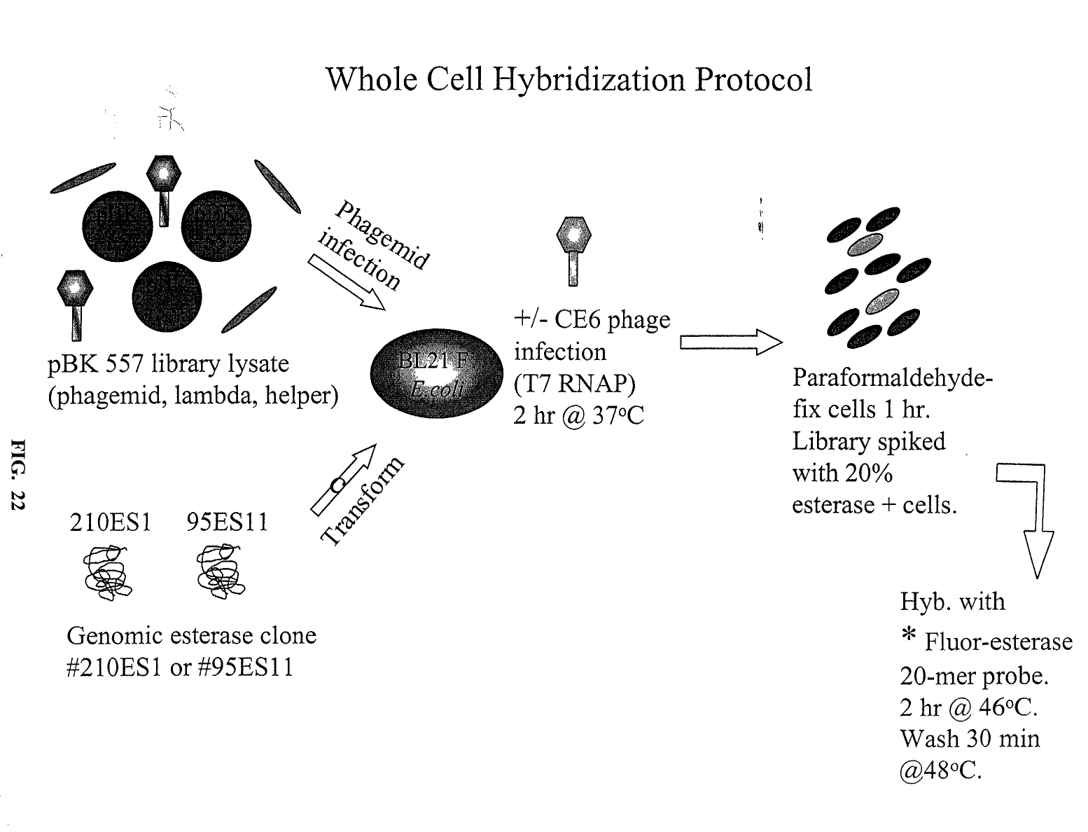

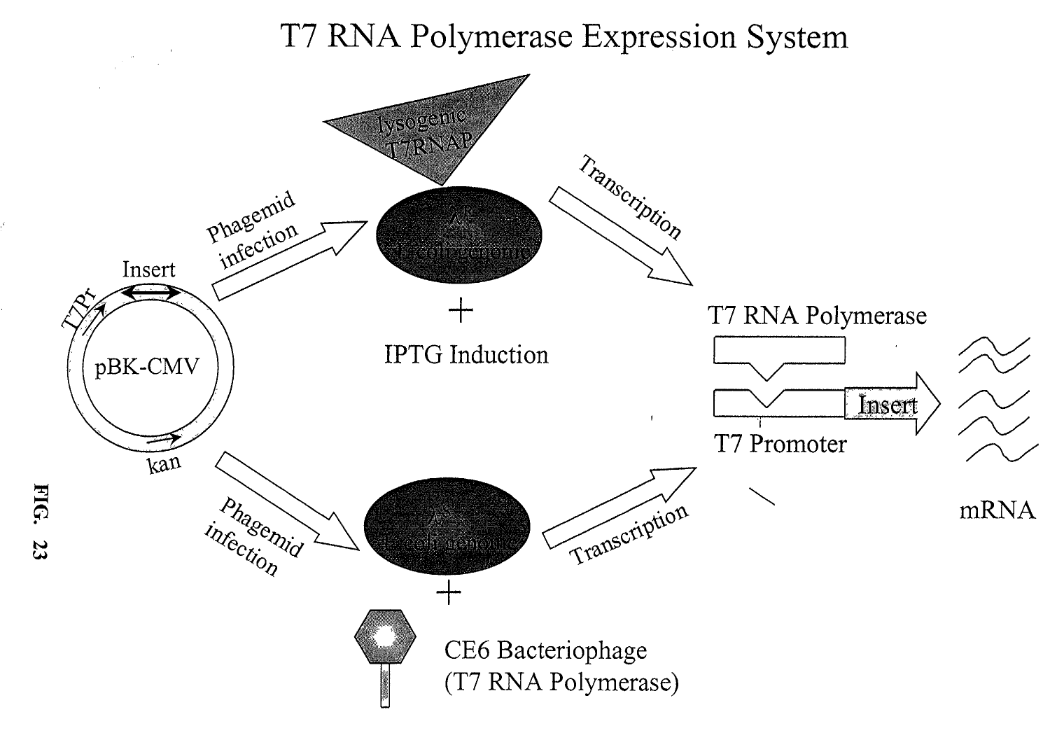

In another embodiment, the sample is aspirated by use of a vacuum. In this embodiment, the needle contacts, or nearly contacts, the capillary opening and the sample is “vacuumed” or aspirated from the capillary tube onto or into a collection device. The collection device may be a microfuge tube or a filter located proximal to the opening of the needle, as depicted in FIGS. 17A-D. FIG. 17D shows further processing of a sample collected onto a filter following aspiration of the sample from the capillary. The sample includes particles, such as cells, proteins, or nucleic acids, which when present on the filter, can be delivered into a collection device. Suitable collection devices include a microfuge tube, a capillary tube, microtiter plate, cell culture plate, and the like. The delivery of the sample can be accomplished by forcing another media, air or other fluid through the filter in the reverse direction.

The sample can also be expelled from a capillary by a sample ejector. In one embodiment, the ejector is a jet system where sample fluid at one end of the capillary tube is subjected to a high temperature, causing fluid at the other end of the capillary tube to eject out. The heating of fluid can be accomplished mechanically, by applying a heated probe directly into one end of a capillary tube. The heated probe preferably seals the one end, heats fluid in contact with the probe, and expels fluid out the other end of the capillary tube. The heating and expulsion may also be accomplished electronically. For instance, in an embodiment of the jet system, at least one wall of a capillary tube is metalized. A heating element is placed in direct contact with one end of the wall. The heating element may completely close off the one end, or partially close the one end. The heating element charges up the metalized wall, which generates heat within the fluid. The heating element can be an electricity source, such as a voltage source, or a current source. In still yet another embodiment of a jet system, a laser applies heat pulses to the fluid at one end of the capillary tube.

Other systems for expelling fluid from a capillary tube of the invention are possible. An electric field may be created in or near the fluid to create an electrophoretic reaction, which causes the fluid to move according to electromotive force created by the electric field. A electromagnetic field may also be used. In one embodiment, one or more capillaries contain, in addition to the fluid, magnetically charged particles to help move the fluid or magnetized partcles out of the capillary array.

Each capillary of an array of capillaries is individually addressable, i.e. the contents of each well can be ascertained during screening. In one embodiment, a quantum-dot-tagged microbead method and arrangement is used. In such a method and arrangement, tens of thousands of unique fluorescent codes can be generated. The assay of interest is attached to a coded bead, and multi-spectral imaging is used to measure both the assay and the beads/codes simultaneously. There will always be some capillaries that get multiple beads and some that get none.

For an array which contains approximately 100,000 capillaries, one approach is to fill the 100,000 capillaries of the array with a solution that contains 10 copies of 10,000 different coded beads (or 5 copies of 20,000 codes). Under normal conditions, simple statistical analysis can be used to determine which of the wells have single beads and maybe even the contents of every well. The chance of having any two beads together in a well more than 5 times on any one capillary array platform is negligibly small.

An advantage of the quantum-dots method is that only a single excitation band is needed. This allows a lot of flexibility for the assay (i.e. it can use a different excitation band). Magnetic-coded beads may also be used to add another dimension to the assay detection. A multi-spectral imaging system can then be used. Alternatively, a neural network application can be utilized for spectral decomposition.

The myriad of microbes inhabiting this planet represent a tremendous repository of biomolecules for pharmaceutical, agricultural, industrial and chemical applications. The great majority of these microbes, estimated at near 99.5%, have remained uncultured by modem microbiological methods due in large part to the complex chemistries and environmental variables encountered in extreme or unusual biotopes. Taking advantage of enzymes catalyzing chemical reactions in novel pathways and evolved to function under environmental extremes is of great industrial significance. This invention provides technologies to extract, optimize and commercialize this robust catalytic diversity, within culture-independent, recombinant approaches for the discovery of novel enzymes and biosynthetic pathways by tapping into the biodiversity present in nature. Large, complex (>109 member) gene libraries are constructed by direct isolation of DNA from selected microenvironments around the world. These libraries are then expressed in various host systems and subjected to high throughput screens specific for an activity of interest. Because in excess of 5000 different microbial genomes may be present in a single DNA library, ultra high throughput methods are required to effectively screen this diversity and are crucial to the success of this culture-independent, recombinant strategy.

Conventional screening methods include liquid phase, microtiter plate based assays. The format for liquid phase assays is often robotically manipulated 96, 384, or 1536-well microtiter plates. Although these microtiter plate based screening technologies are being used successfully, limitations do exist. The primary limitation is throughput as these techniques generally allow the screening of only about 105 to 106 clones/day/instrument. For example, a typical screen of 100,000 wells on a microtiter based HTS systems requires 261,384-well microtiter plates and over 24 hours of equipment time. However, while 1536-well or greater plate formats are growing in popularity, the majority of companies involved in HTS continue to use 384-well plates, as this technology is reliable and standardized. While these throughputs may be more than sufficient for screening isolate and low-complexity libraries, it could take more than a year to thoroughly screen one complex gene library. Clearly, higher throughput screening technology is necessary.

Other screening methods include growth selection (Snustad et al., 1988; Lundberg et al., 1993; Yano et al., 1998), calorimetric screening of bacterial colonies or phage plaques (Kuritz, 1999), in vitro expression cloning (King et al., 1997) and cell surface or phage display (Benhar, 2001). Each of these systems has limitations. Solid phase calorimetric plate screening of colonies or plaques is limited by relatively low throughput. Even with the use of microcolonies/plaques and automated imaging and clone recovery, thorough screening of complex libraries is impractical. Cell surface and/or phage display technologies suffer from structural limitations of the displayed molecule. Often the size and/or shape of the displayed molecule is restricted by the display technology. One of the highest throughput screening methods, growth selection, is also limited in its scope of usefulness. Assay conditions, temperature and pH, are limited by the growth parameters of the host strain. Molecular interactions are often constrained by the host cell membranes and/or cell wall, as substrate must be presented to intracellular enzymes. In addition, “false positives” or a high level of “background” are a common occurrence in many selection assays. With respect to screening for improved variants in GSSM or GeneReassembly libraries, growth selection is seldom quantitative.

The invention provides screening platforms and methods for use with a Fluorescence Activated Cell Sorter (FACS). In FACS methodologies, cells are mixed with substrates and then streamed past a detector to screen for a positive molecular event. This signal could be a fluorescent signal resulting from the cleavage of an enzyme substrate or a specific binding event. The greatest advantage of the use of a FACS machine is throughput; up to 109 clones can be screened/day. Unfortunately, FACS based screening also has limitations including cell wall permeability of enzymes and substrates/products and incubation times and temperatures. In addition, viability of host cells post-sort and dependence on a single data point for each individual cell further limit such technologies.

The development of the capillary array overcomes many of these shortcomings. Like microtiter and solid phase screens, it combines the preservation of native protein conformation with increased signal strength of clonal amplification. The throughput, however, approaches that of selective assays and FACS-based assays. Moreover, as array plates are reusable, the amount of plastic waste generated is greatly reduced. Approximately 24 tons of plastic waste* is generated annually in screening 100,000 wells per day in a 96 well format (* Assuming 84 g/plate×1000 plates/day×260 days/year). Further, a typical screen of 100,000 wells on a robotic high throughput screening system requires 261 384-well microtiter plates and over 24 hours of equipment time versus less than 10 minutes to process a single plate. The enhancement of this technology to densities of one million wells per plate is aimed at approaching the throughput of selective assays and FACS-based assays while retaining the advantages of a microtiter-based screen.

The first generation capillary array plates can be fabricated using manufacturing techniques originally developed for the fiber optics industry, currently consist of 100,000 cylindrical compartments or wells contained within a 3.3″×5″ reusable plate, the size of a SBS (Society for Biomolecular Screening) standard 96 well microtiter plate. These wells are 200 μm in diameter (about the diameter of a human hair) and act as discrete 250 nanoliter volume microenvironments in which isolated clones can be grown and screened.

The processes involved in array screening closely parallel those in microtiter plate screening, but with significant simplification in required instrumentation and decrease in plate storage capacity requirements and reagent costs. Briefly, the plates are filled with clones and reagents (e.g. fluorescent substrate, growth media, etc.) by surface tension, filling all 100,000 wells simultaneously within a few seconds without the need for complicated dispensing equipment. The number of clones per well, typically 1 to 10, is adjusted by dilution of the cell culture. Once filled, the plates are then incubated in a humidity-controlled environment for 24 to 48 hours to allow for both clonal amplification and enzymatic turnover.

After incubation in a humidified chamber, the plates are transferred to the detection and recovery station where fluorescence imaging is used to detect the expression of bioactive molecules. The automated detection and recovery system combines fluorescence imaging and precision motion control technologies through the use of machine vision and image processing techniques. Images are generated by focusing light from a broadband light source (e.g. metal halide arc lamp) onto the plate through a set of fluorescence excitation filters. The resulting fluorescence emission is filtered then imaged by a telecentric lens onto a high-resolution cooled CCD camera in an epi-fluorescent configuration. The plates are scanned to generate a total of 56 slightly overlapping images in approximately one minute. The images are digitized and processed on-the-fly to detect and locate positive wells or putative hits. Putative hits (clones that have converted the substrate to a fluorescent product) appear as bright spots on a dark background. They are distinguished from background fluorescence and extraneous signals (typically due to dirt and dust) based on a variety of feature measurements such as their shape, size, and intensity profile.

Once detected and located, putative hits are recovered from the array plate and transferred to a standard microtiter plate for confirmation and secondary screening. The process of recovery consists of: 1) mounting and locating a sterile recovery needle (typically a standard blunt end stainless steel needle commonly used for dispensing adhesives for mounting miniature surface mount electronic components), 2) aligning the recovery needle to the well containing the putative hit, 3) aspirating the contents of the well into the needle (which has attached 0.22 micron filter to avoid upstream contamination and loosing the sample), 4) flushing the well contents into a standard microtiter plate with an appropriate media, and finally 5) stripping off the recovery needle in preparation for the next recovery. Closed loop positioning with image-based feedback provides the positional accuracy required to allow aspiration of individual wells without contamination from neighboring wells. Finally, after the clones of interest have been recovered, the used plates are cleaned, sterilized, and prepared for re-use. The array platform according to the invention will accelerate the discovery and development of commercial products as well as enable the development of products that would otherwise be unobtainable.

This invention is configured for use with a Fluorescence Activated Cell Sorter (FACS). In FACS methodologies, cells are mixed with substrates and then streamed past a detector to screen for a positive molecular event. This signal could be a fluorescent signal resulting from the cleavage of an enzyme substrate or a specific binding event. The greatest advantage of the use of a FACS machine is throughput; up to 109 clones can be screened/day. Unfortunately, FACS based screening also has limitations including cell wall permeability of enzymes and substrates/products and incubation times and temperatures. In addition, viability of host cells post-sort and dependence on a single data point for each individual cell further limit such technologies.

The well diameter, plate thickness (well depth), and material optical properties will be specified prior to fabricating the new 1,000,000-well density matrices. Once these parameters are specified, high density matrices will be fabricated in rectangular pieces approximately 1 cm square. The process entails a low-risk modification to the same basic fabrication technique that is used to make the 100,000 well plates. The array density can be calculated by using the following formula:

$$\# WellsPerPlate = \frac{2}{\sqrt{3}} \frac{( PlateLength \times PlateWidth )}{{( WellDiameter + WellSeparationWall )}^{2}}$$

This calculation reveals that in order to achieve 1,000,000 wells in the standard 3.3″×5″ microtiter plate format, the new wells will need to have a diameter of approximately 70 μm with 25 μm separating walls. Structures of this size/density and smaller (down to 6 μm) are commonly manufactured for non-biological uses including micro-channel faceplates for intensified CCD cameras, X-ray scintillation plates, optical collimators, as well as simple fluid filters.

There are some limitations to the depth of the wells due to the nature of the fabrication process. The current 100,000-well plates have 8 mm deep wells. Based on our experience with structures of similar size, it is estimated that the depth of the 70 μm wells will be between 5 mm and 8 mm. This yields a well volume of approximately 25 nl to 30 nl or approximately 1/10th of that of the 200 μm diameter wells. Evaporation rate is a function of the surface area to volume ratio rather than the total volume. For this reason it is anticipated that the 70 μm wells will experience comparable (if not less) evaporation than the 200 μm well due to a more favorable length to diameter (volume to surface area) ratio. Evaporation is currently not a problem with the 200 μm diameter wells.

Samples will be constructed from both transparent and opaque materials to evaluate illumination efficiencies, well-to-well optical crosstalk, surface-finish effects, and background fluorescence. The current 100,000-well plates use an opaque material. The use of transparent materials improves the efficiency of fluorescence excitation at the expense of increased well-to-well optical crosstalk. For assays with low hit rates, the tradeoff may favor the use of transparent materials to improve detection sensitivity. We estimate that the specification and manufacturing process will take two months. A special holder will also be fabricated to adapt the matrices to the capillary array hardware. Once the specified matrices are manufactured, they will be tested for each of the optical and mechanical properties detailed below:

Background Fluorescence

It is helpful from an imaging and processing perspective, but not critical, that the matrix have low background fluorescence for a broad range of excitation wavelengths to allow use with a variety of substrates. The materials used in the 200 μm plates were tested and selected to satisfy this requirement. In the unlikely event that different materials must be used to fabricate both transparent and opaque 70 μm matrices, they will be tested for their fluorescent properties prior to fabrication. These tests are performed by measuring and comparing the fluorescence of the material to a reference standard at a range of excitation wavelengths.

Optical Efficiency

The 100,000-well plates are currently illuminated by a roughly collimated beam directly on the face of the plate. Light enters each well through the aperture formed by the wall around the well. Transparent materials are expected offer illumination advantages over opaque materials with the current illumination system by transmitting additional excitation energy through the walls separating the wells. The optical efficiency of the 1,000,000-well density matrices will be evaluated by determining the detectable concentration of a fluorescein solution. Typically, liquid phase enzyme discovery assays use 10-100 μM concentrations of fluorescent substrate. The current detection system can detect approximately 10 nM of fluorescein in the 200 μm wells. The equivalent fluorescence of LB (our typical cell growth media) is approximately 25 nM. Hardware modifications described in Goal 3 may be required in the unlikely event that the detectable levels are less than 10 μM for the new matrices.

Optical Crosstalk

While the use of transparent materials may improve the efficiency of fluorescence excitation as described above, it does so at the expense of increased well-to-well optical crosstalk. This optical crosstalk is due to fluorescence emission that leaks from one well into its neighbors. This is easily quantified by, spotting a fluorophore onto the matrix, and then measuring the signal intensity vs. distance from a fluorophore filled well. The crosstalk could potentially mask the signal of a weak positive well resulting in a false negative or be detected as a false positive. In applications where the expected hit rate is low (which is commonly the case with enzyme discovery from environmental libraries) the probability of this occurring is generally insignificant. However, crosstalk can complicate the image processing required to automatically locate putative hits and therefore must be evaluated.

Surface Tension/Wicking Properties

The plates are filled by placing the surface of the plate in contact with the assay solution. Surface tension at the liquid/plate interface causes the assay components to be drawn or wick into all of the wells simultaneously. The surface preparation of the plate can have significant affects on the wicking properties of the matrix. Some surface polishing techniques have been found to make the glass face of the plate hydrophobic, thus preventing or significantly slowing the filling of the plate. Initially, the same surface finish currently used on the 100,000-well plate will be tested. If necessary, matrices with different surface preparations will be placed into contact with a cell/media mixture and their wicking properties quantified by timing the filling process and weighing the matrices before and after filling. In the event that plate filling remains inadequate after testing available surface preparations and treatments, surfactants can be added to improve filling.

Resistance to Cleaning and Sterilization

It is desirable for the 1,000,000-well plates to be reusable. To validate this requirement, the matrices will be processed through multiple, rigorous cleaning and sterilization protocols. Currently, there is a great deal of latitude in both the cleaning and sterilization protocols. Cleaning can consist of a combination of flushing, soaking, and/or sonication in water, solvents and/or soaps. Likewise, due to the inherent ruggedness of the materials used, sterilization can be accomplished by autoclaving, bleach, ethanol, and/or acid washing. Cleanliness is verified by fluorescence imaging of the material at multiple excitation wavelengths. Sterilization is verified by overnight incubation of matrices filled with sterile growth media, followed by plating the contents onto agar and looking for colony formation.

Only minimal modifications to the detection system hardware will be required for the 1,000,000-well density matrices. Due to reduced size of the wells, minor modifications to the optical system may need to be made to adjust the magnification to an appropriate level to determine screening feasibility. The optical system will likely need further modification as proposed in Phase II to enable automated hit recovery. A commercially available 2× extender can be added to the existing telecentric imaging lens used for the current 100,000-well plate. This modification will render the final image size of each well (relative to the camera) approximately 70% of the current size. Based on our experience, this should be more than adequate to visualize positive wells for determining feasibility.

As mentioned above, the detection sensitivity of the new matrices is expected to be lower (especially for opaque matrices) than for the current plates using the current detection system hardware. In addition to the use of transparent matrices, a number of hardware enhancements that could significantly improve sensitivity including: Higher sensitivity cooled CCD camera; Laser based illumination or other higher power density light source; and Faster (possibly non-telecentric) imaging optics.

In order to fully take advantage of the throughput afforded by 1,000,000 well plates, a large number of unique clones must be generated. Two alternative methods for preparing large numbers (107 to 109) of clones per day for screening can be used with the 100,000-well plates. They will both be tested for use with the 1,000,000-well density matrices and are described below. One effort will use Resorufin β-D-galactopyranoside (Molecular Probes #R-1159) as the fluorescent substrate and a positive β-galactosidase control clone (535-GL2) for both assay development and feasibility screening. This substrate and positive clone were well characterized and validated during the development of the 100,000-well platform.

Method 1: Screening Lambda Phage Libraries for Enzymatic Activity

Gene libraries cloned into lambda-based vectors are first titered by plating dilutions on soft agar in the presence of an appropriate E. coli host strain according to standard techniques. Using this titer information, an adequate amount of the lambda library is allowed to adsorb to the host. After 15 minutes, a mixture of growth medium and fluorescent substrate is then added to produce a final suspension having the following characteristics: [1] a density of host cells that will allow both sufficient growth and an effective multiplicity of infection, [2] an optimal concentration of fluorescent substrate for detection of the enzymatic activity, and [3] a density of phage particles such that, when loaded into a 1,000,000-well density matrix, each well will contain an average of 1-4 library clones. (Densities of 5-10 clones per well will be attempted once the initial details are worked out.) A sample of this suspension is plated on soft agar to determine the average seed density of library clones (concomitant titer). The remainder of the suspension is used to load the wells of the matrices. The plates are incubated at 37° C. for 16-24 hours (protected from light and evaporative loss; see note on Incubation below) to allow lytic multiplication of bacteriophage in the wells prior to detection and recovery.

Method 2: Screening Phagemid and Other Colony-Based Libraries for Enzymatic Activity

Phagemid libraries are produced from parental bacteriophage libraries using an in vivo excision process (Short et al., 1988). Following initial titering, these libraries are used to infect an appropriate E. coli host strain. After the 15-minute adsorption period, cells are supplied with a small amount of medium and allowed to grow at 30 degrees Celsuis without antibiotic selection for 45 minutes to allow expression of the antibiotic resistance gene present on the phagemid. The suspension is then plated onto solid plates containing antibiotic and allowed to grow at 30 degrees Celsius overnight. Amplified clones from the resulting antibiotic-resistant colonies are collected into a pooled suspension. A mixture of antibiotic, fluorescent substrate and growth medium is then added to produce the final suspension used to load the high-density matrices (with characteristics analogous to [2] and [3] above). A sample of this suspension is also plated onto solid agar plates containing antibiotic to determine the average seed density of library clones (concomitant titer). The matrices are then incubated at 30-37 degrees C. for 1-2 days (protected from light and evaporative loss; see note on Incubation below) to allow phagemid-containing host cells to multiply within the wells prior to detection and recovery.

Libraries created in other vectors (e.g. cosmid, fosmid, PAC, YAC, BAC, etc.) are also screened using this platform. Factors such as growth requirements, transformation modality, and transformation efficiency have to be taken into consideration when adapting a particular library vector to this technology. The use of a variety of library and vector types permits screening for small molecules and protein therapeutics in addition to novel enzymes.

The array plates are typically incubated in a humidified incubator at 90% relative humidity for 24 to 48 hours. The plates are stackable and designed such that each plate is contained within a humidity and temperature stable environment by the plates above and below it. Lids or extra plates filled with water are used at the top and bottom of each stack to seal the end plates. The incubation process requires validation of cell growth, evaporation, and condensation.

The growth of E. coli, which will be used as the enzyme screening host, has been clearly demonstrated in the 100,000 well array plate. Other types of cells including streptomyces, mammalian (Jurkat human leukemic T cells), and lambda phage have also been shown to grow in this format.

Cell growth in the 1,000,000-well density matrices will be verified by the same procedure used in for the 100,000-well plates. The number of colonies formed by plating the initial cell solution (diluted to 1 to 10 clones/well) will be compared to a culture of equal volume aspirated from the matrix after incubation. Although difficulties in cell growth are not anticipated, there are alternative strategies to mitigate these difficulties. The surface area to volume ratio of the 1,000,000-well density matrices is less favorable for oxygen diffusion into the assay solution than in the 100,000-well format. If oxygen diffusion appears to be limiting cell growth, we will evaluate methods for increasing oxygenation. Preliminary experiments have successfully demonstrated fluidic mixing in 200 μm diameter wells using paramagnetic beads in a fluctuating magnetic field and by agitation with sound pulses. Magnetic mixing has been shown to vastly improve the growth of Streptomyces in the 100,000-well format.

If necessary, these mixing methods could be employed to improve oxygen diffusion and cell growth. Other methods include oxygen saturation of the assay solution prior to plate filling, incubation in a high oxygen environment, and the addition of time-released oxygen generating compounds such as sodium percarbonate.

With a total assay volume of approximately 30 nl, controlling evaporation from the 1,000,000-well plates will be critical. However, as mentioned above, the surface to volume ratio is favorable for minimizing evaporation. Evaporation studies conducted in 100,000-well plates indicate a 10% loss of media volume over 24 hours. This loss is reduced to 5% with the addition of 10% glycerol. Because the surface area to volume ratio of the 1,000,000-well plates will be similar (if not more favorable) to the 100,000-well plates. Evaporation in the higher density matrices will be measured by filling the plates with typical assay media and weighing them at several time points over a 96-hour period. If stricter evaporation control is required, glycerol can be added.

The effects of condensation/moisture on the surface of the matrices are also considered. Because they are incubated in high-humidity environments, droplets on the outer surfaces of the matrices that remain after filling or condense during incubation may not evaporate and can cause well to well cross-contamination. These droplets can lead to the detection of false positives in wells neighboring a true positive as well as cause a blotchy appearance on the plate surface that obscures weak positives. Such problems with surface droplets remaining after filling the 100,000-well plates are avoided by letting them sit at room temperature until all of the surface moisture has evaporated. Avoiding condensation during incubation is accomplished by using strict temperature and humidity control. This issue is addressed by placing the filled plates in a programmable humidified chamber that starts with low humidity and increases it to the desired incubation humidity only after the plates have warmed to the chamber temperature. Once warm, the stacked plates form a relatively stable thermal mass immune to the small temperature fluctuations in the chamber. Surface moisture control issues will be similar in the higher density plates. The matrices will be tested to see if these methods successfully control surface moisture.

Negative libraries spiked with the positive β-gal clone at a defined frequency will be the first subjects of a feasibility screen. The same screen will be performed in parallel in a conventional microtiter format for comparison. Once this is proven, screening will proceed (again in parallel with microtiter format) to libraries known to contain positive clones. A mixed population library was validated for this purpose during the development of the 100,000-well platform and will be used for the 1,000,000-well feasibility screening. These experiments will be performed for both lambda-based and phagemid-based library screens since clonal amplification rates, and thus signal intensities, may differ between bacteriophage and whole cell assays.

Validation of the feasibility screens can be performed by simply comparing the number of positive wells in the fluorescence images of the 1,000,000-well matrices to those in a 100,000-well array plate filled with the identical assay solution.

Further verification will be done in standard microtiter format. The number of positive wells is a function of the concentration of positive clones in the initial assay solution and the volume of the wells. Since the well volume of the 1,000,000-well matrices is approximately 1/10th that of the 100,000 well plates, the expected number of positive wells should also be about 1/10th when loading the same initial assay solution.

The array of capillaries can be arranged to fit within a footprint of a microtiter plate, one standard of which is a footprint of 3.3″×5″. Within that footprint, up to 1,000,000 or more capillaries, or wells, can be provided in the array. A 1,000,000 well platform for screening gene libraries from mixed populations of organisms for novel enzymatic activities provides an ultra high-throughput screening platform in the 3.3″×5″ footprint of a standard microtiter plate. In this format each well includes a capillary having a diameter of 200 μm, and which holds 250 nl. The array platform permits rapid screening of genes and gene pathways, and increases the productivity of discovery and gene optimization programs for products such as novel enzymes, protein therapeutics, compounds and small molecule drugs. Any number of novel enzymes of various catalytic classes (e.g., amylases, proteases, secondary amidases) can be discovered using the array platform. The same proprietary cost effective process by which the 100,000-well plates are made can be utilized to make the 1,000,000-well plates for smaller, non-biological applications.

The array screening platform greatly expands the amount of molecular diversity that can be screened to discover new products. Using 1,000,000-well plates, employing over 12,000 wells per square centimeter, more than one billion clones per day can be screened using standard liquid phase fluorescent assays, while at the same time reducing equipment and operator time through massively parallel dispensing and reading of biological samples. Additionally, the 1,000,000-well plates, with wells each about half the diameter of a human hair, are be reusable and require only miniscule volumes of reagents, making them highly cost effective and environmentally responsible.

Increasing the liquid phase screening density from 100,000 to 1,000,000 wells per microtiter plate footprint represents a 10× increase in density that contributes to accelerated discovery and development of commercial products, such as antibody and protein therapeutic programs that require rapid screening of very large numbers of antibody and protein variants created by evolution technologies. This invention includes the design and fabrication of 1 cm square matrices with 1,000,000 well/plate density (i.e. 12,000 wells/cm2) using a process that is scalable to full microtiter plate sized arrays.

The platform can be utilized to develop a novel liquid phase nitrilase assay in the 1,000,000-well format, as well as screening gene libraries from mixed populations of organisms for chiral nitrilases for use in the manufacture of chemical intermediates for chiral therapeutic compounds.

Naked Biopanning involves the direct screening or enrichment for a gene or gene cluster from environmental genomic DNA. The enrichment for or isolation of the desired genomic DNA is performed prior to any cloning, gene-specific PCR or any other procedure that may introduce unwanted bias affecting downstream processing and applications due to toxicity or other issues. Several methodologies can be described for this type of sequence based discovery. These generally include the use of nucleic acid probe(s) that is(are) partially or completely homologous to the target sequence in conjunction with the binding of the probe-target complex to a solid phase support. The probe(s) may be polynucleotide or modified nucleic acid, such as peptide nucleic acid (PNA) and may be used with other facilitating elements such as proteins or additional nucleic acids in the capture of target DNA. An amplification step which does not introduce sequence bias may be used to ensure adequate yield for downstream applications.

An example of a Naked Biopanning approach can be found in the use of RecA protein and a complement-stabilized D-loop (csD-loop) structure (Jayasena & Johnston, 1993; Sena and Zarling, 1993) to target genomic DNA of interest. It does not involve complete denaturation of the target DNA and therefore is of particular interest when one is attempting to capture large genomic fragments. The following method incorporates the ClonCapture™ cDNA selection procedure (CLONTECH Laboratories, Inc.), with some modification, to take advantage of csD-loop formation, a stable structure which may be used to capture genomic DNA containing an internal target sequence:

Environmental genomic DNA is cleaved into fragments (fragment size depends upon type of target and desired downstream insert size if making a pre-enriched library) using mechanical shearing or restriction digest. Fragments are size selected according to desired length and purified. A biotinylated dsDNA probe is produced, based upon existing knowledge of conserved regions within the target, by PCR from a positive clone or by synthetic means. The probe can be internally (ex. incorporation of biotin 21-dCTP) or end labeled with biotin. It must be purified to remove any unincorporated biotin. The probe is heat denatured (5 min. at 95° C.) and placed immediately on ice. The denatured probe is then reacted with RecA and an ATP mix containing ATP and a nonhydrolyzable analog (15 min. at 37° C.). The target DNA is added and incubated with the RecA/biotinylated probe nucleofilaments to form the csD-loop structure (20 min. at 37° C.). The RecA is then removed by treatment with proteinase K and SDS. After inactivating the proteinase K with PMSF, washed and blocked (with sonicated salmon sperm DNA) streptavidin paramagnetic beads are transferred to the reaction and incubated to bind the csD-loop complex to the support (rotate 30 min. at room temp.). The unbound DNA is removed and may be saved for use as target for a different probe. The beads are thoroughly washed and the enriched population is eluted using an alkaline buffer and transferred off. The enriched DNA is then ethanol precipitated and is ready for ligation and pre-enriched library preparation.

Other stable complexes may be used instead of the RecA/csD-loop structure for the capture of genomic DNA. For instance, PNAs may be used, either as “openers” to allow insertion of a probe into dsDNA (Bukanov et al., 1998), or as tandem probes themselves (Lohse et al., 1999). In the first case, PNAs bind to two short tracts of homopurines that are in close proximity to each other. They form P-loop structures, which displace the unbound strand and make it available for binding by a probe, which can then be used to capture the target using an affinity capture method involving a solid phase. Likewise, PNAs may be used in a “double-duplex invasion” to form a stable complex and allow target recovery.

Simpler methods may be used in the retrieval of targets from environmental genomic DNA that involve complete denaturation of the DNA fragments. After cutting genomic DNA into fragments of the desired length via mechanical shearing or through the use of restriction enzymes, the target DNA may be bound to a solid phase using a direct hybridization affinity capture scheme. A nucleic acid probe is covalently bound to a solid phase such as a glass slide, paramagnetic bead, or any type of matrix in a column, and the denatured target DNA is allowed to hybridize to it. The unbound fraction may be collected and rehybridized to the same probe to ensure a more complete recovery, or to a host of different probes, as a part of a cascade scenario, where a population of environmental genomic DNA is subsequently panned for a number of different genes or gene clusters.

Linkers containing restriction sites and sites for common primers may be added to the ends of the genomic fragments using sticky-ended or blunt-ended ligations (depending upon the method used for cutting the genomic DNA). These enable one to amplify the size-selected inserted fragment population by PCR without significant sequence bias. Thus, after using any of the abovementioned techniques for isolation or enrichment, one may help to ensure adequate recovery for downstream processing. Furthermore, the recovered population is ready for cutting and ligation into a suitable vector as well as containing the priming sites for sequencing at any time.

A variation of the above scheme involves including a tag from a combinatorial synthesis of polynucleotide tags (Brenner et al., 1999) within the linker that is attached onto the ends of the genomic fragments. This allows each fragment within the starting population to have its own unique tag. Therefore, when amplified with common primers, each of these uniquely tagged fragments give rise to a multitude of in vitro clones which are then bound to the paramagnetic bead containing millions of copies of the complementary, covalently bound anti-tag. A fluorescently labeled, target specific probe may be subsequently hybridized to the target-containing beads. The beads may be sorted using FACS, where the positives may be sequenced directly from the beads and the insert may be cut out and ligated into the desired vector for further processing. The negative population may be hybridized with other probes and resorted as part of the cascade scenario previously described.

Transposon technology may allow the insertion of environmental genomic DNA into a host genome through the use of transposomes (Goryshin & Reznikoff, 1998) to avoid bias resulting from expression of toxic genes. The host cells are then cultured to provide more copies of target DNA for discovery, isolation, and downstream processes.

Without further elaboration, it is believed that one skilled in the art can, using the preceding description, utilize the present invention to its fullest extent. The following examples are to be considered illustrative and thus are not limiting of the remainder of the disclosure in any way whatsoever.

### EXAMPLE 1

### DNA Isolation and Library Construction

The following outlines the procedures used to generate a gene library from a mixed population of organisms.

DNA Isolation.

DNA is isolated using the IsoQuick Procedure as per manufacturer's instructions (Orca, Research Inc., Bothell, Wash.). DNA can be normalized according to Example 2 below. Upon isolation the DNA is sheared by pushing and pulling the DNA through a 25G double-hub needle and a 1-cc syringes about 500 times. A small amount is run on a 0.8% agarose gel to make sure the majority of the DNA is in the desired size range (about 3-6 kb).

Blunt-Ending DNA.

The DNA is blunt-ended by mixing 45 ul of 10× Mung Bean Buffer, 2.0 ul Mung Bean Nuclease (150 u/ul) and water to a final volume of 405 ul. The mixture is incubate at 37° C. for 15 minutes. The mixture is phenol/chloroform extracted followed by an additional chloroform extraction. One ml of ice cold ethanol is added to the final extract to precipitate the DNA. The DNA is precipitated for 10 minutes on ice. The DNA is removed by centrifugation in a microcentrifuge for 30 minutes. The pellet is washed with 1 ml of 70% ethanol and repelleted in the microcentrifuge. Following centrifugation the DNA is dried and gently resuspended in 26 ul of TE buffer.

Methylation of DNA.

The DNA is methylated by mixing 4 ul of 10× EcoR I Methylase Buffer, 0.5 ul SAM (32 mM), 5.0 ul EcoR I Methylase (40 u/ul) and incubating at 37° C., 1 hour. In order to insure blunt ends, add to the methylation reaction: 5.0 ul of 100 mM MgCl₂, 8.0 ul of dNTP mix (2.5 mM of each dGTP, dATP, dTTP, dCTP), 4.0 ul of Klenow (5 u/ul) and incubate at 12° C. for 30 minutes.

After 30 Minutes Add 450 ul 1× STE.

The mixture is phenol/chloroform extracted once followed by an additional chloroform extraction. One ml of ice cold ethanol is added to the final extract to precipitate the DNA. The DNA is precipitated for 10 minutes on ice. The DNA is removed by centrifugation in a microcentrifuge for 30 minutes. The pellet is washed with 1 ml of 70% ethanol, repelleted in the microcentrifuge and allowed to dry for 10 minutes.

Ligation.

The DNA is ligated by gently resuspending the DNA in 8 ul EcoR I adaptors (from Stratagene's cDNA Synthesis Kit), 1.0 ul of 10× Ligation Buffer, 1.0 ul of 10 mM rATP, 1.0 ul of T4 DNA Ligase (4Wu/ul) and incubating at 4° C. for 2 days. The ligation reaction is terminated by heating for 30 minutes at 70° C.

Phosphorylation of Adaptors.

The adaptor ends are phosphorylated by mixing the ligation reaction with 1.0 ul of 10× Ligation Buffer, 2.0 ul of 10 mM rATP, 6.0 ul of H₂O, 1.0 ul of polynucleotide kinase (PNK) and incubating at 37° C. for 30 minutes. After 30 minutes 31 ul H₂O and 5 ml 10× STE are added to the reaction and the sample is size fractionate on a Sephacryl S-500 spin column. The pooled fractions (1-3) are phenol/chloroform extracted once followed by an additional chloroform extraction. The DNA is precipitated by the addition of ice cold ethanol on ice for 10 minutes. The precipitate is pelleted by centrifugation in a microfuge at high speed for 30 minutes. The resulting pellet is washed with 1 ml 70% ethanol, repelleted by centrifugation and allowed to dry for 10 minutes. The sample is resuspended in 10.5 ul TE buffer. Do not plate. Instead, ligate directly to lambda arms as above except use 2.5 ul of DNA and no water.

Sucrose Gradient (2.2 ml) Size Fractionation.

Stop ligation by heating the sample to 65° C. for 10 minutes. Gently load sample on 2.2 ml sucrose gradient and centrifuge in mini-ultracentrifuge at 45K, 20° C. for 4 hours (no brake). Collect fractions by puncturing the bottom of the gradient tube with a 20G needle and allowing the sucrose to flow through the needle. Collect the first 20 drops in a Falcon 2059 tube then collect 10 1-drop fractions (labeled 1-10). Each drop is about 60 ul in volume. Run 5 ul of each fraction on a 0.8% agarose gel to check the size. Pool fractions 1-4 (about 10-1.5 kb) and, in a separate tube, pool fractions 5-7 (about 5-0.5 kb). Add 1 ml ice cold ethanol to precipitate and place on ice for 10 minutes. Pellet the precipitate by centrifugation in a microfuge at high speed for 30 minutes. Wash the pellets by resuspending them in 1 ml 70% ethanol and repelleting them by centrifugation in a microfuge at high speed for 10 minutes and dry. Resuspend each pellet in 10 ul of TE buffer.

Test Ligation to Lambda Arms.

Plate assay by spotting 0.5 ul of the sample on agarose containing ethidium bromide along with standards (DNA samples of known concentration) to get an approximate concentration. View the samples using UV light and estimate concentration compared to the standards. Fraction 1-4=>1.0 ug/ul. Fraction 5-7=500 ng/ul.

Prepare the following ligation reactions (5 μl reactions) and incubate 4° C., overnight:

| | | | | Lambda | | T4 DNA |
|---|---|---|---|---|---|---|
| | | 10× Ligase | 10 mM | arms | Insert | Ligase (4 |
| Sample | H₂O | Buffer | rATP | (ZAP) | DNA | Wu/(l) |
| Fraction 1-4 | 0.5 ul | 0.5 ul | 0.5 ul | 1.0 ul | 2.0 ul | 0.5 ul |
| Fraction 5-7 | 0.5 ul | 0.5 ul | 0.5 ul | 1.0 ul | 2.0 ul | 0.5 ul |

Test Package and Plate.

Package the ligation reactions following manufacturer's protocol. Stop packaging reactions with 500 ul SM buffer and pool packaging that came from the same ligation. Titer 1.0 ul of each pooled reaction on appropriate host (OD₆₀₀=1.0) [XLI-Blue MRF]. Add 200 ul host (in mM MgSO₄) to Falcon 2059 tubes, inoculate with 1 ul packaged phage and incubate at 37° C. for 15 minutes. Add about 3 ml 48° C. top agar [50 ml stock containing 150 ul IPTG (0.5M) and 300 ul X-GAL (350 mg/ml)] and plate on 100 mm plates. Incubate the plates at 37° C., overnight.

Amplification of Libraries (5.0×10⁵ Recombinants From Each Library).

Add 3.0 ml host cells (OD₆₀₀=1.0) to two 50 ml conical tube and inoculate with 2.5×10⁵ pfu of phage per conical tube. Incubate at 37° C. for 20 minutes. Add top agar to each tube to a final volume of 45 ml. Plate each tube across five 150 mm plates. Incubate the plates at 37° C. for 6-8 hours or until plaques are about pin-head in size. Overlay the plates with 8-10 ml SM Buffer and place at 4° C. overnight (with gentle rocking if possible).

Harvest Phage.

Recover phage suspension by pouring the SM buffer off each plate into a 50-ml conical tube. Add 3 ml of chloroform, shake vigorously and incubate at room temperature for 15 minutes. Centrifuge the tubes at 2K rpm for 10 minutes to remove cell debris. Pour supernatant into a sterile flask, add 500 ul chloroform and store at 4° C.

Titer Amplified Library.

Make serial dilutions of the harvested phage (for example, 10⁻⁵=1 ul amplified phage in 1 ml SM Buffer; 10⁻⁶=1 ul of the 10⁻³ dilution in 1 ml SM Buffer). Add 200 ul host (in 10 mM MgSO₄) to two tubes. Inoculate one tube with 10 ul 10⁻⁶ dilution (10⁻⁵). Inoculate the other tube with 1 ul 10⁻⁶ dilution (10⁻⁶). Incubate at 37° C. for 15 minutes. Add about 3 ml 48° C. top agar [50 ml stock containing 150 ul IPTG (0.5M) and 375 ul X-GAL (350 mg/ml)] to each tube and plate on 100 mm plates. Incubate the plates at 37° C., overnight. Excise the ZAP II library to create the pBLUESCRIPT library according to manufacturers protocols (Stratagene).

### EXAMPLE 2

### Construction of a Stable, Large Insert Picoplankton Genomic DNA Library

Cell collection and preparation of DNA. Agarose plugs containing concentrated picoplankton cells were prepared from samples collected on an oceanographic cruise from Newport, Oreg. to Honolulu, Hi. Seawater (30 liters) was collected in Niskin bottles, screened through 10 m Nitex, and concentrated by hollow fiber filtration (Amicon DC10) through 30,000 MW cutoff polyfulfone filters. The concentrated bacterioplankton cells were collected on a 0.22 m, 47 mm Durapore filter, and resuspended in 1 ml of 2× STE buffer (1M NaCl,0.1M EDTA, 10 mM Tris, pH 8.0) to a final density of approximately 1×10¹⁰ cells per ml. The cell suspension was mixed with one volume of 1% molten Seaplaque LMP agarose (FMC) cooled to 40 C, and then immediately drawn into a 1 ml syringe. The syringe was sealed with parafilm and placed on ice for 10 min. The cell-containing agarose plug was extruded into 10 ml of Lyses Buffer (10 mM Tris pH 8.0, 50 mM NaCl, 0.1 M EDTA, 1% Sarkosyl, 0.2% sodium deoxycholate, 1 mg/ml lysozyme) and incubated at 37 C for one hour. The agarose plug was then transferred to 40 mls of ESP Buffer (1% Sarkosyl, 1 mg/ml proteinase K, in 0.5M EDTA), and incubated at 55 C for 16 hours. The solution was decanted and replaced with fresh ESP Buffer, and incubated at 55 C for an additional hour. The agarose plugs were then placed in 50 mM EDTA and stored at 4 C shipboard for the duration of the oceanographic cruise.

One slice of an agarose plug (72 1) prepared from a sample collected off the Oregon coast was dialyzed overnight at 4 C against 1 mL of buffer A (100 mM NaCl, 10 mM Bus Tris Propane-HCl, 100 g/ml acetylated BSA: pH 7.0@25 C) in a 2 mL microcentrifuge tube. The solution was replaced with 250 1 of fresh buffer A containing 10 mM MgCl, and 1 mh4 DTT and incubated on a rocking platform for 1 hr at room temperature. The solution was then changed to 250 l of the same buffer containing 4U of Sau3Al (NEB), equilibrated to 37 C in a water bath, and then incubated on a rocking platform in a 37 C incubator for 45 min. The plug was transferred to a 1.5 ml microcentrifuge tube and incubated at 68 C for 30 min to inactivate the enzyme and to melt the agarose. The agarose was digested and the DNA dephosphorylased using Gelase and HK-phosphatase (Epicentre), respectively, according to the manufacturer's recommendations. Protein was removed by gentle phenol/chloroform extraction and the DNA was ethanol precipitated, pelleted, and then washed with 70% ethanol. This partially digested DNA was resuspended in sterile H,O to a concentration of 2.5 ng/l for ligation to the pFOSl vector.

PCR amplification results from several of the agarose plugs (data not shown) indicated the presence of significant amounts of archaeal DNA. Quantitative hybridization experiments using rRNA extracted from one sample, collected at 200 m of depth off the Oregon Coast, indicated that planktonic archaea in this assemblage comprised approximately 4.7% of the total picoplankton biomass. This sample corresponds to “PAC1 ”-200 m in Table 1 of DeLong et al. (DeLong, 1994), which is incorporated herein by reference. Results from archaeal-biased rDNA PCR amplification performed on agarose plug lysates confirmed the presence of relatively large amounts of archaeal DNA in this sample. Agarose plugs prepared from this picoplankton sample were chosen for subsequent fosmid library preparation. Each 1 ml agarose plug from this site contained approximately 7.5×10⁵ cells, therefore approximately 5.4×10⁵ cells were present in the 72 l slice used in the preparation of the partially digested DNA.

Vector arms were prepared from pFOSl as described by Kim et al. (Kim, 1992). Briefly, the plasmid was completely digested with AstII, dephosphorylated with HK phosphatase, and then digested with BamHI to generate two arms, each of which contained a cos site in the proper orientation for cloning and packaging ligated DNA between 35-45 kbp. The partially digested picoplankton DNA was ligated overnight to the PFOS 1 arms in a 15 l ligation reaction containing 25 ng each of vector and insert and 1U of T4 DNA ligase (Boehringer-Mannheim). The ligated DNA in four microliters of this reaction was in vitro packaged using the Gigapack XL packaging system (Stratagene), the fosmid particles transfected to E. coli strain DH10B (BRL), and the cells spread onto LB_{cm15} plates. The resultant fosmid clones were picked into 96-well microliter dishes containing LB_{cm15} supplemented with 7% glycerol. Recombinant fosmids, each containing ca. 40 kb of picoplankton DNA insert, yielded a library of 3.552 fosmid clones, containing approximately 1.4×10⁸ base pairs of cloned DNA. All of the clones examined contained inserts ranging from 38 to 42 kbp. This library was stored frozen at −80 C for later analysis.

Numerous modifications and variations of the present invention are possible in light of the above teachings; therefore, within the scope of the claims, the invention may be practiced other than as particularly described.

### EXAMPLE 3

### CsCl-Bisbenzimide Gradients

Gradient visualization by UV:

Visualize gradient by using the UV handlamp in the dark room and mark bandings of the standard which will show the upper and lower limit of GC-contents.

Harvesting of the gradients:

1. Connect Pharmacia-pump LKB P1 with fraction collector (BIO-RAD model 2128).

2. Set program: rack 3, 5 drops (about 100 ul), all samples.

3. Use 3 microtiter-dishes (Costar, 96 well cell culture cluster).

4. Push yellow needle into bottom of the centrifuge tube.

5. Start program and collect gradient. Don't collect first and last 1-2 ml depending on where your markers are.

Dialysis

1. Follow microdialyzer instruction manual and use Spectra/Por CE Membrane MWCO 25,000 (wash membrane with ddH20 before usage).

2. Transfer samples from the microtiterdish into microdialyzer (Spectra/Por,

3. MicroDialyzer) with multipipette. (Fill dialyzer completely with TE, get rid of any air bubble, transfer samples very fast to avoid new air-bubbles).

4. Dialyze against TE for 1 hr on a plate stirrer.

DNA Estimation with PICOGREEN

1. Transfer samples (volume after dialysis should be increased 1.5-2 times) with multipipette back into microtiterdish.

2. Transfer 100 ul of the sample into Polytektronix plates.

3. Add 100 ul Picogreen-solution (5 ul Picogreen-stock-solution+995 ul TE buffer) to each sample.

4. Use WPR-plate-reader.

5. Estimate DNA concentration.

### EXAMPLE 4

### Bis-Benzimide Separation of Genomic DNA

A sample composed of genomic DNA from Clostridium perfringens (27% G+C), Escherichia coli (49% WC) and Micrococcus lysodictium (72% G+C) was purified on a cesium-chloride gradient. The cesium chloride (Rf=1.3980) solution was filtered through a 0.2 m filter and 15 ml were loaded into a 35 ml OptiSeal tube (Beckman). The DNA was added and thoroughly mixed. Ten micrograms of bis-benzimide (Sigma; Hoechst 33258) were added and mixed thoroughly. The tube was then filled with the filtered cesium chloride solution and spun in a VTi50 rotor in a Beckman L8-70 Ultracentrifuge at 33,000 rpm for 72 hours. Following centrifugation, a syringe pump and fractionator (Brandel Model 186) were used to drive the gradient through an ISCO UA-5 UV absorbance detector set to 280 nm. Three peaks representing the DNA from the three organisms were obtained. PCR amplification of DNA encoding rRNA from a 10-fold dilution of the E. coli peak was performed with the following primers to amplify eubacterial sequences:

Forward primer: (27F)
5-AGAGTTTGATCCTGGCTCAG-3
Reverse primer: (1492R)
5-GGTTACCTTGTTACGACTT-3

### EXAMPLE 5

### FACS/Biopanning

Infection of library lysates into Exp503 E.coli strain. 25 ml LB+Tet culture of Exp503 were cultured overnight at 37 C. The next day the culture was centrifuged at 4000 rpm for 10 minutes and the supernatant decanted. 20 ml 10 mM MgSO₄ was added and the OD₆₀₀ checked. Dilute to OD 1.0.

In order to obtain a good representation of the library, at least 2-fold (and preferably 5-fold) of the library lysate titer was used. For example: Titer of library lysate is 2×10⁶ cfu/ml. Need to plate at least 4×10⁶ cfu. Can plate approx. 500,000 microcolonies/150 mm LB-Kan plate. Need 8 plates. Can plate 1 ml of reaction/plate-need 8 mls of cells+lysate.

2-fold (ex. 2 ml) of library lysate was mixed with appropriate amount (e.g., 6 ml) of OD 1.0 Exp503. The sample was incubated at 37° C. for at least 1 hour. Plated 1 ml reaction on 150 mm LB-Kan plate×8 plates and incubated overnight at 30° C.

Harvesting, Induction, and Fixing of Library in Exp503 Cells.

Scrape all cells from plates into 20 ml LB using a rubber policeman. Dilute cells approx. 1:100 (200 ul cells/20 ml LB) and incubate at 37° C. until culture is OD 0.3. Add 1:50 dilution of 20% sterile Glucose and incubate at 37° C. until culture is OD 1.0. Add 1:100 dilution of 1 M MgSO₄. Transfer 5 ml of culture to a fresh tube and the remaining culture can be used as an uninduced control if desired or discarded. Add MOI 5 of CE6 bacteriophage to the remaining 5 ml of culture. (CE6 codes for T7 RNA Polymerase) (e.g., OD 1=8×10⁸ cells/ml×5 ml=4×10⁹ cells×MOI 5=2×10¹⁰ bacteriophage needed). Incubate culture+CE6 for 2 hr at 37° C. Cool on ice and centrifuge cells at 4000 rpm for 10 min. Wash with 10 ml PBS. Fix cells in 600 ul PBS+1.8 ml fresh, filtered 4% paraformaldehyde. Incubate on ice for 2 hrs. (4% Paraformaldehyde: Heat 8.25 ml PBS in flask at 65° C. Add 100 ul 1M NaOH and 0.5 g paraformaldehyde (stored at 4° C.) Mix until dissolved. Add 4.15 ml PBS. Cool to 0° C. Adjust pH to 7.2 with 0.5 M NaH₂PO₄. Cool to 0° C. Syringe filter. Use within 24 hrs). After fixing, centrifuge at 4000 rpm for 10 min. Resuspend in 1.8 ml PBS and 200 ul 0.1% NP40. Store at 4° C. overnight.

Hybridization of Fixed Cells.

Centrifuge fixed cells at 4000 rpm for 10 min. Resuspend in 1 ml 40 mM Tris pH7.6/0.2% NP40. Transfer 100 ul fixed cells to an eppendorf tube. Centrifuge for 1 min and remove supernatant. Resuspend each reaction in 50 ul Hybridization buffer (0.9 M NaCl; 20 mM Tris pH7.4; 0.01% SDS; 25% formamide—can be made in advance and stored at −20° C.). Add 0.5 nmol fluorescein-labeled primer to the appropriate reactions. Incubate with rocking at 46° C. for 2 hr. (Hybridization temperature may depend on sequence of primer and template.) Add 1 ml wash buffer to each reaction, rinse briefly and centrifuge for 1 min. Discard supernatant. (Wash buffer: 0.9 M NaCl; 20 mM Tris pH 7.4; 0.01% SDS). Add another 1 ml of wash buffer to each reaction, and incubate at 48° C. with rocking for 30 min. Centrifuge and remove supernatant. Visualize cells under microscope using WIB filter.

FACS Sorting.

Dilute cells in 1 ml PBS. If cells are clumping, sonicate for 20 seconds at 1.5 power. FAC sort the most highly fluorescent single-cells and collect in 0.5 ml PCR strip tubes (approximately one 96-well plate/library). PCR single-cells with vector specific primers to amplify the insert in each cell. Electrophores all samples on an agarose gel and select samples with single inserts. These can be re-amplified with Biotin-labeled primers, hybridized to insert-specific primers, and examined in an ELISA assay. Positive clones can then be sequenced. Alternatively, the selected samples can be re-amplified with various combinations of insert-specific primers, or sequenced directly.

### EXAMPLE 6

### Large Insert FACS Biopanning Protocol

1. Encapsulate 1 vial of 3% home-made SeaPlaque gel. Each vial of gel can make 10⁶ GMD. Take 100 ul melt frozen fosmid pMF21/DH10B library, OD600=0.4 to encapsulate, centrifuge down to 10 ul. Melt agarose gel, add 100 ul FBS (fetal bovine serum) and vortex. Place in 50 C water in a beaker. Add 10 ul culture, vortex and add to 17 ml mineral oil. Shake for about 30 times, place on the One Cell machine. Blend at 2600 rpm 1 min at room temperature and 2600 rpm 9 minutes on ice. Wash with PBS twice. Resuspend in 10 ml LB+Apr⁵⁰, shake at 37° C. for 4 hours at 230 rpm. Check microscopically to see the growth and size of microcolonies.

2. Centrifuge at 1500 rpm for 6 min. GMDs are resuspend in 5 ml of 2× SSC and can be saved at 4° C. for several days. Take 200 ul GMD in 2× SSC for each reaction.

3. Resuspend in 10 ml 2× SSC/5% SDS. Incubate 10 min at RT shaking or rotating. Centrifuge.

4. Resuspend in 5 ml lysis solution containing proteinase K. Incubate 30 min at 37° C. shaking or rotating. Centrifuge.

| Lysis Solution: | |
|---|---|
| 50 mM Tris pH8 | 0.75 ml 1M Tris |
| 50 mM EDTA | 1.5 ml 0.5M EDTA |
| 100 mM NaCl | 300 ul 5M NaCl |
| 1% Sarkosyl | 0.75 ml 20% Sarkosyl |
| 250 ug/ml Proteinase K | 375 ul proteinase K stock (10 mg/ml) |
| | 11.325 ml dH2O |

5. Resuspend in 5 ml denaturing solution. Incubate 30 min at RT shaking or rotating. Centrifuge at 1500 rpm for 5 min.

Denaturing Solution:

0.5M NaOH/1.5M NaCl

6. Resuspend in 5 ml neutralizing solution. Incubate 30 min at RT shaking or rotating. Centrifuge.

Neutralizing Solution:

0.5M Tris pH8/1.5M NaCl

7. Wash in 2× SSC briefly.

8. Aliquot 200 ul/RxN into microcentrifuge tubes, microcentrifuge and take out the 2× SSC. Add 130 ul “DIG EASY HYB” to prehyb for 45 minutes at 37° C. Do prehyb and hyb in Personal Hyb Oven.

9. Aliquot oligo probe and denature at 85° C. for 5 minutes, place on ice immediately. Add appropriate amount of probe (0.5-1 nmol/RXN) and return to rotating hyb. oven for O/N.

10. Prepare a 1% (10 mg/ml) solution of Blocking Reagent in PBS. Store at 4° C. for the day use.

11. Wash GMD's with 0.8 ml of 2× SSC/0.1%SDS RT 15 min, rotating. At the meantime, prewarm next wash solution.

12. Wash GMD's with 0.8 ml of 0.5× SSC/0.1%SDS 2×15 min at appropriate temp, rotating. If more stringency is required, the 2^{nd} wash can be done in 0.1× SSC/0.1%SDS.

13. Wash with 0.8 ml/RXN 2× SSC briefly.

14. Block the reaction w/130 ul 1% Blocking Reagent in PBS at RT for 30 minutes.

15. Add 1.4 ul anti-DIG-POD (so 1:100) and incubate at RT for 3 hours.

16. Wash GMDs w/0.8 ml PBS/RN 3×7 minutes at 37° C.

17. Prepare a tyramide working solution by diluting the tyramide stock solution 1:85 in Amplification buffer/0.0015% H2O₂. Apply 130 ul tyramide working solution at RT and incubate in the dark at RT for 30 minutes.

18. Wash 3× for 7 min. in 0.8 ml PBS buffer@37° C.

19. Visualize by microscope and FACS sort.

### EXAMPLE 7

### Biopanning Protocol

Preparing Insert DNA from the Lambda DNA

PCR amplify inserts using vector specific primers CA98 and CA103.

| | |
|---|---|
| CA98: | ACTTCCGGCTCGTATATTGTGTGG |
| CA103: | ACGACTCACTATAGGGCGAATTGGG |

These primers match perfectly to lambda ZAP Express clones (pBKCMV).

Reagents: Lambda DNA prepared from the libraries to be panned (Librarians)

Roche Expand Long Template PCR System #1-759-060

Pharmacia dNTP mix #27-2094-01 or

Roche PCR Nucleotide Mix (10 mM) #1-581-295 or

Roche dNTP's—PCR grade #1-969-064

1. Make the insert amplification mix:

X μl dH₂O (final 50 μl)

5 μl 10× Expand Buffer #2 (22.5 mM MgCl₂)

0.5 or 0.625 μl dNTP mix (20 mM each dNTP)

10 ng (approx) lambda DNA per library (usually 1 μl or 1 μl 1:10 diln)

1-2 μl CA98 (100 ng/μl or 15 μM)

1-2 μl CA103 (100 ng/μl or 15 μM)

0.5 μl Expand Long polymerase mix

2. PCR amplify:

Robocycler

| | | |
|---|---|---|
| 95° C. | 3 minute | × 1 cycle |
| 95° C. | 1 minute | |
| 65° C. | 45 seconds | × 30 cycles |
| 68° C. | 8 minute | |
| 68° C. | 8 minute | × 1 cycle |
| 6° C. | | ∞ |

3. Analyze 5 μl of reaction product on a gel.

Note: The reaction product should be a strong smear of products usually ranging from 0.5-5 kb in size and centered around 1.5-2 kb.

Prepare Biotinylated Hook

Reagents: PCR reagents

Biotin-14-dCTP (BRL #19518-018)

Individual dNTP stock solutions (Roche dNTP's #1-969-064)

Gene specific template and primers

PCR purification kit (Roche #1732668 or Qiagen Qiaquick #28106)

1. Make 10× biotin dNTP mix:

150 μl biotin-14-dCTP

3 μl 100 mM dATP

3 μl 100 mM dGTP

3 μl 100 mM dTTP

1.5 μl 100 mM dCTP

2. Make PCR mix:

74 μl water

10 μl 10× Expand Buffer #1

10 μl 10 × biotin dNTP mix (step #1)

2 μl Primer #1 (100 ng/μl)

2 μl Primer #2 (100 ng/μl)

1 μl template (gene specific) (100 ng/μl)

1 μl Expand Long polymerase mix

3. PCR amplify:

Robocycler

| | | |
|---|---|---|
| 95° C. | 3 minute | × 1 cycle |
| 95° C. | 45 seconds | |
| * ° C. | 45 seconds | × 30 cycles |
| 68° C. | ** minute | |
| 68° C. | 8 minute | × 1 cycle |
| 6° C. | | ∞ |

* Use an annealing temperature appropriate for your primers.
** Allow 1 minute/kb of target length.

4. Clean up the reaction product using a PCR purification kit. Elute in 50 μl ST.1E or Qiagen's EB buffer (10 mM Tris pH 8.5).

5. Check 5 μl on an agarose gel.

Note: The product may be slightly larger than expected due to the incorporation of biotin.

Biopanning

Reagents: Streptavidin-conjugated paramagnetic beads (CPG MPG-Streptavidin

10 mg/ml #MSTR0502)(Dynal Dynabeads M-280 Streptavidin)

Sonicated, denatured salmon sperm DNA (heated to 95° C., 5 min) (Stratagene #201190)

PCR reagents

dNTP mix

Magnetic particle separator

Topo-TA cloning kit with Top10F′ comp cells (Invitrogen #K4550-40)

High Salt Buffer: 5M NaCl, 10 mM EDTA, 10 mM Tris pH 7.3

1. Make the following reaction mix for each library/hook combination:

5 μg insert DNA (PCR amplified lambda DNA)

100 ng Biotinylated hook (100 ng total if using more than one hook)

4.5 μl 20× SSC for a 3× final concentration (or High Salt buffer)

X μl dH₂O for a final volume of 30 μl

2. Denature by heating to 95° C. for 10 min. (Robocycler works well for this step).

3. Hybridize at 70° C. for 90 min. (Robocycler)

4. Prepare 100 μl of MPG beads for each sample:

Wash 100 μl beads two times with 1 ml 3× SSC

Resuspend in: 50 μl 3× SSC (or High Salt buffer)

10 μl Sonicated, denatured salmon sperm DNA (10 mg/ml) to block (or 100 ng total)

(Do not ice)

5. Add the hybridized DNA to the washed and blocked beads.

6. Incubate at room temp for 30 min, agitating gently in the hybridization oven.

7. Wash twice at room temp with 1 ml 0.1× SSC/0.1% SDS, (or high salt buffer) using magnetic particle separator.

8. Wash twice at 42° C. with 1 ml 0.1× SSC/0.1% SDS (or high salt buffer) for 10 min each. (magnet)

9. Wash once at room temp with 1 ml 3× SSC. (magnet)

10. Elute DNA by resuspending the beads in 50 μl dH₂O and heating the beads to 70° C. for 30 min or 85° C. for 10 min. in the hyb oven (or thermomixer at 500 rpm). Separate using magnet, and discard the beads.

11. PCR amplify 1-5 μl of the panned DNA using the same protocol as Preparing Insert DNA from the Lambda DNA above.

12. Check 5 μl on agarose gel.

Note: The reaction product should be a strong smear of products usually ranging from 0.5-5 kb in size and centered around 1.5-2 kb.

13. Clone 1-4 μl into pCR2.1-TopoTA cloning vector.

14. Transform 2×3 μl into Top10F′ chemically comp cells. Plate each transformation on 2×150 mm LB-kan plates. Incubate at 30° C. overnight.

(Ideal density is ˜3000 colonies per plate).

Repeat transformation if necessary to get a representative number of colonies per library. Archive the Biopanned DNA.

15. Transfer plates to Hybridization group, along with appropriate templates and a single primer for run off PCR ³²P-labeling reactions.

Analysis of Results

1. Filter lifts from plates will be performed, and hybridized to the appropriate probe. Resultant films will be given to the Biopanned.

2. Align films to original colony plates. Colonies corresponding to positive “dots-on-film” should be toothpicked, patched onto an LB-Kan plate, and inoculated in 4 ml TB-Kan. For automation, inoculate 1 ml TB-kan in a 96-well plate and incubate 18 hrs. at 37° C.

3. Overnight cultures are mini-prepped (Biomek if possible). Digest with EcoRI to determine insert size.

2 μl DNA

0.5 μl EcoRI

1 μl 10× EcoRI buffer

6.5 μl dH₂O

Incubate at 37° C. for 1 hr. Check insert size on agarose gel.

Large insert clones (>500 bp) are then PCR confirmed if possible with gene specific primers.

4. Putative positive clones are then sequenced.

5. Glycerol stocks should be made of all interesting clones (>500 bp).

### EXAMPLE 8

### High Throughput Cultivation of Marine Microbes from Sea Sample

17. Preparation of Cell Suspension

Cells were obtained after filtering 110 L of surface water through a 0.22 μm membrane. The cell pellet was then resuspended with seawater and a volume of 100 μL was used for cell encapsulation. This provided cell numbers of approximately 107 cells per mL.

18. Cell Encapsulation into GMDs

The following reagents were used: CelMix™ Emulsion Matrix and CelGel™ Encapsulation Matrix (One Cell Systems, Inc., Cambridge, Mass.), Pluronic F-68 solution and Dulbecco's Phosphate Buffered Saline (PBS, without Ca²⁺ and Mg²⁺). Scintillation vials each containing 15 ml of CelMix™ emulsion matrix were placed in a 40° C. water bath and were eliquilibrated to 40° C. for a minimum of 30 minutes. 30 ul of Pluronic Solution F-68 (10%) was added to each of 6 vials of melted CelGel™ agarose. The agarose mixture was incubated to 40° C. for a minimum of 3 minutes. 100 ul of cells (resuspended in PBS) were added per 6 vials of the CelGel™ bottles and the resulting mixture was incubated at 40° C. for 3 minutes. Using a 1 ml pipette and avoiding air bubbles, the CelGel™-cell mixture was added dropwise to the warmed CelMix™ in the scintillation vial. This mixture was then emulsified using the CellSys100™ MicroDrop maker as follows: 2200 rpm for 1 minute at room temperature (RT), then 2200 rpm for 1 minute on ice, then 1100 rpm for 6 minutes on ice, resulting in an encapsultion mixture comprised of microdrops that were approximately 10-20 microns in diameter. The encapsulation mixture was then divided into two 15 ml conical tubes and in each vial, the emulsion was overlayed with 5 ml of PBS. The vials tubes were then centrifuged at 1800 rpm in a bench top centrifuge for 10 minutes at RT, resulting in a visible Gel MicroDrop (GMD) pellet. The oil phase was then removed with a pipette and disposed of in an oil waste container. The remaining aqueous supernatant was aspirated and each pellet was resuspended in 2 ml of PBS. Each resuspended pellet was then overlayed with 10 ml of PBS. The GMD suspension was then centrifuged at 1500 rpm for 5 minutes at RT. Overlaying process is repeated and the GMD suspension is centrifuged again to remove all free-living bacteria. The supernatant was then removed and the pellet was resuspended in 1 ml of seawater. 10 ul of the GMD suspension was then examined under the microscope in order to check for uniform GMD size and containment of then encapsulated organism into the GMD. This protocol resulted in 1 to 4 cells encapsulated in each GMD.

19. Sorting of GMDs Containing Single Cells for Identification by 16S rRNA Gene Sequence

On the first day of cultivation we sorted occupied GMDs that contained one to 4 cells, although most had only single cells. The sorting was done in a Mo-Flo instrument (Cytomation) by staining the cells inside the GMDs with Syto9 and then selecting green fluorescence (from the stain) and side-scatter as parameters for sorting gates. The staining was necessary since the cells are much smaller than E. coli and therefore show very low light-scatter signals. The target GMDs were sorted into a 96-well plate containing a PCR mixture and ready to be amplified immediately after sorting. We used a Hotstart enzyme (Qiagen) such as no reaction would occur before boiling for 15 min and therefore allows to work at room temperature before amplification. Before starting the PCR it was necessary to radiate the PCR mixture with a Stratalinker (Stratagene) at full power for 14 min to cross-link any potential genomic DNA present in the mixture before sorting. The primers used include the pair 27F and 1392R and 27F and 1522R according to the positions in E. coli gene sequence. The primers were obtained from IDT-DNA Technologies and were purified by HPLC. The primer concentration used in the reactions was 0.2 μM. We used a “touchdown” program consisting of 3 stages: a) boiling 15 min, b) 15 cycles decreasing the annealing temperature from 62 to 55° C. by 0.5 degrees per cycle, c) a series of cycles (20-40) increasing the annealing time 1 sec per cycle starting with 30 sec but keeping the temperature constant at 55° C. All the other stages of the PCR were as recommended by manufacturer. This protocol allowed the amplification of the 16S rRNA gene from individual cells encapsulated or small consortia of cells. The PCR products were then cloned into TOPO-TA (Invitrogen) cloning vectors and sequenced by dye-termination cycle sequencing (Perkin-Elmer ABI).

Cell Growth of Encapsulated Cells Inside GMDs

The encapsulated GMDs were placed into chromatography columns that allowed the flow of culture media providing nutrients for growth and also washed out waste products from cells. The experiment consisted of 4 treatments including the use of seawater, and amendments (inorganic nutrients including trace metals and vitamins, amino acids including trace metals and vitamins, and diluted rich organic marine media). This different set of nutrients provided a gradient to bias different microbial populations. The seawater used as base for the media was filter sterilized through a 1000 kDa and a 0.22 μm filter membranes prior to amendment and introduction to the columns. The cells were then incubated for a period of 17 weeks and cell growth was monitored by phase contrast microscopy. Cell identification was done by 16S rRNA gene sequence of grown colonies.

20. Sorting of GMDs Containing Colonies Consisting of One or More Cell Types

To identify the diversity and the community composition of the different treatments we performed a “bulk sorting” of the GMDs. This was done by taking a subsample of the GMDs from each column and run them into the Flow-cytometer. We selected as gating criteria forward- and side-scatter as occupied GMDs with a colony of 10 or more cells of individual cell sizes ranging from 0.5 to 5 μm were easy to discriminate from empty GMDs. We verified each time by phase contrast microscopy that we selected the correct gate for sorting. We then sorted a total of 300 GMDs per each individual PCR reaction (prepared as above) and ran the reaction in a thermocycler for a total of 50 to 60 cycles to have enough PCR product to be visualized by gel electrophoresis. The resulting PCR reactions from the same column were combined (2 to 4 replicates), cloned and sequenced as above to assess the phylogenetic diversity from each column and observe the bias effect resulting from the use of different nutrient regimes.

Gene Sequencing and Phylogenetic Analyses

The gene sequences were aligned and compared to our 16S rRNA database with the ARB phylogenetic program. Maximum Parsimony and neighbor joining trees were constructed using the amplified gene sequences (approximately 1400 bp).

### EXAMPLE 9

### Microextraction Procedure

A single copy of Streptomyces containing clones from a mixed population are FACS-sorted onto agar, allowed to develop into individual colonies, and bioassayed as individual clones.

Construction of a Clone Expressing a Bioactive Metabolite

A genomic library of Streptomyces murayamaensis is constructed in pJO436 (Bierman et al., Gene 1991116:43-49) vector and hybridized with probes for polyketide synthase. A clone (1B) which hybridized was chosen and shuttled into Streptomyces venezuelae ATCC 10712 strain. The vector pMF17 was also introduced into S. diversa as a negative control. When bioassayed on solid media, clone 1B expressed strong bioactivity towards Micrococcus luteus suggesting that the insert present in clone 1B encoded a bioactive polyketide molecule.

FACS-Sorting of S. venezuelae Clones

The S. venezuelae exconjugant spores contaning clone 1B, as well as pJO436 vector, are FACS-sorted in 48-well, 96-well, and 384-well format into corresponding plates containing MYM agar+Apramycin 50 ug/ml. The single spore clones were allowed to germinate, grow and sporulate for 4-5 days.

Natural product extraction procedure: After the clones were fully grown and sporulated for 4-5 days, following volumes of solvent methanol were added to the each well containing the clones.

48 well format:0.8 ml

96 well format: 0.100 ml

384 well format: 0.06 ml

The plates were incubated at room temperature overnight.

The next day, the following volumes were recovered from the wells containing the clones.

48 well format: 0.3 ml

96 well format: 0.060 ml

384 well format: 0.030 ml

The extracts were assayed from a single well, and after combining extracts from 2, 4 and 10 wells.

The methanol extract was dried and resuspended in 40 ul of methanol:water and 20 ul of which was assayed against M. luteus as the indicator strain.

A single colony of S. venezuelae containing clone 1B produced enough bioactive molecule, in 48-well, 96-well as well as 384-well format, to be extracted by the microextraction procedure and to be detected by bioassay.

### EXAMPLE 11

### Expression of Actinorhodin Pathway in S. venezuelae 10712

When Sau3A pIJ2303 library constructed in pJO436 was introduced into S. venezuelae, one exconjugant which appeared blue-grey in color was spotted. This exconjugant showed blue pigment on R2-S agar suggesting the successful expression of a heterolgous pathway (actinorhodin) pathway in S. venezuelae. JO436 Segregational stability of S. venezuelae 10712 (pJO436::actinorhodin)

Since Streptomyces clones for small molecule production are grown in absence of antibiotic selection, it was important to determine how stable the S. venezuelae pJO436 recombinant clones are. The S. venezuelae 10712 (pJO436::actinorhodin) clone was used as an example.

The act clone was grown in R2-S liquid cultures with and without apramycin and total cell count was done by plating on R2-S agar with and without apramycin. The act clone gave 100% and 96% apramycin resistant colonies when grown with and without apramycin, respectively. This suggests that S. venezuelae pJO436 clones are quite stable segregationally.

Expression Stability of S. venezuelae 10712 (pJO436::actinorhodin)

We have shown successful expression of the actinorhodin gene cluster in S. venezuelae 10712. However, when this clone was grown in liquid cultures it failed to produce actinorhodin, as determined by the absence of its blue color. Nonetheless, when mycelia from such cultures were plated on solid media, actinorhodin producing colonies were clearly evident. The majority of the colonies produced a faint blue color while a few colonies produced abundant actinorhodin. These colonies which produce actinorhodin abundantly have been named as HBC (hyper blue clones) clones.

These observations suggest that perhaps in HBC clones, a host mutation has occurred which allows very efficient actinorhodin expression. Mutations which could lead to efficient actinorhodin expression could include a variety of targets such as, elimination of negative regulators like cutRS, overexpression of positive regulators, or efficient expression of pathways which provide precursors for actinorhodin. The hyper production of actinorhodin by the HBC clones thus strongly suggests that it is indeed possible for us to construct a strain which is more optimized for heterologous expression of small molecules, by random mutagenesis or by specific cutRS knockout mutagenesis.

Construction of a Jadomycin Blocked Mutant of S. venezuelae

Orf1 of the jadomycin biosynthetic gene cluster was chosen as a target. Primers were designed so as to amplify jad-L and jad-R fragments with proper restriction sites for future subcloning. S. venezuelae is reasonably sensitive to hygromycin and therefore, hygromycin resistance gene will be used to disrupt the orf-1 gene. The strategy used for disrupting the jadomycin orf-1 is described in the attached figure. The hyg-disrupted copy of the orf-1 gene will then be placed on pKC1218 and used for gene replacement in the S. venezuelae 10712, as well as VS153 chromosome.

Expression of the Yellow Clone in S. venezuelae

The single arm rescue technique to recover the yellow clone insert from S. lividans clone 525Sm575 was described. The recovered clone #3 was mated into S. venezuelae 10712 as well as VS 153. Yellow color was evident after several days on both 10712 as well as VS 153 plates but absent in the pJO436 vector alone controls. Three 10712 yellow clones were grown in liquid R2-S medium and all three produced yellow color profusely. This experiment has validated S. venezuelae as a host and pJO436 as the vector for heterologous expression for the second time, the first time being with the actinorhodin gene cluster. This yellow clone insert could now be used in validation of different strains in our strain improvement program.

3. Development of a Mating Protocol in a Microtiter Plate Format.

In order to have the individual E. coli donor clones archived, we are attempting to develop a mating protocol in a microtitre plate format. According to this protocol, we plan to sort the E. coli library into a 96-well microtitre plate. The matings with S. diversa would then be done in on a R2-S agar plate in an array format corresponding to the 96-well microtitre plate containing the E. coli clones. The bioassays can be either conducted on the mating R2-S plate or the clones can be first replica plated on to another suitable agar plate and then bioassayed. This approach will allow us to go back to the E. coli clones once we detect a bioactive clone among the S. diversa exconjugant library. The E. coli clone can then be mated back into S. diversa for re-transformation and confirmation of the bioactivity.

In a preliminary experiment, matings were done by spotting S. diversa spores together with E. coli donor cells on R2-S agar plate (rather than spreading). After about 8 hours the plate was overlayed as usual with apramycin and nalidixic acid. The exconjugants appeared only on those spots were E. coli donor was added, but not on those spots containing S. diversa spores alone. These initial data are very promising, although some more standardization needs to be done to develop this technique fully.

### EXAMPLE 12

### Production of Single Cells or Fragmented Mycelia

In order to produce single cells or fragmented mycelia, 25 ml MYM media was inoculated (see recipe below) in 250 ml baffled flask with 100 ul of Streptomyces 10712 spore suspension and incubated overnight at 30° C. 250 rpm. After a 24 hour incubation, 10 ml was transferred to 50 ml conical polypropylene centrifuge tube and centrifuged at 4,000 rpm for 10 minutes@25° C. Supernatant was decanted and the pellet was resuspended in 10 ml 0.05M TES buffer. The cells were sorted into MYM agar plates (sort 1 cell per drop, 5 cells per drop, 10 cells per drop) and we incubated the plates at 30° C.

MYM media (Stuttard, 1982, J. Gen. Microbiol. 128:115-121) contains: 4 g maltose, 10 g malt ext., 4 g yeast extract, 20 g agar, pH 7.3, water to 1 L.

### EXAMPLE 13

The following describes a method for the discovery of novel enzymes requiring large substrates (e.g., cellulases, amylases, xylanases) using the ultra high throughput capacity of the flow cytometer. As these substrates are too large to get into a bacterial cell, a strategy other than single intracellular detection must be employed in order to use the flow cytometer. For this purpose, we have adapted the gel microdrop (GMD) technology (One Cell Systems, Inc.) Specifically, the enzyme substrate is captured within the GMD and the enzyme allowed to hydrolyze the substrate within this microenvironment. However, this method is not limited to any particular gel microdrop technology. Any microdrop-forming material that can be derivatized with a capture molecule can be used. The basic experimental design is as follows: Encapsulate individual bacteria containing DNA libraries within the GMDs and allow the bacteria to grow to a colony size containing hundreds to thousands of cells each. The GMDs are made with agarose derivatized with biotin, which is commercially available (One Cell Systems). After appropriate colony growth, streptavidin is added to serve as a bridge between a biotinylated substrate and the biotin-labeled agarose. Finally, the biotinylated substrate will be added to the GMD and captured within the GMD through the biotin-streptavidin-biotin bridge. The bacterial cells will be lysed and the enzyme released from the cells. The enzyme will catalyze the hydrolysis of the substrate, thereby increasing the fluorescence of the substrate within the GMD. The fluorescent substrate will be retained within GMD through the biotin-streptavidin-biotin bridge and thus, will allow isolation of the GMD based on fluorescence using the flow cytometer. The entire microdrop will be sorted and the DNA from the bacterial colony recovered using PCR techniques. This technique can be applied to the discovery of any enzyme that hydrolyzes a substrate with the result of an increased fluorescence. Examples include but are not limited to glycosidases, proteases, lipases, ferullic acid esterases, secondary amidases, and the like.

One system uses a biotin capture system to retain secreted antibodies within the GMD. The system is designed to isolate hybridomas that secrete high levels of a desired antibody. This basic design is to form a biotin-streptavidin-biotin sandwich using the biotinylated agarose, streptavidin, and a biotinylated capture antibody that recognizes the secreted antibody. The “captured” antibody is detected by a fluoresceinated reporter antibody. The flow cytometer is then used to isolate the microdrop based on increased fluorescence intensity. The potentially unique aspect to the method described here is the use of large fluorogenic substrates for the determination of enzyme activity within the GMD. Additionally, this example uses bacterial cells containing DNA libraries instead of eukaryotic cells and is not confined to secreted proteins as the bacterial cells will be lysed to allow access to the enzymes.

The fluorogenic substrates can be easily tailored to the particular enzyme of interest. Described below is a specific example of the chemical synthesis of an esterase substrate. Additionally, two examples are given which describe the different possible chemical combinations that can be used to make a wide variety of substrates.

Example of Reaction Sequence Leading to GMD-Attachable Substrate

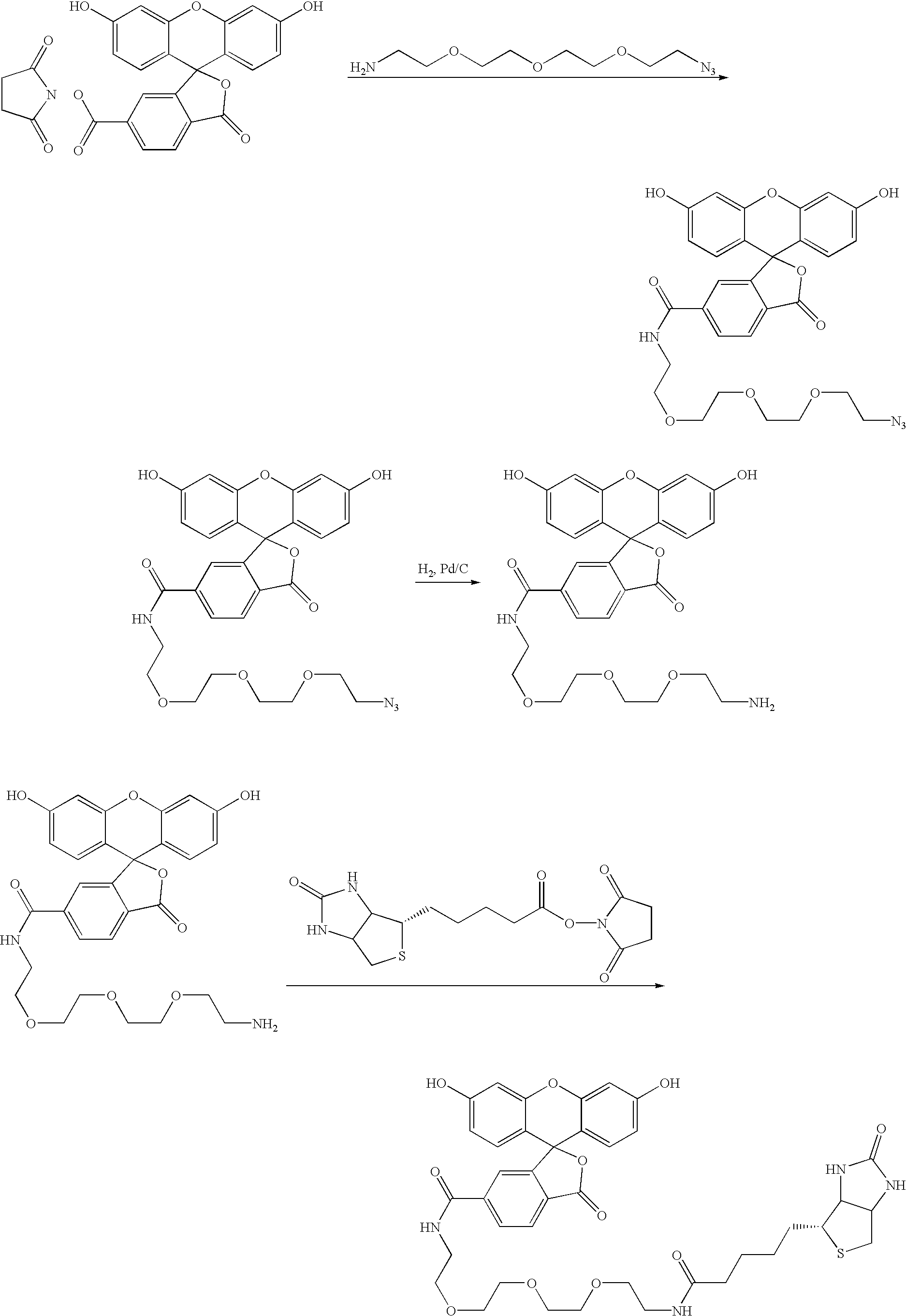

In the first step, 1-amino-11-azido-3,6,9-trioxaundecane [Reference 3], an asymmetric spacer, is attached to N-hydroxysuccinamide ester of 5-carboxyfluorescein (Molecular Probes). After reduction of the azide functional group on the end of the attached spacer (step 2), activated biotin (Molecular Probes) is attached to the amine terminus (step 3), and the sequence is completed by esterification of phenolic groups of the fluorescein moety (step 4). The resulting compound can be used as a substrate in screens for esterase activity.

Design of GMD-Attachable Fluorogenic Substrates

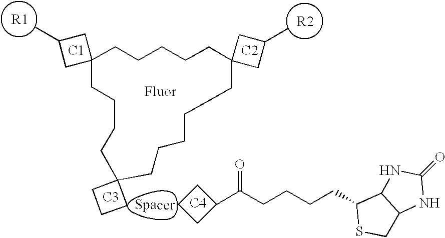

Fluor—core fluorophore structure, capable of forming fluorogenic derivatives, e.g. coumarins, resorufins, xanthenes, and others.

Spacer—a chemically inert moiety providing connection between biotin moiety and the fluorophore. Examples include alkanes and oligoethyleneglycols. The choice of the type and length of the spacer will affect synthetic routes to the desired products, physical properties of the products (such as solubility in various solvents), and the ability of biotin to bind to deep pockets in avidin.

C1, C2, C3, C4—connector units, providing covalent links between the core fluorophore structure and other moieties. C1 and C2 affect the specificity of the substrates towards different enzymes. C3 and C4 determine stability of the desired product and synthetic routes to it. Examples include ether, amine, amide, ester, urea, thiourea, and other moieties.

R1 and R2—functional groups, attachment of which provides for quenching of fluorescence of the fluorophore. These groups determine the specificity of substrates towards different enzymes. Examples include straight and branched alkanes, mono- and oligosaccharides, unsaturated hydrocarbons and aromatic groups.

a. Design of GMD-Attachable Fluorescence Resonance Energy Transfer Substrates

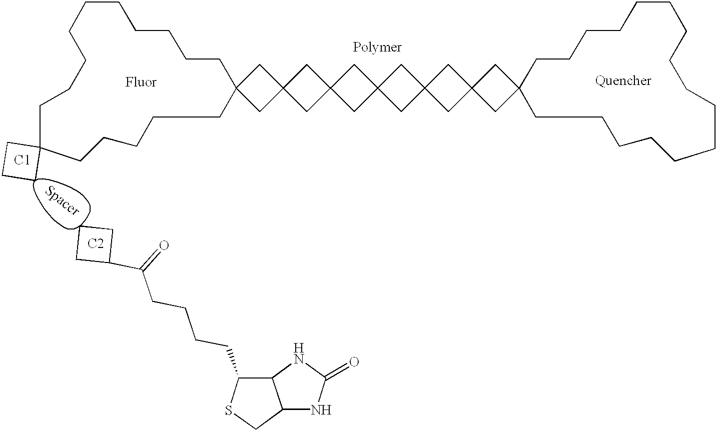

Fluor—A fluorophore. Examples include acridines, coumarins, fluorescein, rhodamine, BODIPY, resorufin, porphyrins, etc.

Quencher—A moiety, which is capable of quenching fluorescence of the fluorophore when located at a close enough distance. Quencher can be the same moiety as the fluorophore or a different one.

Polymer is a moiety, consisting of several blocks, a bond between which can be cleaved by an enzyme. Examples include amines, ethers, esters, amides, peptides, and oligosaccharides,

C1 and C2 are equivalent to C3 and C4 in the previous design.

Spacer is equivalent to Spacer in the previous design.

### REFERENCES

[1] Gray, F, Kenney, J. S., Dunne, J. F. Secretion capture and report web: use of affinity derivatized agarose microdroplets for the selection of hybridoma cells. J Immunol. Meth. 1995,182,155-163.

[2] Powell, K. T. and Weaver, J. C. Gel microdroplets and flow cytometry: Rapid determination of antibody secretion by individual cells within a cell population. Bio/technology 1990, 8, 333-337.

[3] Schwabacher, A. W.; Lane, J. W.; Schiesher, M. W.; Leigh, K. M.; Johnson, C. W. J. Org. Chem. 1998, 63, 1727-1729.

### EXAMPLE 14

The goal of this experiment is to develop an ultra high throughput screen designed for discovery of novel anticancer agents. In contrast to the traditional combinatorial chemistry or natural product extract approach. The method of Example 14 uses a recombinant approach to the discovery of bioactive molecules. The examples use complex DNA libraries from a mixed population of uncultured microorganisms that provide a vast source of natural products through recombinant expression from whole gene pathways. The two objectives of this Example include:

1) Engineering of mammalian cell lines as reporter cells for cancer targets to be used in ultra-high throughput assay system.

2) Detection of novel anticancer agents using an ultra high throughput FACS-based screening format.

The present invention provides a new paradigm for screening technologies that brings the small molecule libraries and target together in a three dimensional ultra high throughput screen using the flow cytometer. In this format, it is possible to achieve screening rates of up to 108 per day. The feasibility of this system is tested using assays focused on the discovery of novel anti-cancer agents in the areas of signal transduction and apoptosis. Development of a validated assay should have a profound impact on the rate of discovery of novel lead compounds.

Experimental Design and Methods

1. Development of Cell Lines

The goal of this example is to develop an ultra high throughput screening format that can be used to discover novel chemotherapeutic agents active against a range of molecular targets known to be important in cancers. The feasibility of this approach will be tested using mammalian cell lines that respond to activation of the epidermal growth factor receptor (EGFR) with induction of expression of a reporter protein. The EGFR-responsive cells will be brought together with our microbial expression host within a microdrop (see Example 13 and co-pending U.S. Pat. No. 6,280,926, and U.S. application Ser. No. 09/894,956, both herein incorporated by reference). These expression hosts will be Streptomyces or E coli and will contain libraries derived from a mixed population of organisms, i.e. high molecular weight environmental DNA (10-100 kb fragments) cloned into the appropriate vectors and transferred to the host. These large DNA fragments will contain biosynthetic operons which consist of the genes necessary to produce a bioactive small molecule. A bioactive molecule from the microbial host will elicit a biological response in the mammalian cell which will induce expression of a fluorescent reporter. The entire microdrop will be individually sorted on the flow cytometer based on fluorescence and the DNA from the host recovered. The mixed population libraries may contain from 10⁴-10¹⁰ clones, including 10⁵, 10⁶, 10⁷, 10⁸, 10⁹, or any multiple thereof.

An assay based on the EGF receptor was chosen because of its possible role in the pathogenesis of several human cancers. The EGF-mediated signal transduction pathway is very well characterized and several inhibitors of the EGF receptor have been found from natural sources (21,22). The EGFR is one of the early oncogenes discovered (erbB) from the avian erythroblastosis retrovirus and due to a deletion of nearly all of the extracellular domain, is constitutively active (23). Similar types of mutations have been found in 20-30% of cases of glioblastoma multiforme, a major human brain tumor (24).

Overexpression of EGFR correlates with a poor prognosis in bladder cancer (25), breast cancer (26,27), and glioblastoma multiforme (28). Most of these cancers occur in an EGF-secreting background and suggests an autocrine growth mechanism in these cancers. Additionally, EGFR is overexpressed in 40-80% of non-small cell lung cancers and EGF is overexpressed in half of primary lung cancers, with patient prognosis significantly reduced in cases with concurrent expression of EGFR and EGF (29,30). For these reasons, inhibitors of the EGF receptor are potentially useful as chemotherapeutic agents for the treatment of these cancers.

The goal of this experiment is to create mammalian cell lines that serve as reporter cells for anticancer agents. HeLa cells endogenously express the EGFR as confirmed by FACS analysis using the anti-EGFR antibody, Ab-1 (Calbiochem). In contrast, CHO cells have little or no expression of the EGFR. The gene encoding EGFR was obtained from Dr. Gordon Gill (University of California, San Diego) and cloned it into the pcDNA3/hygro vector. The resulting vector was transfected into CHO cells and stable transformants selected with hygromycin. Enrichment of high EGFR-expressing CHO cells was performed through two rounds of FACS sorting using the anti-EGFR antibody. For detection of the activated pathway, a parallel approach is being taken utilizing both the PathDetect system from Stratagene (San Diego, Calif.) and the Mercury Profiling system from Clontech (San Diego, Calif.). The Path Detect system has been validated by researchers as a means of detecting mitogenic stimuli (31,32).

The EGFR is a tyrosine kinase receptor that functions through the MAP-kinase pathway to activate the transcription factor Elk-1 (33). The PathDetect product includes a fusion trans-activator plasmid (pFA-Elk1) that encodes for expression of a fusion protein containing the activation domain of the Elk-1 transcription activator and the DNA binding domain of the yeast GAL4. A second plasmid contains a synthetic promoter with five tandem repeats of the yeast GAL4 binding sites that control expression of the Photinus pyralis luciferase gene. The luciferase gene was removed and replaced with the gene encoding for the destabilized version of the enhanced green fluorescent protein (EGFP) (plasmid designated pFR-d2EGFP). The two plasmids were transfected together into the EGFR/CHO and HeLa cells at a ratio of 10:1 (pFR-EGFP: pFA-Elk1) and stable transformants selected using the neomycin resistance gene located on the pFA-Elk1 plasmid. Thus, ligand binding to the EGFR will initiate a signal transduction cascade that results in activation of the Elk1 portion of the fusion protein, allowing the DNA binding domain of the yeast GAL4 to bind to its promoter and turn on expression of EGFP.

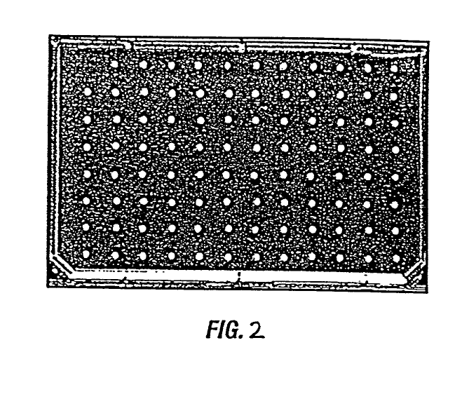

Stimulation in the presence of serum is not surprising as this signal transduction pathway is common to most growth factors and it is likely that many growth factors including EGF are present in the serum. After 24 hours of significant serum starvation, this response is greatly reduced (FIG. 2A). The next step will be to selectively stimulate these cells with recombinant EGF (Calbiochem) and isolate the highly responsive single clones using the flow cytometer. These clones will be selected by sorting simultaneously for high levels of GFP and the EGFR. The EGFR will be detected using an anti-EGFR antibody with a secondary antibody labeled with phycoerythrin. This system has the advantage that use of the yeast GAL4 promoter in these cells should keep background or spurious induction of EGFP to a minimum. The second group of cell lines uses the Mercury Profiling system to assay the same EGFR pathway. This system responds to activation of the pathway with an increase in the expression of human placental secreted alkaline phosphatase (SEAP). A fluorescent signal will be obtained by the addition of the phosphatase substrate ELF-97-phosphate (Molecular Probes), which yields a bright fluorescent precipitate upon cleavage. The advantage of this approach over the PathDetect system is the ability to amplify the signal through enzyme catalysis for low-level activation of the pathway. This parallel approach will increase the probability of success in finding bioactive compounds. In the Mercury Profiling system, a vector containing the cis-acting enhancer element SRE and the TATA box from the thymidine kinase promoter is used to drive expression of alkaline phosphatase (pTA-SEAP). This system relies on the endogenous transactivators present in the cell, such as Elk-1, to bind the SRE element on the vector and drive expression of SEAP upon stimulation of EGFR. The pTA-SEAP vector was transfected into the EGFR/CHO and HeLa cells and stable transformants selected using neomycin. Again, stimulation of the pathway occurred in the presence of serum factors in the media. Upon serum starvation, this response was greatly reduced (FIG. 2B). Single high expressing clones will be isolated following stimulation with EGF and sorting using a flow cytometer.

Development of Ultra High Throughput FACS Assay

We have generated complex mixed population libraries (>10⁶ primary clones/library) that provide access to the untapped biodiversity that exist in the >99% uncultivable microorganisms. These novel libraries require the development of ultra high throughput screening methods to obtain complete coverage of the library. We propose developing an assay using the flow cytometer that allows detection of up to 10⁸ clones/day.

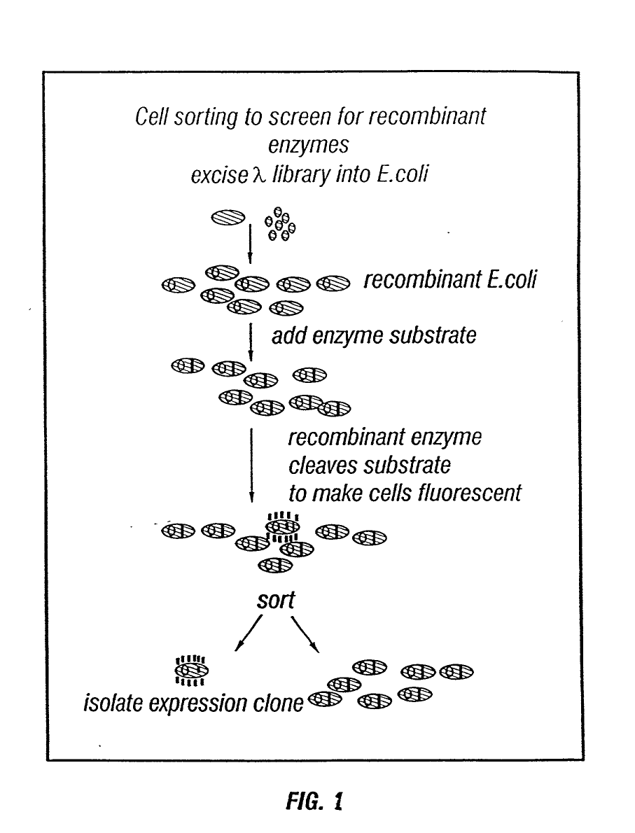

In this assay format (FIG. 1), an expression host (Streptomyces, E. coli) and a mammalian reporter cell will be co-encapsulated together within a microdrop. The microdrop holds the cells in close proximity to each other and provide a microenvironment that facilitates the exchange of biomolecules between the two cell types. The reporter cell will have a fluorescent readout and the entire microdrop will be run through the flow cytometer for clonal isolation. The DNA from the genes or pathway of interest will subsequently be recovered using in vitro molecular techniques. This assay format will be validated for the discovery of both EGFR inhibitors as well as for small molecules that induce apoptosis. With validation of this format, we will progress to the ultra high throughput screening phase designed to discover novel chemotherapeutic agents active against these important molecular mechanisms underlying tumorigenesis.

The feasibility of this approach will be analyzed initially using the engineered cell lines described above that respond to activation by EGF with increased expression of a reporter protein (i.e. EGFP or alkaline phosphatase). Additionally, this initial study will use an E. coli host that overexpresses human EGF as a secreted protein directed to the bacterial periplasm (34). This approach will allow us to validate the assay format prior to screening for inhibitors of the EGFR pathway using our E. coli and Streptomyces expression libraries. For this experiment, the engineered cell lines will be co-encapsulated together with the E. coli host at a ratio of one to one. The EGF-expressing bacteria will be allowed to grow and form a colony within the microdrop. Due to the vastly higher growth rate of bacteria, a colony of bacteria will form prior to any or minimal cell division of the eukaryotic cell. This colony will then provide a significantly increased concentration of the bioactive molecule. The bacterial colony will be selectively lysed using the antibiotic polymyxin at a concentration that allows cell survival (35). This antibiotic acts to perforate bacterial cell walls and should result in the release of EGF from these cells without affecting the eukaryotic cell. In the final discovery assays, this lysis treatment should not be necessary as the small molecule products will likely be able to freely diffuse out of the cell. The EGF will activate the signal transduction pathway in the eukaryotic cell and turn on expression of the reporter protein.

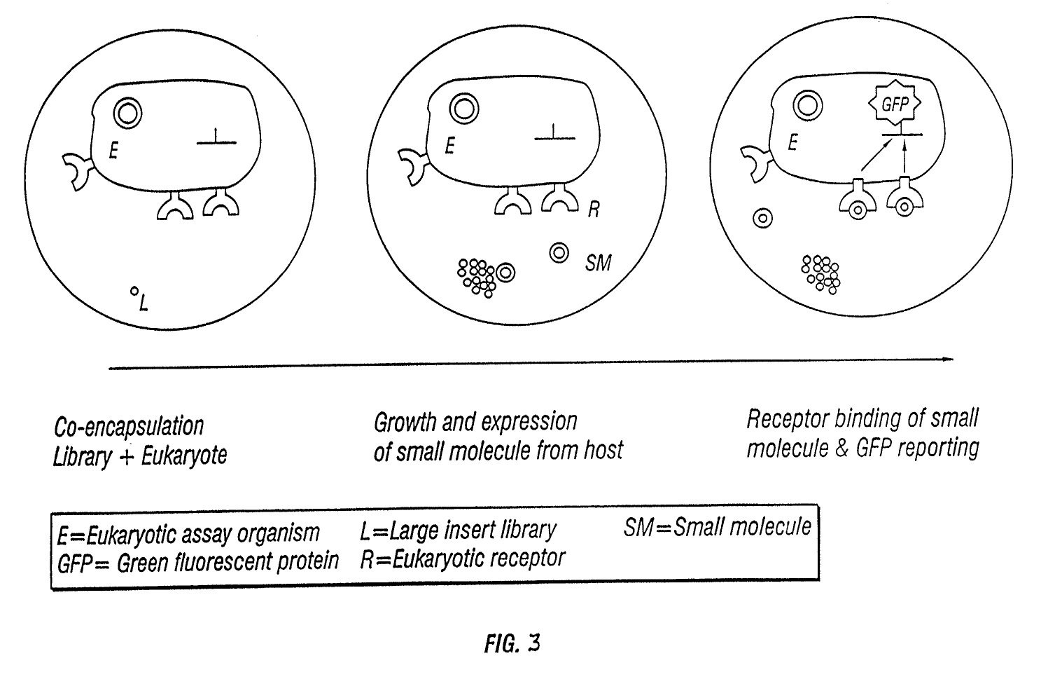

The microdrops will be run through the flow cytometer and those microdrops exhibiting an increased fluorescence will be sorted. The DNA from the sorted microdrops will be recovered using PCR amplification of the insert encoding for EGF. For the reporter cells expressing secreted alkaline phosphatase, a couple of additional steps are required to achieve a fluorescent readout. As the enzyme is secreted from the cell, it is possible to prevent the diffusion of the protein from the microdrop by selectively capturing it within the matrix of the microdrop. This can be accomplished by using microdrops made with agarose derivatized with biotin. By forming a sandwich with streptavidin and a biotinylated anti-alkaline phosphatase antibody, it is possible to capture alkaline phosphatase where it can catalyze the conversion of the ELF-97 phosphate substrate within the microdrop (FIG. 3A). This technique was successfully developed by One Cell Systems for the isolation of high expressing hybridomas (36,37). In our hands, with the encapsulation of the SEAP expressing cells, we have shown that upon addition of the Elf-97 phosphatase substrate, a fluorescent precipitate forms within the microdrop (FIGS. 3B&C).

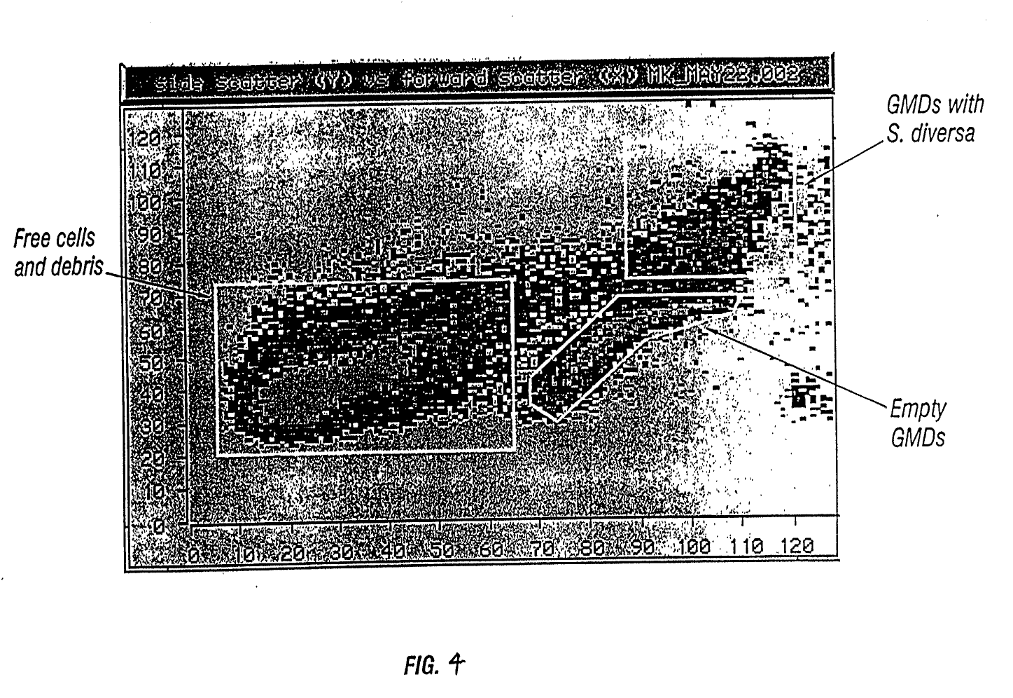

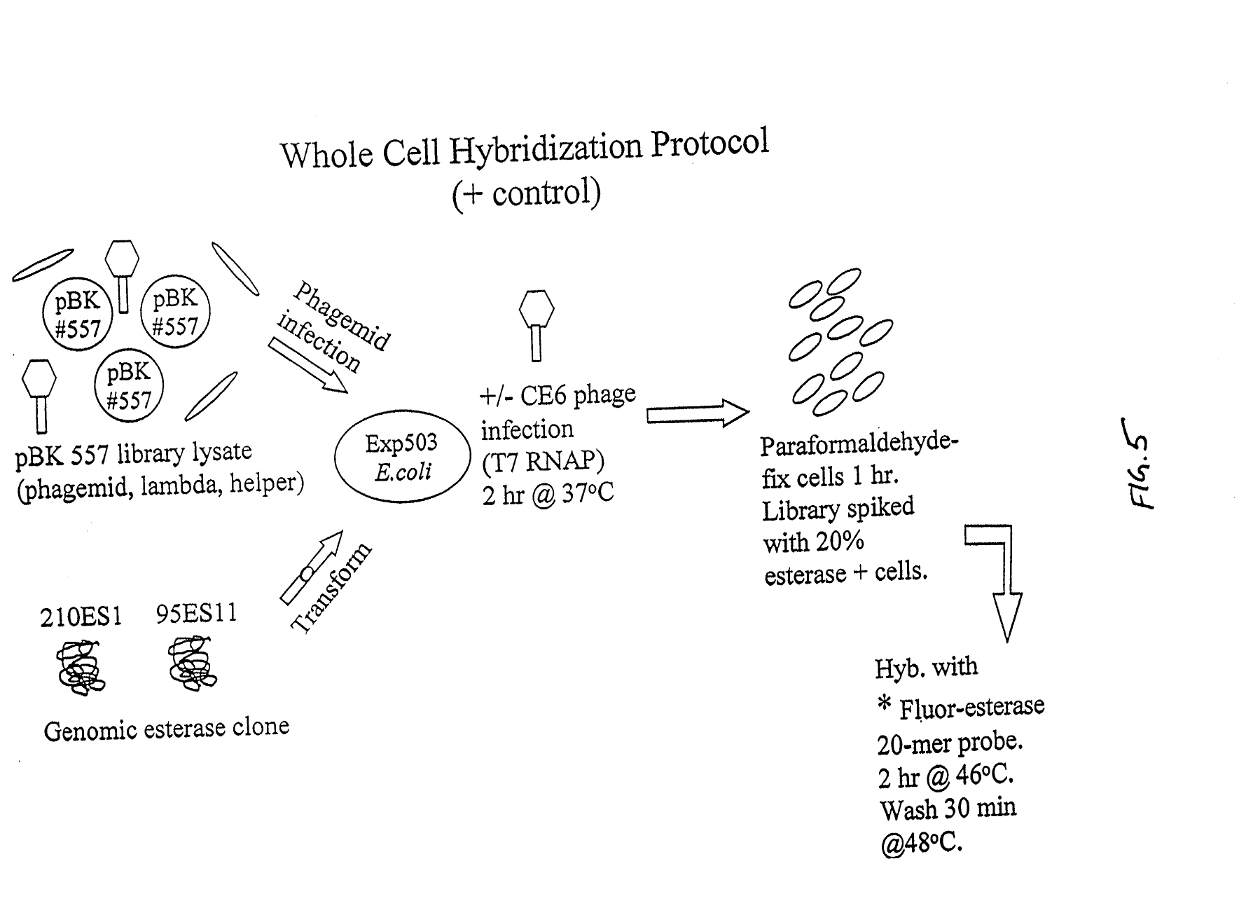

Initial experiments demonstrate the feasibility of co-encapsulating E. coli and mammalian cells (e.g., CHO) within microdrops. Microdrops were formed using 3% agarose dropped in oil and blended at 2600 rpm. The E. coli and CHO cells were encapsulated at a ratio of 1:1 (FIG. 4A). After 6 hours, the single bacterial cell grew into a colony containing thousands of cells (FIG. 4B). The cells within the microdrops were stained with propidium iodide to determine viability and approximately 70-85% of the CHO cells remained viable after 24 hours. Subsequent steps include determining the response of encapsulated clonal EGF-responsive mammalian cells to varying concentrations of EGF in the presence and absence of EGFR inhibitors such as Tyrphostin A46 or Tyrphostin A48 (Calbiochem). In addition, E. coli clones producing high levels of secreted EGF will be isolated using the Quantikine human EGF immunoassay (R&D Systems). Finally, these two cell types will be brought together within the microdrop and a change in fluorescence of the eukaryotic cell will be analyzed on the flow cytometer in the presence and absence of the EGFR inhibitors. A positive result in this experiment would be an increase in fluorescence that can be blocked by the EGFR inhibitors.

The next step will be to mix the EGF-expressing E. coli with non-expressing cells at varying ratios from 1:1,000 to 1:1,000,000 to mimic the conditions of an mixed population library discovery screen. The bacterial mixtures and the mammalian cells will be co-encapsulated as described above. The highly fluorescent microdrops will be individually sorted by the flow cytometer. To confirm a positive hit, the DNA will be recovered by PCR amplification using primers directed against the EGF gene. To improve the signal to noise ratio, it is likely that it will be necessary to undergo several rounds of enrichment before isolation of positive EGF-expressing clones, especially for the higher mixture ratios.

In this case, the microdrops will first be sorted in bulk, the microdrop material removed with GELase (Epicentre Technologies) and the bacteria allowed to grow. The encapsulation protocol will be repeated with fresh eukaryotic cells until a highly enriched population is observed. At this point, single microdrops will be isolated and recovery of the EGF-expressing clone confirmed by PCR. With validation of this assay, the goal will be to screen for inhibitors of the EGFR using our mixed population libraries expressed in optimized E. coli and Streptomyces hosts. This assay will be done in the presence of EGF and the assay endpoint will be a decrease in fluorescence. This format is not limited to only EGFR inhibitors as any protein within this pathway could be inhibited and would appear positive in this screen. Likewise, this screen can also be adapted to the multitude of anti-cancer targets that are known to regulate gene expression. In fact, using this present system, with the addition of the appropriate receptors, it would be possible to screen for inhibitors of other growth factors such as PDGF and VEGF.

If an increase in fluorescence is not observed with co-encapsulation of the EGF-expressing cells and the mammalian reporter cell, there could be several reasons. First, it is possible that the EGF diffuses out of the cell too quickly to elicit a response. In this case, it will be necessary to modify the microdrops to limit diffusion and concentrate the bioactive molecule at the site of the reporter cell. It is also possible that in the specific case of the EGF assay, the cells will not continue to produce EGF after polymyxin treatment and thus, the incubation time of the reporter cells with EGF will be minimal. This is unlikely as the polymyxin treatment used will be at concentrations well below that which produces decreased cell viability. However, if EGF is not continually expressed in this system, other permeabilization methods will be explored that do not significantly affect cell metabolism, such as the bacteriocin release protein (BRP) system (Display Systems Biotech). The BRP opens the inner and outer membranes of E. coli in a controlled manner enabling protein release into the culture medium. This system can be used for large-scale protein production in a continuous culture and thus should be compatible with cell survival.

Apoptosis, or programmed cell death, is the process by which the cell undergoes genetically determined death in a predictable and reproducible sequence. This process is associated with distinct morphological and biochemical changes that distinguish apoptosis from necrosis. The malfunctioning of this essential process can often lead to cancer by allowing cells to proliferate when they should either self-destruct or stop dividing. Thus, the mechanisms underlying apoptosis are currently under intense scrutiny from the research community and the search for agents that induce apoptosis is a very active area of discovery.

The present invention provides to develop an assay for the discovery of apoptotic molecules using our ultra high throughput encapsulation technology. The source of these small molecules will come from our extremely complex mixed population libraries expressed in Streptomyces and E. coli host strains. These host strains will be co-encapsulated together with a eukaryotic reporter cell, the small molecule will be produced in the bacterial strain, and will act on the mammalian reporter cell which will respond by induction of apoptosis. Apoptosis will be detected using a fluorescent marker, the entire microdrop sorted using the flow cytometer, and the DNA of interest recovered. The feasibility of this assay will be determined using our optimized Streptomyces host strain, S. diversa, co-encapsulated with the apoptotic reporter cell derived from human T cell leukemia (e.g., Jurkat cells). The pathway controlling production of the anti-tumor antibiotic, bleomycin, will be cloned into S. diversa as the source of an apoptosis-inducing agent. The readout for induction of apoptosis in Jurkat cells will be obtained using the fluorescent marker, Alexis 488-annexin V.

The bleomycin group of compounds are anti-tumor antibiotics that are currently being used clinically in the treatment of several types of tumors, notably squamous cell carcinomas and malignant lymphomas. However, widespread use of bleomycin congeners has been limited due to early drug resistance and the pulmonary toxicity that develops concurrent with administration of this drug. Thus, there is continuing effort to find novel small molecules with better clinical efficacy and lower toxicity. Bleomycin congeners are peptide/polyketide metabolites that function by binding to sequence selective regions of DNA and creating single and double stranded DNA breaks. Several in vitro and in vivo assays have shown that bleomycin induces apoptosis in eukaryotic cells (43-45). The biosynthetic gene cluster encoding for the production of bleomycin has recently been cloned from Streptomyces verticillus and is encoded on a contiguous 85 kb fragment (46). We propose to clone this pathway into a BAC vector to use as a source of apoptotic agents in eukaryotic cells. A library will be made from the S. verticillus ATCC15003 strain and cloned into the BAC vector, pBlumate2. As the sequence for this pathway is known, probes will be designed against sequences from the 5′ and 3′ ends of the pathway. The library will be introduced into E. coli and screened using colony hybridization with the probe generated against one end of the pathway. Positive clones will subsequently be screened with the second probe to identify which clone contains the entire pathway. Clones containing the complete pathway will be transferred into our optimized expression host S. diversa by mating. Expression of bleomycin will be detected using whole cell bioassays with Bacillus subtillis.

Jurkat cells are the classic human cell line used for studies of apoptosis. The fluorescent Alexis 488 conjugate of annexin V (Molecular Probes) will be used as the marker of apoptosis in these cells. Annexin V binds to phosphotidylserine molecules normally located on the internal portion of the membrane in healthy cells. During early apoptosis, this molecule flips to the outer leaf of the membrane and can be detected on the cell surface using fluorescent markers such as the annexin V-conjugates. The bleomycin-induced apoptotic response in Jurkat cells will initially be characterized by varying both the concentrations of the exogenously administered drug and the incubation time with the drug. Alexis 488-annexin V will then be add to the cells and the level of fluorescence analyzed on the flow cytometer. Necrotic cell death will be determined using propidium iodide and the apoptotic population will be normalized to this value.

Co-encapsulation of S. diversa with CHO cells within microdrops produced very similar results to the E. coli co-encapsulation. S. diversa grew well in the eukaryotic media and the CHO cell survival rate was high after 24 hours. In this experiment, the S. diversa clone expressing bleomycin will be co-encapsulated with the Jurkat cell line. S. diversa will be allowed to grow into a colony within the microdrop and begin production of bleomycin. The microdrops will be periodically analyzed over time for induction of apoptosis using the Alexis 488-annexin V conjugate on the microscope and flow cytometer. After noting the time for induction of apoptosis, a mixing experiment similar to that described for the EGF experiment will be performed. Bleomycin-expressing and non-expressing cells will be mixed together at ratios of 1:1000 to 1:1,000,000. Co-encapsulation of the mixtures with Jurkat cells will be performed and the appropriate incubation time maintained. These microdrops will then be stained with Alexis 488-annexin V and sorted on the flow cytometer. Confirmation of a positive bleomycin-expressing sorted clone will be performed by PCR amplification of a portion of the pathway. Again, it is likely that enrichment of these mixtures will be necessary using a few rounds of bulking sorting on the flow cytometer.

If no apoptosis is observed in the initial assay, confirmation of bleomycin production will be performed by sorting of the encapsulated S. diversa clone into 1536 well plates. After a predetermined incubation period, the supernatent will be removed and spotted on filter disks for whole cell bioassays using the susceptible strain B. subtilis. Use of the 1536 well plates will hopefully avoid significant dilution of the antibiotic in the media. As cloning of the bleomycin pathway is quite recent, it has not yet been heterologously expressed from the complete pathway. However, Du et al demonstrated the heterologous bioconversion of the inactive aglycones into active bleomycin congeners by cloning a portion of the pathway into a S. lividans host (46). If bleomycin expression is not detectable in our assay, we will employ a similar strategy using our host strain S. diversa. If little bleomcyin production is detected under these conditions, it will be necessary to optimize the culture conditions for S. diversa to induce pathway expression within the microdrop. On the other hand, if bleomycin is produced but apoptosis is not observed, it is possible that the molecule is diffusing away from the microdrop too quickly and it will be necessary to optimize the microdrop technology to concentrate the metabolite at the site of the reporter cell.

Optimization of S. diversa Secondary Metabolite Expression in Microdrop

Induction of pathway expression is an issue that is not limited to the bleomycin example. Bioactive small molecules within microorganisms are often produced to increase the host's ability to survive and proliferate. These compounds are generally thought to be nonessential for growth of the organism and are synthesized with the aid of genes involved in intermediary metabolism, hence the name “secondary metabolites.” Thus, the pathways controlling expression of these secondary metabolites are often regulated under non-optimal conditions such as stress or nutrient limitation. As our system relies on use of the endogenous promoters and regulators, it might be necessary to optimize conditions for maximal pathway expression.

There are several methods that can used to optimize for increased pathway expression within the microdrops. For easy detection of maximal expression, we will construct a transposon containing a promoter-less GFP. The enhanced GFP optimized for eukaryotes will be used as it has a codon bias for high GC organisms. Transposition into a known pathway (e.g., actinorhodin) will be done in vitro and the vector containing the pathway purified. The transposants will be introduced into an E. coli host, screened for clones that express GFP, and positive clones isolated on the flow cytometer. With the transfer of the promoter-less gene for GFP into the pathway, increased fluorescence within the cells would suggest transcription of the pathway using the endogenous promoters located within the pathway. This clone will be used as a tool for quick detection of upregulation in pathway expression due to changes in the experimental conditions.

The S. diversa clone containing GFP and the actinorhodin pathway will be encapsulated in the microdrops and several different growth conditions will be tested, e.g., conditioned media, nutrient limiting media, known inducing factors, varying incubation times, etc. The microdrops will be analyzed under the microscope and on the flow cytometer to determine which conditions produce optimal expression of the pathway. These conditions will be verified for viability in eukaryotic cells as well. These optimized growth conditions will be confirmed using the bleomycin pathway to assess production of the secondary metabolite. Additionally, whole cell optimization of S. diversa is ongoing with production of strains that are missing different pleiotropic regulators that often negatively impact secondary metabolite production. As these strains are developed, they will be analyzed in the microdrops for enhanced pathway expression.

The proximity of the two cell types within the microdrop should result in a high concentration of the bioactive molecule at the site of the reporting cell. However, if rapid diffusion of the molecule from the microdrop prevents detection of the desired signal, it will be necessary to optimize the microdrop protocol or develop a new encapsulation technology. Concentration of the molecule at the site of the reporter cell could be achieved by a reduction in the microdrop pore size. Pore size reduction can be accomplished by one or a combination of the following approaches: (i) “plugging” the holes with particles of an appropriate size, which are held in the pores by non-covalent or covalent interactions; (ii) cross-linking of the microdrop-forming polymer with low molecular weight agents; (iii) creation of an external shell around the microdrop with pores of smaller size than those in the current microdrop.

(i) Plugging the pores can be accomplished using polydisperse latexes with particles sized to fit within the pores of the microdrop. Latex particles may be modified on their surface such that they are attracted to the microdrop-forming polymer. For example, agarose-based microdrops carry a negative electrostatic charge on the surface. Thus, amidine-modified polystyrene latex particles (Interfacial Dynamics Corporation) will be attracted to the microdrop surface and the latex particles will effectively plug the microdrop pores provided that the charge density on the latex particles and the microdrop surface is high enough to sustain strong electrostatic bonds.

(ii) Cross-linking of agarose beads can be achieved by treating them with various reagents according to known procedures (47). For our purposes, the cross-linking needs to occur only on the surface of microdrop. Thus, it may be advantageous to use polymers carrying reactive groups for cross-linking of agarose, such that permeation of the cross-linking agent inside the microdrop is prevented.

(iii) Formation of classical (48) or polymerizable liposomes (49,50) around microdrops would provide a shell that could be an effective barrier even to small molecules. A wide variety of precursors for such liposomes as well as methods for their preparation have been reported (48-50) and most of them are applicable for our purposes. One of the possible limitations in choice of precursors stems from the intended use of microdrops for eventual screening by the flow cytometer. Thus, the liposomes should not absorb in the visible part of the spectrum.

It might also be necessary to use alternative methods and materials for preparation of the microdrops. Encapsulation of cells in polyacrylamide, alginate, fibrin, and other gel-forming polymers has been described (51). Another plausible candidate for encapsulation material is silica gel, which can be formed under physiological conditions with the assistance of enzymes (silicateins) (52) or enzyme mimetics (53). Additionally, various polymers may be used as the material for microdrop construction. Microdrops may be formed either upon polymerization of monomers (i.e. water-soluble acrylates or metacrylates) or upon gelation and/or cross-linking of preformed polymers (polyacrylates, polymetacrylates, polyvinyl alcohol). Since the formation of microdrops occurs simultaneously with encapsulation of living cells, such formation has to proceed under conditions compatible with cell survival. Thus, the precursors for microdrops (monomers or non-gelated polymers) should be soluble in aqueous media at physiological conditions and capable of the transformation into the microdrop material without any significant participation and/or emission of toxic compounds.

### EXAMPLE 15

### Identification of a Novel Bioactivity or Biomolecule of Interest by Mass Spectroscopic Screening

An integrated method for the high throughput identification of novel compounds derived from large insert libraries by Liquid Chromotography—Mass Spectrometry was performed as described below.

A library from a mixed population of organisms was prepared. An extract of the library was collected. Extracts from the libraries were either pooled or kept separate. Control extracts, without a bioactivity or biomolecule of interest were also prepared.

Rapid chromatography was used with each extract, or combination of extracts to aid the ionization of the compound in the spectra. Mass spectra were generated for the natural product expression host (e.g. S. venezuelae) and vector alone (e.g. pJO436) system. Mass spectra were also generated for the host cells containing the library extracts, alone or pooled. The spectra generated from multiple runs of either the background samples or the library samples were combined within each set to create a composite spectra. Composite spectra may be generated by using a percentage occurrence of an average intensity of each binned mass per time period or by using multiple aligned single mass spectra over a time period. By using a redundant sampling method where each sample was measured several times in the presence of other extracts, the novel signals that consistently occurred within a sample extract but not within the background spectra were determined.

The host-vector background spectrum was compared to the mass spectra obtained from large insert library clone extracts. Extra peaks observed in the large insert library clone extracts were considered as novel compounds and the cultures responsible for the extracts were selected for scale culture so the compound can be isolated and identified.

Novel Metabolite Identification by Mass Spectroscopic Screening.

In integrated method for the high throughput identification of novel compounds derived from large insert libraries by LC-MS is described below. Liquid chromatography-mass spectrometry is used to determine the background mass spectra of the natural product expression host (e.g. S. diversa DS10 or DS4) and vector alone (e.g. pmf17) system. This host-vector background spectrum is compared to the mass spectra obtained from large insert library clone extracts. Extra peaks observed in the large insert library clone extracts are considered as novel compounds and the cultures responsible for the extracts are selected for scale culture so the compound can be isolated and identified.

In order to create the background and sample spectra, rapid chromatography is used to aid the ionization of the compounds in the extract. The spectra generated from multiple runs of either the background samples or the library samples are combined within each set to create a composite spectra. Composite spectra may be generated by using a percentage occurrence of an average intensity of each binned mass per time period or by using multiple aligned single mass spectra over a time period. Using a redundant sampling method where by each sample is measured several times in the presence of other extracts the novel signals that consistently occur within a sample extract but not present in the background spectra can be determined. The purpose of this invention is to identify novel compounds produced by recombinant genes encoding biosynthetic pathways without relying on the compounds having bioactivity. This detection method is expected to be more universal than bioactivity for identifying novel compounds.

Currently there is a similar method of examining culture mixtures by LC-MS with long chromatographic times (30-60 min) to bring compounds to a fairly high level of purity. This method relies on molecular weight searches for dereplication of known compounds. This slow method would also work to identify novel compounds in S. diversa libraries however the throughput would be inadequate for the number of samples we need to screen. There are a pair of publications describing rapid direct infusion analysis of samples to identify fermentation conditions which improve the biosynthetic productivity of strains. This method does not identify specific compound, it just correlates greater, more complex production with different culture conditions.

Shown below are the following:

1. Chromatographic gradient and mass spec conditions

HPLC and MS setting for Mass Spec Screening.TXT

2. Pooling of samples sheet

Sampling Strategy.htm

3. Sample flow using average method

Mass Spec Screening Flow chart.doc

4. Matlab code for original average background

Mass Spec Screening Summary6 Matlab code.txt

5. Matlab code under development for new single aligned peaks background determination for more accurate data analysis.

Mass Spec Screening 2nd Data Analysis Program.txt

The method is best practiced with a set of control extracts and sample extracts. Mixing of the compounds in pools prior to analysis and deconvolution of the mixed extract pools will provide high throughput while maintaining the ability to measure each extract several times.

A secondary screen may be required to eliminate false positives.

This method is more specific for identifying potential novel compounds by molecular ion than current methods. This method uses a different data analysis strategy than the dereplication methods for the identification of specific peaks for new compounds in extracts. Using the molecular ion as a signal to collect on this method may be coupled to mass based collection methods for the rapid isolation of compounds.

### RELATED REFERENCES

“Rapid Method to Estimate the Presence of Secondary Metabolites in Microbial”, Higgs, R. E.; Zahn, J. A; Gygi, J. D.; Hilton, M. D.; Appl. Environ. Microbiol. 67:371-376.

“Use of direct-infusion electrospray mass spectrometry to guide empirical development of improved conditions for expression of secondary metabolites from Actinomycetes”, Zahn. J. A.; Higgs, R. E.; Hilton, M. D.; Appl. Envron. Microbiol. 67:377-386.

“A general method for the dereplication of flavonoid glycosides utilizing high performance liquid chromatography mass spectrometric analysis.” Constant, H. L.; Slowing, K.; Graham, J. G.; Pezzuto, J. M.; Cordell, G. A.; Beecher, C. W. W. Phytochemical analysis, 1997, 8:176-180.

Method Information

Gradient column analysis of crude extracts by positive ion mode.

1100 Quaternary Pump 1

| | |
|---|---|
| Control | |
| Column Flow | 1.000 ml/min |
| Stoptime | 4.00 min |
| Posttime | Off |
| Solvents | |
| Solvent A | 98.0% (Water) |
| Solvent B | 0.0% (MeOH) |
| Solvent C | 2.0% (AcCN) |
| Solvent D | 0.0% (iPrOH) |
| PressureLimits | |
| Minimum Pressure | 0 bar |
| Maximum Pressure | 400 bar |
| Auxiliary | |
| Maximal Flow Ramp | 100.00 ml/minˆ 2 |
| Primary Channel | Auto |
| Compressibility | 100*10ˆ -6/bar |
| Minimal Stroke | Auto |
| Store Parameters | |
| Store Ratio A | Yes |
| Store Ratio B | Yes |
| Store Ratio C | Yes |
| Store Ratio D | Yes |
| Store Flow | Yes |
| Store Pressure | Yes |
| Agilent 1100 Contacts Option | |
| Contact 1 | Open |
| Contact 2 | Open |
| Contact 3 | Open |
| Contact 4 | Open |

Timetable

| Time | Solv. B | Solv. C | Solv. D | Flow | Pressure |
|---|---|---|---|---|---|
| 0.00 | 0.0 | 2.0 | 0.0 | 1.000 | |
| 0.01 | 0.0 | 2.0 | 0.0 | | |
| 0.30 | 0.0 | 95.0 | 0.0 | | |
| 1.50 | 0.0 | 95.0 | 0.0 | | |
| 1.60 | 0.0 | 2.0 | 0.0 | | |
| 4.00 | 0.0 | 2.0 | 0.0 | | |

Agilent 1100 Contacts Option Timetable
Timetable is empty
Agilent 1100 Diode Array Detector 1

Signals

| Signal | Store | Signal, Bw | Reference, Bw [nm] |
|---|---|---|---|
| A: | Yes | 215 4 | 450 100 |
| B: | No | 254 4 | 450 100 |
| C: | No | 280 4 | 450 100 |
| D: | No | 250 16 | Off |
| E: | No | 280 16 | Off |

Spectrum

| Store Spectra | Apex + Baselines | |
|---|---|---|
| Range from | 190 | nm |
| Range to | 600 | nm |
| Range step | 2.00 | nm |
| Threshold | 1.00 | mAU |

Time

| | |
|---|---|
| Stoptime | As pump |
| Posttime | Off |

Required Lamps

| | |
|---|---|
| UV lamp required | Yes |
| V is lamp required | Yes |

Autobalance

| | |
|---|---|
| Prerun balancing | Yes |
| Postrun balancing | No |
| Margin for negative Absorbance | 100 mAU |
| Peakwidth | > 0.1 min |
| Slit | 4 nm |

Analog Outputs

| | |
|---|---|
| Zero offset ana. out. 1 | 5% |
| Zero offset ana. out. 2 | 5% |
| Attenuation ana. out. 1 | 1000 mAU |
| Attenuation ana. out. 2 | 1000 mAU |

Mass Spectrometer Detector

General Information

| | |
|---|---|
| Use MSD | Enabled |
| Ionization Mode | APCI |
| Tune File | atunes.tun |
| StopTime | asPump |
| Time Filter | Enabled |
| Data Storage | Condensed |
| Peakwidth | 0.15 min |
| Scan Speed Override | Disabled |

Signals

[Signal 1]

| | |
|---|---|
| Polarity | Positive |
| Fragmentor Ramp | Disabled |

Scan Parameters

| Time | Mass Range | | Frag- | Gain | Thres- | Step- |
|---|---|---|---|---|---|---|
| (min) | Low | High | mentor | EMV | hold | size |
| 0.00 | 110.00 | 1500.00 | 70 | 1.0 | 500 | 0.15 |

[Signal 2]

| | |
|---|---|
| Polarity | Positive |
| Fragmantor Ramp | Disabled |

Scan Parameters

| Time | Mass Range | | Frag- | Gain | Thres- | Step- |
|---|---|---|---|---|---|---|
| (min) | Low | High | mentor | EMV | hold | size |
| 0.00 | 110.00 | 1500.00 | 110 | 1.0 | 500 | 0.15 |

[Signal 3]

Not Active

[Signal 4]

Not Active
Spray Chamber

[MSZones]

| | | |
|---|---|---|
| Gas Temp | 350 C. | maximum 350 C. |
| Vaporizer | 375 C. | maximum 500 C. |
| DryingGas | 3.0 l/min | maximum 13.0 l/min |
| Neb Pres | 60 psig | maximum 60 psig |
| VCap (Positive) | 3000 V | |
| VCap (Negative) | 3000 V | |
| Corona (Positive) | 4.0 μA | |
| Corona (Negative) | 15 μA | |

FIA Series

| | |
|---|---|
| FIA Series in this Method | Disabled |

Time Setting

| | |
|---|---|
| Time between Injections | 1.00 min |

Agilent 1100 Column Thermostat 1

| | |
|---|---|
| Temperature settings | |
| Left temperature | 35.0 ° C. |
| Right temperature | Same as left |
| Enable analysis | When Temp. is within |
| | setpoint +/− 0.8° C. |
| Store left temperature | Yes |
| Store right temperature | No |
| Time | |
| Stoptime | As pump |
| Posttime | Off |
| Column Switching Valve | Column 2 |
| Timetable is empty | |

During the process create a background file by looking for a certain percentage signal occurrence per mass unit. Use the Summary.m program to create this background spectra for use later in step 5 below.

[image]

[image]

[image]

[image]

[image]

[image]

[image]

[image]

[image]

[image]

[image]

[image]

[image]

[image]

[image]

[image]

[image]

[image]

[image]

[image]

[image]

### EXAMPLE 16

### Plasmid DNA Transformation Protocol for Pseudomonas

### a. Preparation of Electroporation Competent Cells

1 ml of overnight culture is innoculated into 100 ml LB, bacteria are incubated in the 30C shaker until OD 600 reading reaches 0.5-0.7. The bacteria are harvested by spinning@3000 rpm for 10 minutes at 4 C.

The resulting cell pellet is washed with 100 ml ice-cold ddH20, spun@3000 rpm for 10 minutes at 4 C to collect the cells. The washing is repeated. The cells are then washed with 50 ml 10% ice-cold glycerol(in ddH20) once and collected by spinning@3000 rpm for 10 minutes at 4C. The bacteria cell is resuspended into 2 ml ice-cold 10% glycerol(in ddH20) 50 ul or 100 ul is aliquoted into each of the tubes and stored at −80 C.

### b. Electroporation

1 ul plasmid DNA is mixed with 50 ul competent cell and kept on ice for 5 minutes. The mixture is transferred to a pre-chilled cuvette(0.2 cm gap, Bio-Rad). The DNA is transformed into bacteria by electroporation with Bio-Rad machine. (Setting: Volts: 2.25KV; time: 5 ms; capacitance: 25 uF)

300 ul SOC medium is added to the cell mixture and bacteria are incubated at 30 C shaker for one hour. A certain amount of culture is spread on LA plate with antibiotics and the plates were incubated at 30 C.

### EXAMPLE 17

### Transformation of Yeast Cells by Electoporation

One day before the experiment, 10 ml of YPD medium is inoculated with a single yeast colony of the strain to be transformed. It is grown overnight to saturation at 30° C. On the day of competent cell preparation, the total volume of yeast overnight culture is transferred to a 2L baffled flask containing 500 ml YPD medium. The culture is grown with vigorous shaking at 30° C. to an OD₆₀₀≅0.8-1.0.

500 ml of culture is harvested by centrifuging at 4000× g, 4° C., for 5 min in autoclaved bottles. The supernatant is subsequently discarded. The cell pellet is washed in 250 ml cold sterile water. Washing is repeated twice. The supernatant is discarded.

The pellet is resuspended in 30 ml of ice-cold 1 M Sorbitol. The suspension is transferred into a sterile 50 ml conical tube. The mixture is centrifuged in a GP-8 centrifuge 2000 rpm, 4° C. for 10 min. The supernatant is discarded.

The pellet is resuspended in 50 μl of ice-cold 1M Sorbitol. The final volume of resuspended yeast should be 1.0 to 1.5 ml and the final OD600 should be ˜200.

In a sterile, ice-cold 1.5-ml microcentrifuge tube, 40 ul concentrated yeast cells are mixed with lug of DNA contained in >5 μl. The mixture is transferred to an ice-cold 0.2-cm-gap disposable electroporation cuvette and pulsed at 1.5 kV, 25 uF, 200 Ω. It should be noted that the time constant reported by the Gene Pulser will vary from 4.2 to 4.9 msec. Times <4 msec or the presence of a current arc (evidenced by a spark and smoke) indicate that the conductance of the yeast/DNA mixture is too high. 400 μl ice-cold 1M sorbitol is added to the cuvette and the yeast is recovered, with gentle mixing. 200 μl aliquots of the east suspension should be spread directly on sorbitol selection plates. Incubate 3 to 6 days at 30° C. until colonies appear.

### LITERATURE CITED

1. Gibbs, J. B., Mechanism-Based Target Identification and Drug Discovery in Cancer Research. Science 2000, 287, 1969-73

2. Garret, M. D., Workman, P. Discovering Novel Chemotherapeutic Drugs for the Third Millennium. Eur. J. Cancer 1999, 35, 2010-30

3. Hanahan, D., Weinberg, R. A., The Hallmarks of Cancer. Cell 2000, 100, 57-70

4. Druker, B. J., Nicholas, B. L., Lessons learned from the development of an Abl tyrosine kinase inhibitor for chronic myelogenous leukemia. J. Clin. Invest. 2000, 105, 3-7

5. Sikic, B. I., New Approaches in cancer treatment. Ann. Onc. 1999,10, S149-S153

6. Gibbs, J. B., Anticancer drug targets: growth factors and growth factor signaling. J. Clin. Invest. 2000, 105, 9-13

7. Drews, J., Drug Discovery: A historical perspective. Science 2000, 287, 1960-64

8. Harvey, A. L., Medicines from nature: are natural products still relevant to drug discovery? Trends Pharmacol. Sci. 1999, 20, 196-197

9. Cragg, G. M., Newman, D. J., Snader, K. M. Natural products in drug discovery and development. J. Nat. Prod. 1997, 60, 52-60

10. Verdine, G. L., The combinatorial chemistry of nature. Nature 1996, 384, 11-13

11. Demain, A. L., and J. E. Davies. Manual of industrial Microbiology and biotechnology; ASM Press: Washington D.C., 1999

12. Mc Daniel, R., et al., Rational design of aromatic polyketide natural products by recombinant assembly of enzymatic subunits. Nature 1995, 375, 549-554

13. Jacobsen, J. R., D. E. Cane, and C. Khosla, Spontaneous priming of a downstream module in 6-deoxyerythronolide B synthase leads to polyketide biosynthesis. Biochem. 1998, 37, 4928-4934

14. Donadio, S., McAlpine, J. B., Sheldon, P. J., Jackson, M., and Katz, L., An erythromycin analog produced by reprogramming of polyketide synthesis.Proc. Natl. Acad. Sci. U.S.A. 1993, 90, 7119-23

15. Cortes, J. et al, Science, Repositioning of a domain in a modular polyketide synthase to promote specific chain cleavage 1995, 268, 1487-89

16. Amann, R. I. L. W., Schleifer K. H., Phylogenetic identification and in situ detection of individual microbial cells without cultivation. Microbiol. Rev. 1995, 59, 143-169

17. Robertson, D. E., et al. The discovery of new biocatalysts from microbial diversity. SIM News 1996, 46, 3-8

18. Stein, J. L., et al., Characterization of uncultivated prokaryotes: isolation and analysis of a 40-kilobase-pair genome fragment from a planktonic marine Archaeon. J. Bacteriol. 1996, 178, 591-599

19. Short, J. M., Recombinant approaches for accessing biodiversity. Nat. Biotechnol. 1997, 15, 1322-23

20. Sundberg, S. A., High-throughput and ultra-high-throughout screening: solution- and cell-based approaches. Curr. Opin. Biotech. 2000, 11, 47-53

21. Alvi, K. A., Pu, H., Asterriquinones produced by Aspergillus candidus inhibit binding of the Grb-2 adapter to phosphorylated EGF receptor tyrosine kinase. J. Antibiotics 1999, 52, 215-223

22. Levitzki, A., Gazit, A., Tyrosine Kinase inhibition: an approach to drug development. Science 1995, 267, 1782-88

23. Alberts, B., Bray, D., Lewis, J., Raff, M., Roberts, K., and J. D. Watson, Molecular biology of the cell; Garland Publishing, Inc.: New York, 1994

24. Kolibaba, K. S., Druker, B. J., Protein tyrosine kinases and cancer. Biochim Biophysica Acta 1997, 1333, F217-F248

25. Neal, D. E., Sharples, L., Smith, K., Fennelly, J., Hall, R. R., Harris, A. L., The epidermal growth factor receptor and the prognosis of bladder cancer. Cancer 1990, 65, 1619-25

26. Nicholson, S., Richard, J., Sainsbury, C., Halcrow, P., Kelly, P., Angus, B., Wright, C., Henry, J., Farndon, J., Harris, A., Epidermal growth factor receptor (EGFr) status associated with failure of primary endocrine therapy in elderly postmenopausal patients with breast cancer. Br. J. Cancer 1991, 63, 146-150

27. Klijn, J. G. M., Bems, P. M. J. J., Schmitz, P. I. M., Foekens, J. A., The clinical significance of epidermal growth factor receptor (EGF-R) in human breast cancer: a review on 5232 patients. Endocr. Rev. 1992, 12, 3-17

28. Hiesiger, E., Hayes, R., Pierz, D., Budzilovich, G., Prognostic relevance of epidermal growth factor receptor (EGF-R) and c-neu/erbB2 expression in glioblastomas (GBMs). Neurooncol. 1993, 16, 93-104

29. Tateishi, M., Ishida, T., Mitsudomi, T., Kaneko, S., Sugimachi, K., Immunohistochemical evidence of autocrine growth factors in adenocarcinoma of the human lung Cancer Res. 1990, 50, 7077-80

30. Gorgoulis, V., Aninos, D., Mikou, P., Kanavaros, P., Karameris, A., Joardanoglu, J., Rasidakis, A., Veslemes, M., Ozanne, B., Spandidos, D. A., Expression of EGF, TGF-alpha and EGFR in squamous cell lung carcinomas Anticancer Res. 1992, 12, 1183-87

31. Sharif, T. R., Sharif, M., A high throughput system for the evaluation of protein kinase C inhibitors based on Elk1 transcriptional activation in human astrocytoma cells. Int. J. Onc. 1999, 14, 327-335

32. Li, Q., Vaingankar, S. M., Green, H. M., Green, M. M., Activation of the 9E3/cCAF chemokine by phorbol esters occurs via multiple signal transduction pathways that converge to MEK1/ERK2 and activate the Elk1 transcription factor. J Biol Chem 1999, 274, 15454

33. Treisman, R., Regulation of transcription by MAP kinase cascades. Curr. Opin. Cell Biol. 1996, 8, 205-215

34. Engler, D. A., Matsunami, R. K., Campion, S. R., Stringer, C. D., Stevens, A., Niyogi, S., Cloning of authentic human epidermal growth factor as a bacterial secretory protein and its initial structure-function analysis by site-directed mutagenesis. J. Biol. Chem. 1988, 263, 12384-390

35. Salmelin, C., Hovinen, J., Vilpo, J., Polymyxin permeabilization as a tool to investigate cytotoxicity of therapeutic aromatic alkylators in DNA repair-deficient Escherichia coli strains. Mut. Res. 2000, 467, 129-138

36. Gray, F., Kenney, J. S., Dunne, J. F., Secretion capture and report web: use of affinity derivatized agarose microdroplets for the selection of hybridoma cells. J. Immunol. Methods 1995, 182, 155-163

37. Powell, K. T., Weaver, J. C., Gel microdroplets and flow cytometry: rapid determination of antibody secretion by individual cells within a cell population. Bio/Technology 1990, 8, 333-337

38. Jan van der Wal, F., Luirink, J., Oudega, B., Bacteriocin release proteins: made of action, structure, and biotechnological application. FEMS Biol. Rev 1995, 17, 381-

39. Majno, G., Joris, I., Apoptosis, oncosis, and necrosis: an overview of cell death. Am. J. Pathol. 1995, 146, 3-15

40. Wyllie, A. H., Kerr, J. F. R., Currie, A. R., Cell death; the significance of apoptosis. Int. Rev. Cytol. 1980, 68, 251-356

41. Sikic, B. I., Rozencweig, M., Carter, S. K., Eds. Bleomycin chemotherapy; Academic Press: Orlando, FL, 1985

42. Deng, J L., Newman, D. J., Hecht, S. M., Use of COMPARE analysis to discover functional analogues of bleomycin. J. Nat. Prod. 2000, 63, 1269-72

43. Ortiz, L. A., Moroz, K., Liu, J Y., Hoyle, G. W., Hammond, T., Hamilton, R., Holian, A., Banks, W., Brody, A. R., Friedman, M., Alveolar macrophage apoptosis and TNF-a, but not p53, expression correlate with murine, response to bleomycin. Am. J. Physiol. 1998, 275, L1208-L1218

44. Kumagai, T., Sugiyama, M., Protection of mammalian cells from the toxicity of bleomycin by expression of a bleomycin-binding protein gene from streptomyces verticillus. J. Biochem. 1998, 124, 835-841

45. Benitez-Bribiesca, L., Sanchez-Suarez, P., Oxidative damage, bleomycin, and gamma radiation induce different types of DNA strand breaks in normal lymphocytes and thymocytes. Ann. NY Academy Sci. 1999, 887, 133-149

46. Du, L., Sanchez, C., Chen, M., Edwards, D. J., Shen, B., The biosynthetic gene cluster for the antitumor drug bleomycin from Streptomyces verticillus ATCC 15003 supporting functional interactions between nonribosomal peptide synthetases and a polyketide synthase. Chem. & Biol. 2000, 7, 623-642

49. Guiseley, K. B. U.S. Pat. No. 3,956,273, Modified Agarose and Agar and Methods of Making Same. May 11, 1976.

50. Phospholipids Handbook; Cevc, G., Ed.; Marcel Dekker: New York, 1993.

51. Ringsdorf, H.; Schlarb, B.; Venzmer, J. Molecular Architecture and Function of Polymeric Oriented Systems: Models for Study of Organization, Surface Recognition, and Dynamics of Biomembranes. Angew. Chem., Int. Ed. Engl. 1988, 27, 113-158 and references cited therein.

52. O'Brien, D. F.; Ramaswami, V. Polymerized Vesicles. Encycl. Polym. Sci. Eng. 1989,17,108-135.

53. Nilsson, K.; Brodelius, P.; Mosbach, K. Entrapment of Microbial and Plant Cells in Beaded Polymers. Methods in Emzymology, 1987, 135, 222-230 and references cited therein.

54. Kroger, N.; Deutzmann, R.; Sumper, M. Polycationic Peptides from Diatom Biosilica That Direct Silica Nanosphere Formation. Science 1999, 286, 1129-1132.

55. Cha, J. N.; Stucky, G. D.; Morse, D. E.; Deming, T. J. Biomimetic Synthesis of Ordered Silica Structures Mediated by Block Copolypeptides. Nature 2000, 403, 289 292.

56. Bukanov, N. O., Demidov, V. V., Nielsen, P. E. & Frank-Kamenetskii, M. D. (1998). PD-loop: A complex of duplex DNA with an oligonucleotide. PNAS, 95 (10), 5516-5520.

57. Brenner, S., Williams, S. R., Vermaas, E. H., Storck, T., Moon, K., McCollum, C., Mao, J., Luo, S., Kirchner, J. J., Eletr, S., DuBridge, R. B., Burcham, T. & Albrecht, G. (1999). In vitro cloning of complex mixtures of DNA on microbeads: Physical separation of differentially expressed cDNAs. PNAS, 97 (4), 1665-1670.

58. Goryshin, I. Y., & Reznikoff, W. S. (1998). Tn5 in vitro transposition. J. Biol. Chem., 273, 7367-7374.

59. Jayasena, V. K. & Johnston, B. H. (1993). Complement-stabilized D-loop: RecA-catalyzed stable pairing of linear DNA molecules at internal sites. J. Mol. Biol., 230, 1015-1024.

60. Lohse, J., Dahl, 0. & Nielsen, P. E. (1999). Double duplex invasion by peptide nucleic acid: A general principle for sequence-specific targeting of double-stranded DNA. PNAS, 96 (21), 11804-11808.

61. Sena, E. P. & Zarling, D. A. (1993). Targeting in linear DNA duplexes with two complementary probe strands for hybrid stability. Nature Genetics

While the invention has been described in detail with reference to certain preferred embodiments thereof, it will be understood that modifications and variations are within the spirit and scope of that which is described and claimed.

What is claimed is:

1. A method for identifying a polynucleotide in a liquid phase comprising:
a) contacting a plurality of polynucleotides derived from at least one organism with at least one nucleic acid probe under conditions that allow hybridization of the probe to the polynucleotides having complementary sequences, wherein the probe is labeled with a detectable molecule; and
b) identifying a polynucleotide of interest with an analyzer that detects the detectable molecule.

2. The method of claim 1, wherein the polynucleotides are from a mixed population of cells.

3. The method of claim 1, wherein the polynucleotides are in a library.

4. The method of claim 3 wherein the library is an expression library.

5. The method of claim 3 wherein the library is an environmental expression library.

6. The method of claim 1, wherein the nucleic acid probe is from at least about 15 bases to about 100 bases.

7. The method of claim 1, wherein the nucleic acid probe is from at least about 100 bases to about 500 bases.

8. The method of claim 1, wherein the nucleic acid probe is from at least about 500 bases to about 1,000 bases.

9. The method of claim 1, wherein the nucleic acid probe is from at least about 1,000 bases to about 5,000 bases.

10. The method of claim 1, wherein the nucleic acid probe is from at least about 5,000 bases to about 10,000 bases.

11. The method of claim 1, wherein the detectable molecule is a fluorescent molecule.

12. The method of claim 1, wherein the detectable molecule is a magnetic molecule.

13. The method of claim 1, wherein the detectable molecule modulates a magnetic field.

14. The method of claim 1, wherein the detectable molecule modulates the dielectric signature of the clone.

15. The method of claim 1, wherein the analyzer is a FACS analyzer.

16. The method of claim 1, wherein the analyzer is a magnetic field sensing device.

17. The method of claim 9, wherein the magnetic field sensing device is a Super Conducting Quantum Interference Device.

18. The method of claim 1, wherein the analyzer is a multipole coupling spectroscopy device.

19. The method of claim 1, wherein the organism is from an environmental sample.

20. The method of claim-1, wherein the environmental sample is selected from the group consisting of: geothermal fields, hydrothermal fields, acidic soils, sulfotara mud pots, boiling mud pots, pools, hot-springs, geysers, marine actinomycetes, metazoan, endosymbionts, ectosymbionts, tropical soil, temperate soil, arid soil, compost piles, manure piles, marine sediments, freshwater sediments, water concentrates, hypersaline sea ice, super-cooled sea ice, arctic tundra, Sargosso sea, open ocean pelagic, marine snow, microbial mats, whale falls, springs, hydrothermal vents, insect and nematode gut microbial communities, plant endophytes, epiphytic water samples, industrial sites and ex situ enrichments.

21. The method of claim-2, wherein the environmental sample is selected from the group consisting of: eukaryotes, prokaryotes, myxobacteria (epothilone), air, water, sediment, soil or rock.

22. The method of claim 1, wherein the organism comprises a microorganism.

23. The method of claim 19, wherein the environmental sample contains extremophiles.

24. The method of claim 23, wherein the extremophiles are selected from the group consisting of hyperthermophiles, psychrophiles, halophiles, psychrotrophs, alkalophiles, and acidophiles.

25. The method of claim 1, further comprising encapsulation of the polynucleotide in a microenvironment.

26. The method of claim 25, wherein the microenvironment is selected from beads, high temperature agaroses, gel microdroplets, cells, ghost red blood cells, macrophages, or liposomes.

27. The method of claim 26, wherein the microenvironment is a gel microdroplet.

28. The method of claim 25, wherein the detectable molecule is a biotinylated substrate.

29. The method of claim 28, wherein the biotinylated substrate comprises a core fluorophore structure, a spacer connected to the fluorophore structure by a first connector and connected to the bioactivity or biomolecume of interest by a second connector, and two functional groups, wherein each functional group is attached to the fluorophore structure by a connector unit.

30. The method of claim 29, wherein the fluorophore is selected from the group consisting of coumarins, resorufins and xanthenes.

31. The method of claim 29, wherein the spacer is selected from the group consisting of:
alkanes, and oligoethyleneglycols.

32. The method of claim 29, wherein the connector units are selected from the groups consisting of ether, amine, amide, ester, urea, thiourea and other moieties.

33. The method of claim 29, wherein the functional groups are independently selected from the group consisting of straight alkanes, branched alkanes, monosaccharides, oligosaccharides, unsaturated hydrocarbons and aromatic groups.

34. The method of claim 25, wherein the analyzer is a flow cytometer.

35. The method of claim 28, wherein the biotinylated substrate comprises a core fluorophore structure, a spacer connected to the fluorophore structure by a first connector and connected to the bioactivity or biomolecule of interest by a second connector, and a quencher component, attached to the cluorophore by a polymer.

36. The method of claim 35, wherein the fluorophore is selected from the group consisting of acridines, coumarins, fluorescein, rhodamine, BOPIDY, resorufin, and porphyrins.

37. The method of claim 35, wherein the quencher is a moiety capable of quenching fluorescence of the fluorophore.

38. The method of claim 35, wherein the polymer is selected from the group consisting of amines, ethers, esters, amides, peptides and oligosaccharides.

39. The method of claim 35, wherein the spacer is selected from the group consisting of:
alkanes, and oligoethyleneglycols.

40. The method of claim 35, wherein the first and second connectors are selected from the groups consisting of ether, amine, amide, ester, urea, thiourea and other moieties.

41. The method of claim 1, wherein the polynucleotide of interest encodes an enzyme.

42. The method of claim 41, wherein the enzyme is selected from the group consisting of lipases, esterases, proteases, glycosidases, glycosyl transferases, phosphatases, kinases, mono- and dioxygenases, haloperoxidases, lignin peroxidases, diarylpropane peroxidases, eposize hydrolases, nitrile hydratases, nitrilases, transaminases, amidases, and acylases.

43. The method of claim 1, wherein the polynucleotide of interest encodes a small molecule.

44. The method of claim 1, wherein the polynucleotide of interest, or fragments thereof, comprise one or more operons, or portions thereof.

45. The method of claim 44, wherein the operons, or portions thereof, encodes a complete or partial metabolic pathway.

46. The method of claim 44, wherein the operons or portions thereof encoding a complete or partial metabolic pathway encodes polyketide syntheses.

47. A method for identifying a polynucleotide encoding a polypeptide of interest comprising:
co-encapsulating in a microenvironment a plurality of library clones containing DNA obtained from a mixed population of organisms, with a mixture of oligonucleotide probes comprising a detectable label and at least a portion of a polynucleotide sequence encoding a polypeptide of interest having a specified bioactivity under such conditions and for such time as to allow interaction of complementary sequences; and
identifying clones containing a complement to the oligonucleotide probe encoding the polypeptide of interest by separating clones with an analyzer that detects the detectable label.

48. A method for high throughput screening of a polynucleotide library for a polynucleotide of interest that encodes a molecule of interest, comprising:
(a) contacting a library containing a plurality of clones comprising polynucleotides derived from a mixed population of organisms with a plurality of oligonucleotide probes labeled with a detectable molecule; and
(b) separating clones with an analyzer that detect the detectable molecule.

49. The method of claim 48, further comprising:
(a) contacting the separated clones with a reporter system that identifies a polynucleotide encoding the molecule of interest; and
(b) identifying clones capable of modulating expression or activity of the reporter system thereby identifying a polynucleotide of interest.

50. The method of claim 48, wherein the library is an expression library.

51. The method of claim 48, wherein the mixed population of organisms is from an environmental sample.

52. The method of claim 51, wherein the environmental sample is selected from the group consisting of: geothermal fields, hydrothermal fields, acidic soils, sulfotara mud pots, boiling mud pots, pools, hot-springs, geysers, marine actinomycetes, metazoan, endosymbionts, ectosymbionts, tropical soil, temperate soil, arid soil, compost piles, manure piles, marine sediments, freshwater sediments, water concentrates, hypersaline sea ice, super-cooled sea ice, arctic tundra, Sargosso sea, open ocean pelagic, marine snow, microbial mats, whale falls, springs, hydrothermal vents, insect and nematode gut microbial communities, plant endophytes, epiphytic water samples, industrial sites and ex situ enrichments.

53. The method of claim 51, wherein the environmental sample is selected from the group consisting of: eukaryotes, prokaryotes, myxobacteria (epothilone), air, water, sediment, soil or rock.

54. The method of claim 48, wherein the mixed population of organisms comprises microorganisms.

55. The method of claim 51, wherein the environmental sample contains extremophiles.

56. The method of claim 55, wherein the extremophiles are selected from the group consisting of hyperthermophiles, psychrophiles, halophiles, psychrotrophs, alkalophiles, and acidophiles.

57. The method of claim 49, wherein the reporter system is a bioactive substrate.

58. The method of claim 57, wherein the bioactive substrate comprises C12FDG.

59. The method of claim 58, wherein the bioactive substrate further comprises a lipophilic tail.

60. The method of claim 49, further comprising prior to (a):
(i) obtaining polynucleotides from a mixed population of organisms; and
(ii) generating a polynucleotide library.

61. The method of claim 60, further comprising normalizing the polynucleotides prior to generating the library.

62. The method of claim 48, further comprising encapsulation of the clones in a gel microdrop.

63. The method of claim 62, wherein the detectable molecule is a biotinylated substrate.

64. The method of claim 63, wherein the biotinylated substrate comprises a core fluorophore structure, a spacer connected to the fluorophore structure by a first connector and connected to the bioactivity or biomolecume of interest by a second connector, and two functional groups, wherein each functional group is attached to the fluorophore structure by a connector unit.

65. The method of claim 64, wherein the fluorophore is selected from the group consisting of coumarins, resorufins and xanthenes.

66. The method of claim 64, wherein the spacer is selected from the group consisting of:
alkanes, and oligoethyleneglycols.

67. The method of claim 64, wherein the connector units are selected from the groups consisting of ether, amine, amide, ester, urea, thiourea and other moieties.

68. The method of claim 64, wherein the functional groups are independently selected from the group consisting of straight alkanes, branched alkanes, monosaccharides, oligosaccharides, unsaturated hydrocarbons and aromatic groups.

69. The method of claim 62, wherein the analyzer is a flow cytometer.

70. The method of claim 63, wherein the biotinylated substrate comprises a core fluorophore structure, a spacer connected to the fluorophore structure by a first connector and connected to the bio activity or biomolecule of interest by a second connector, and a quencher component, attached to the cluorophore by a polymer.

71. The method of claim 70, wherein the fluorophore is selected from the group consisting of acridines, coumarins, fluorescein, rhodamine, BOPIDY, resorufin, and porphyrins.

72. The method of claim 70, wherein the quencher is a moiety capable of quenching fluorescence of the fluorophore.

73. The method of claim 70, wherein the polymer is selected from the group consisting of amines, ethers, esters, amides, peptides and oligosaccharides.

74. The method of claim 70, wherein the spacer is selected from the group consisting of:
alkanes, and oligoethyleneglycols.

75. The method of claim 70, wherein the first and second connectors are selected from the groups consisting of ether, amine, amide, ester, urea, thiourea and other moieties.

76. The method of claim 48, wherein the polynucleotide of interest encodes an enzyme.

77. The method of claim 76, wherein the enzyme is selected from the group consisting of lipases, esterases, proteases, glycosidases, glycosyl transferases, phosphatases, kinases, mono- and dioxygenases, haloperoxidases, lignin peroxidases, diarylpropane peroxidases, eposize hydrolases, nitrile hydratases, nitrilases, transaminases, amidases, and acylases.

78. The method of claim 49, wherein the reporter system comprises a detectable label.

79. The method of claim 49, wherein the reporter system comprises a first test protein linked to a DNA binding moiety and a second test protein linked to a transcriptional activation moiety, wherein modulation of the interaction of the first test protein linked to a DNA binding moiety with the second test protein linked to a transcription activation moiety results in a change in the expression of a detectable protein.

80. The method of claim 49, wherein the polynucleotide of interest encodes a small molecule.

81. The method of claim 49, wherein the polynucleotide of interest, or fragments thereof, comprise one or more operons, or portions thereof.

82. The method of claim 81, wherein the operons, or portions thereof, encodes a complete or partial metabolic pathway.

83. The method of claim 82, wherein the operons or portions thereof encoding a complete or partial metabolic pathway encodes polyketide syntheses.

84. The method of claim 48, wherein the analyzer is a fluorescence activated cell sorting (FACS) apparatus.

85. The method of claim 48, wherein the analyzer is a magnetic field sensing device.

86. The method of claim 85, wherein the magnetic field sensing device is a Super Conducting Quantum Interference Device.

87. The method of claim 48, wherein the analyzer is a multipole coupling spectroscopy device.

88. The method of claim 48, wherein the plurality of oligonucleotide probes have different nucleic acid sequences.

89. The method of claim 88, wherein the sequences are portions of a polynucleotide encoding a molecule of interest.

90. The method of claim 48, wherein the plurality of oligonucleotide probes have the same nucleic acid sequence.

91. A method of screening for a polynucleotide encoding an activity of interest, comprising:
(a) obtaining polynucleotides from an environmental sample;
(b) normalizing the polynucleotides obtained from the sample;
(c) generating a library from the normalized polynucleotides;
(d) contacting the library with a plurality of oligonucleotide probes comprising a detectable label and at least a portion of a polynucleotide sequence encoding a polypeptide of interest having a specified activity to select library clones positive for a sequence of interest; and
(e) selecting clones with an analyzer that detects the detectable label.

92. The method of claim 91, further comprising:
(a) contacting the selected clones with a reporter system that identifies a polynucleotide encoding the activity of interest; and
(b) identifying clones capable of modulating expression or activity of the reporter system thereby identifying a polynucleotide of interest; wherein the positive clones contain a polynucleotide sequence encoding an activity of interest which is capable of catalyzing the bioactive substrate.

93. A method for screening polynucleotides, comprising:
(a) contacting a library of polynucleotides wherein the polynucleotides are derived from a mixed population of organism with a probe oligonucleotide labeled with a fluorescence molecule, which fluoresce upon binding of the probe to a target polynucleotide of the library, to select library polynucleotides positive for a sequence of interest;
(b) separating library members that are positive for the sequence of interest with a fluorescent analyzer that detects fluorescence; and
(c) expressing the selected polynucleotides to obtain polypeptides.

94. The method of claim 93, further comprising:
(a) contacting the polypeptides with a reporter system; and
(b) identifying polynucleotides encoding polypeptides capable of modulating expression or activity of the reporter system.

95. A method for obtaining an organism from a mixed population of organisms in a sample comprising:
(a) encapsulating in a microenvironment at least one organism from the sample;
(b) incubating the encapsulated at least one organism under such conditions and for such a time to allow the at least one microorganism to grow or proliferate; and
(c) sorting the encapsulated at least one organism by a flow cytometer to obtain an organism from the sample.

96. The method of claim 95, wherein the mixed population of organisms is from an environmental sample.

97. The method of claim 96, wherein the environmental sample is selected from the group consisting of: geothermal fields, hydrothermal fields, acidic soils, sulfotara mud pots, boiling mud pots, pools, hot-springs, geysers, marine actinomycetes, metazoan, endosymbionts, ectosymbionts, tropical soil, temperate soil, arid soil, compost piles, manure piles, marine sediments, freshwater sediments, water concentrates, hypersaline sea ice, super-cooled sea ice, arctic tundra, Sargosso sea, open ocean pelagic, marine snow, microbial mats, whale falls, springs, hydrothermal vents, insect and nematode gut microbial communities, plant endophytes, epiphytic water samples, industrial sites and ex situ enrichments.

98. The method of claim 96, wherein the environmental sample is selected from the group consisting of: eukaryotes, prokaryotes, myxobacteria (epothilone), air, water, sediment, soil or rock.

99. The method of claim 95, wherein the mixed population of organisms comprises microorganisms.

100. The method of claim 96, wherein the environmental sample contains extremophiles.

101. The method of claim 101, wherein the extremophiles are selected from the group consisting of hyperthermophiles, psychrophiles, halophiles, psychrotrophs, alkalophiles, and acidophiles.

102. The method of claim 95, wherein the flow cytometer comprises a magnetic field sensing device.

103. The method of claim 102, wherein the magnetic field sensing device is a Super Conducting Quantum Interference Device.

104. The method of claim 95, wherein the flow cytometer is a multipole coupling spectroscopy device.

105. A method for identifying a bioactivity or biomolecule of interest, comprising:
(a) transferring a library containing a plurality of clones comprising polynucleotides derived from a mixed population of organisms or more than one organism, to a bacterial host cell;
(b) contacting the bacterial host cell with a mammalian host cell containing a detectable reporter molecule in a microenvironment; and
(c) separating clones with an analyzer that detects the detectable molecule.

106. The method of claim 105, wherein the microenvironment is selected from beads, high temperature agaroses, gel microdroplets, cells, ghost red blood cells, macrophages, or liposomes.

107. The method of claim 106, wherein the liposomes are prepared from one or more phospholipids, glycolipids, steroids, alkyl phosphates or fatty acid esters.

108. The method of claim 107, wherein the phospholipids are selected from the group consisting of lecithin, sphingomyelin and dipalmitoyl.

109. The method of claim 107, wherein the steroids are selected from the group consisting of cholesterol, cholestanol and lanosterol.

110. The method of claim 105, wherein the detectable reporter contains a bioluminescent molecule, a chemiluminescent molecule, a colorimetric molecule, an electromagnetic molecule, an isotopic molecule, a thermal molecule or an enzymatic substrate.

111. The method of claim 110, wherein the bioluminescent molecule is green fluorescent protein (GFP) or red fluorescent protein (RFP).

112. The method of claim 105, wherein the analyzer is a FACS analyzer.

113. The method of claim 105, wherein the analyzer is capillary-based screening apparatus.

114. A method for identifying a bioactivity or biomolecule of interest, comprising:
(a) transferring a library containing a plurality of clones comprising polynucleotides derived from a mixed population of organisms or more than one organism, to a first host cell;
(b) contacting the first host cell with a second host cell containing a detectable reporter molecule in a microenvironment, wherein the first host cell is different from the second host cell; and
(c) separating clones with an analyzer that detects the detectable molecule.

115. The method of claim 114, wherein the first host cell is a prokaryotic cell.

116. The method of claim 114, wherein the first host cell is a eukaryotic cell.

117. The method of claim 114, wherein the second host cell is a prokaryotic cell.

118. The method of claim 114, wherein the second host cell is a eukaryotic cell.

119. The method of claims 115 or 117, wherein the prokaryotic cell is a bacterial cell.

120. The method of claims 116 or 118, wherein the eukaryotic cell is a mammalian cell.

121. The method of claim 114, wherein the microenvironment is selected from beads, high temperature agaroses, gel microdroplets, cells, ghost red blood cells, macrophages, or liposomes.

122. The method of claim 121, wherein the liposomes are prepared from one or more phospholipids, glycolipids, steroids, alkyl phosphates or fatty acid esters.

123. The method of claim 122, wherein the phospholipids are selected from the group consisting of lecithin, sphingomyelin and dipalmitoyl.

124. The method of claim 122, wherein the steroids are selected from the group consisting of cholesterol, cholestanol and lanosterol.

125. The method of claim 114, wherein the detectable reporter contains a bioluminescent molecule, a chemiluminescent molecule, a colorimetric molecule, an electromagnetic molecule, an isotopic molecule, a thermal molecule or an enzymatic substrate.

126. The method of claim 125, wherein the bioluminescent molecule is green fluorescent protein (GFP) or red fluorescent protein (RFP).

127. The method of claim 114, wherein the analyzer is a FACS analyzer.

128. The method of claim 114, wherein the analyzer is capillary-based screening apparatus.

129. A method for identifying a bioactivity or biomolecule of interest, comprising:
(a) transferring a library containing a plurality of clones comprising polynucleotides derived from a mixed population of organisms or more than one organism, to a host cell; and
(b) contacting the first host cell with a second host cell containing a detectable reporter molecule in a microenvironment, wherein the first host cell and second host cell are different.

130. The method of claim 129, wherein the first host cell is a prokaryotic cell.

131. The method of claim 129, wherein the first host cell is a eukaryotic cell.

132. The method of claim 129, wherein the second host cell is a prokaryotic cell.

133. The method of claim 129, wherein the second host cell is a eukaryotic cell.

134. The method of claims 130 or 132, wherein the prokaryotic cell is a bacterial cell.

135. The method of claims 131 or 133, wherein the eukaryotic cell is a mammalian cell.

136. The method of claim 129, wherein the microenvironment is selected from beads, high temperature agaroses, gel microdroplets, cells, ghost red blood cells, macrophages, or liposomes.

137. The method of claim 136, wherein the liposomes are prepared from one or more phospholipids, glycolipids, steroids, alkyl phosphates or fatty acid esters.

138. The method of claim 137, wherein the phospholipids are selected from the group consisting of lecithin, sphingomyelin and dipalmitoyl.

139. The method of claim 137, wherein the steroids are selected from the group consisting of cholesterol, cholestanol and lanosterol.

140. The method of claim 129, wherein the detectable reporter contains a bioluminescent molecule, a chemiluminescent molecule, a calorimetric molecule, an electromagnetic molecule, an isotopic molecule, a thermal molecule or an enzymatic substrate.

141. The method of claim 140, wherein the bioluminescent molecule is green fluorescent protein (GFP) or red fluorescent protein (RFP).

142. The method of claim 129, further comprising separating the clones with an analyzer that detects the detectable molecule.

143. The method of claim 142, wherein the analyzer is a FACS analyzer.

144. The method of claim 142, wherein the analyzer is a capillary-based screening apparatus.

145. The method of claim 142, wherein the analyzer is a mass spectroscopic screening apparatus.

146. A method for identifying a bioactivity or biomolecule of interest, comprising:
(a) transferring the extract of a library containing a plurality of clones comprising polynucleotides derived from a mixed population of organisms or more than one organism, to a first host cell; and
(b) contacting the extract with a second host cell containing a detectable reporter molecule.

147. The method of claim 146, wherein the first host cell is a prokaryotic cell.

148. The method of claim 146, wherein the first host cell is a eukaryotic cell.

149. The method of claim 146, wherein the second host cell is a prokaryotic cell.

150. The method of claim 146, wherein the second host cell is a eukaryotic cell.

151. The method of claims 147 or 149, wherein the prokaryotic cell is a bacterial cell.

152. The method of claims 148 or 150, wherein the eukaryotic cell is a mammalian cell.

153. The method of claim 146, wherein the extract is contacted with a host cell in a microenvironment.

154. The method of claim 153, wherein the microenvironment is selected from beads, high temperature agaroses, gel microdroplets, cells, ghost red blood cells, macrophages, or liposomes.

155. The method of claim 154, wherein the liposomes are prepared from one or more phospholipids, glycolipids, steroids, alkyl phosphates or fatty acid esters.

156. The method of claim 155, wherein the phospholipids are selected from the group consisting of lecithin, sphingomyelin and dipalmitoyl.

157. The method of claim 155, wherein the steroids are selected from the group consisting of cholesterol, cholestanol and lanosterol.

158. The method of claim 146, wherein the detectable reporter contains a bioluminescent molecule, a chemiluminescent molecule, a calorimetric molecule, an electromagnetic molecule, an isotopic molecule, a thermal molecule or an enzymatic substrate.

159. The method of claim 158, wherein the bioluminescent molecule is green fluorescent protein (GFP) or red fluorescent protein (RFP).

160. The method of claim 146, further comprising separating the clones with an analyzer that detects the detectable molecule.

161. The method of claim 160, wherein the analyzer is a FACS analyzer.

162. The method of claim 160, wherein the analyzer is a capillary-based screening apparatus.

163. The method of claim 160, wherein the analyzer is a mass spectroscopic screening apparatus.

164. A method for identifying a bioactivity or biomolecule of interest, comprising:
a) running the extract of a library containing a plurality of clones comprising polynucleotides derived from a mixed population of organisms or more than one organism, through a column;
b) transferring the extract to a first host cell;
c) contacting the extract with a second host cell containing a detectable reporter molecule; and
d) measuring the mass spectra of the host cell with the extract, wherein a difference in the mass spectra of the host cell with the extract from the mass spectra without the extract is indicative of the presence of a bioactivity or biomolecule of interest in the extract of the library.

165. A sample screening apparatus, comprising:
a plurality of capillaries held together in an array, wherein each capillary comprises at least one wall defining a lumen for retaining a sample;
interstitial material disposed between adjacent capillaries in the array; and
one or more reference indicia formed within of the interstitial material.

166. The apparatus of claim 165, wherein each capillary has an aspect ratio of between 10:1 and 1000:1.

167. The apparatus of claim 166, wherein each capillary has an aspect ratio of between 20:1 and 100:1.

168. The apparatus of claim 165, wherein each capillary has an aspect ratio of between 40:1 and 50:1.

169. The apparatus of claim 165, wherein each capillary has a length of between 5 mm and 10 cm.

170. The apparatus of claim 165, wherein the lumen of each capillary has an internal diameter of between 3 μm and 500 μm.

171. The apparatus of claim 165, wherein the lumen of each capillary has an internal diameter of between 10 μm and 500 μm.

172. The apparatus of claim 165, wherein the plurality of capillaries are fused together to form the array.

173. The apparatus of claim 165, wherein the reference indicia are formed at intervals of a number of capillaries.

174. The apparatus of claim 165, wherein the reference indicia are formed at edges of the array.

175. The apparatus of claim 165, wherein the reference indicia are formed of glass.

176. A capillary for screening a sample, wherein the capillary is adapted for being held in an array of capillaries, the capillary comprising:
a first wall defining a lumen for retaining the sample, wherein the first wall forms a waveguide for propagating detectable signals therein; and
a second wall formed of a filtering material, for filtering excitation energy provided to the lumen to excite the sample.

177. The capillary of claim 176, wherein the second wall circumscribes the first wall.

178. The capillary of claim 176, wherein the second wall is formed of extra mural absorption (EMA) glass.

179. The capillary of claim 178, wherein the EMA glass is tuned to filter specific wavelengths of light.

180. A capillary array for screening a plurality of samples, comprising:
a plurality of capillaries, held together into the array, wherein each capillary includes a first wall defining a lumen for retaining the sample, and a second wall circumscribing the first wall, for filtering excitation energy provided to the lumen to excite the sample.

181. The array of claim 180, wherein the second wall of each capillary is formed of a filtering material.

182. The array of claim 181, wherein the filtering material is EMA glass.

183. The array of claim 182, wherein the EMA glass is tuned to filter specific wavelengths of light.

184. The array of claim 180, further comprising interstitial material between adjacent capillaries.

185. The array of claim 184, wherein the interstitial material is adapted to absorb light.

186. A method for incubating a bioactivity or biomolecule of interest, comprising:
introducing a first component into at least a portion of a capillary of a capillary array, wherein each capillary of the capillary array comprises at least one wall defining a lumen for retaining the first component;
introducing air into the capillary behind the first component; and
introducing a second component into the capillary, wherein the second component is separated from the first component by the air.

187. The method of claim 186, wherein either the first or second component includes at least one particle of interest.

188. The method of claim 187, wherein the other of the first and second component includes a developer for causing an activity of interest by the particle of interest.

189. The method of claim 187, wherein the particle of interest is a molecule.

190. The method of claim 186, further comprising disrupting the air to combine the first component with the second component.

191. The method of claim 186, wherein the first and second components are liquids.

192. A method of incubating a sample of interest, comprising:
introducing a first liquid labeled with a detectable particle into a capillary of a capillary array, wherein each capillary of the capillary array comprises at least one wall defining a lumen for retaining the liquid and the detectable particle;
submersing one end of the capillary into a fluid bath containing a second liquid; and
evaporating the first liquid from the opposite end of the capillary to draw the second liquid into the capillary tube.

193. The method of claim 192, wherein the second liquid contains a developer for causing an activity of interest by the detectable particle.

194. The method of claim 193, wherein the developer includes at least one nutrient.

195. The method of claim 194, wherein the nutrient includes oxygen.

196. A method of incubating a sample of interest, comprising:
introducing a first liquid labeled with a detectable particle into a capillary of a capillary array, wherein each capillary of the capillary array comprises at least one wall defining a lumen for retaining the first liquid and the detectable particle, and wherein the at least one wall is coated with a binding material for binding the detectable particle to the at least one wall;
removing the first liquid from the capillary tube, wherein the bound detectable particle is maintained within the capillary; and
introducing a second liquid into the capillary tube.

197. The method of claim 196, wherein the binding material includes DNA.

198. The method of claim 197, wherein the binding material includes an antibody.

199. A method of incubating a sample of interest, comprising:
introducing a liquid labeled with a detectable particle into a capillary of a capillary array, wherein each capillary of the capillary array comprises at least one wall defining a lumen for retaining the liquid and the detectable particle;
introducing paramagnetic beads to the liquid; and
exposing the capillary containing the paramagnetic beads to a magnetic field to cause movement of the paramagnetic beads in the liquid within the capillary.

200. The method of claim 199, further comprising reversing polarity of the magnetic field to cause reverse movement of the paramagnetic beads.

201. A method of recovering a sample from one of a plurality of capillaries in a capillary array, comprising:
determining a coordinate position of a recovery tool;
detecting a coordinate location of a capillary containing the sample;
correlating, via relative movement between the recovery tool and the capillary containing the sample, the coordinate position of the recovery tool with the coordinate location of the capillary; and
providing contact between the capillary and the recovery tool.

202. The method of claim 201, further comprising removing, with the recovery tool, the sample from the capillary containing the sample.

203. A recovery apparatus for a sample screening system, wherein the system includes a
plurality of capillaries formed into an array, the apparatus comprising:
a recovery tool adapted to contact at least one capillary of the capillary array and recover a sample therefrom;
an ejector, connected with the recovery tool, for ejecting the recovered sample from the recovery tool.

204. The recovery apparatus of claim 203, wherein the recovery tool includes a needle connected with a collection container.

205. The recovery apparatus of claim 203, wherein the recovery tool includes an aspirator for recovering the sample.

206. The recovery apparatus of claim 203, wherein the ejector includes a jet mechanism adapted to expel the recovered sample.

207. The recovery apparatus of claim 203, wherein the jet mechanism is operable by thermal energy applied thereto.

208. The recovery apparatus of claim 207, further comprising a heating element connected to the jet mechanism.

209. A sample screening apparatus, comprising:
a plurality of capillaries held together in a planar array, wherein each capillary comprises at least one wall defining a lumen for retaining a sample;
interstitial material disposed between adjacent capillaries in the array; and
one or more reference indicia formed within of the interstitial material.

210. The sample screening apparatus of claim 209, wherein the planar array includes approximately 1,000,000 capillaries.

211. A method of enriching for a polynucleotide encoding an activity of interest, comprising:
contacting a mixed population of polynucleotides derived from a mixed population of organisms with at least one nucleic acid probe comprising a detectable label and at least a portion of a polynucleotide sequence encoding a polypeptide of interest having a specified activity to enrich for polynucleotides positive for a sequence of interest.
